SEQUENCE LISTING (32 sequences)

SEQ ID NO: 1 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 223 aa, synthetic construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 413 aa, synthetic construct
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGSGGGSGGGGSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 418 aa, synthetic construct
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGSGGGSGGGGSGGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 417 aa, synthetic construct
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGSGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 419 aa, synthetic construct
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGSGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 412 aa, synthetic construct
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGSGGGSGGGGSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 11 - DNA, 1239 bp, synthetic construct
  source 1..1239: mol_type = other DNA
acccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggatccggtggcggttccggtggaggcggaagcggcggtggaggatcagtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 16 aa, synthetic construct
GSGGGSGGGGSGGGGS

SEQ ID NO: 13 - protein, 6 aa, synthetic construct
ASTKGP

SEQ ID NO: 14 - protein, 6 aa, synthetic construct
GGSGGS

SEQ ID NO: 15 - protein, 6 aa, synthetic construct
SGGGGS

SEQ ID NO: 16 - protein, 12 aa, synthetic construct
GRAGGGGAGGGG

SEQ ID NO: 17 - protein, 6 aa, synthetic construct
GRAGGG

SEQ ID NO: 18 - protein, 4 aa, synthetic construct
  VARIANT 2..4: note = can be absent
  VARIANT 1: note = can be present in repeats of any integer
GGGG

SEQ ID NO: 19 - protein, 4 aa, synthetic construct
  VARIANT 3..4: note = can be absent, or present in repeats of any integer
GSGS

SEQ ID NO: 20 - protein, 5 aa, synthetic construct
  VARIANT 1..5: note = can be present in repeats of any integer
GSGGS

SEQ ID NO: 21 - protein, 6 aa, synthetic construct
GPGPGP

SEQ ID NO: 22 - protein, 5 aa, synthetic construct
  VARIANT 1..5: note = can be present in repeats of any integer
GGGGS

SEQ ID NO: 23 - skipped

SEQ ID NO: 24 - protein, 4 aa, synthetic construct
GGSG

SEQ ID NO: 25 - protein, 5 aa, synthetic construct
GGSGG

SEQ ID NO: 26 - protein, 5 aa, synthetic construct
GSGSG

SEQ ID NO: 27 - protein, 5 aa, synthetic construct
GSGGG

SEQ ID NO: 28 - protein, 5 aa, synthetic construct
GGGSG

SEQ ID NO: 29 - protein, 5 aa, synthetic construct
GSSSG

SEQ ID NO: 30 - protein, 15 aa, synthetic construct
GGGGSGGGGSGGGGS

SEQ ID NO: 31 - protein, 5 aa, synthetic construct
GGGGS

SEQ ID NO: 32 - protein, 5 aa, synthetic construct
ERKCC

(19) United States
(12) Patent Application Publication
(10) Pub. No.: US 2026/0053742 A1
(43) Pub. Date: Feb. 26, 2026

(54) FORMULATIONS COMPRISING G-CSF AND USES THEREOF

(71) Applicant: EVIVE BIOTECHNOLOGY (SHANGHAI) LTD, Shanghai (CN)

(72) Inventors: Linfeng XU, Shanghai (CN); Ying TAN, Shanghai (CN); Tiansheng LI (DECEASED), Camarillo, CA (US)

(21) Appl. No.: 19/104,951

(22) PCT Filed: Aug. 18, 2023
(86) PCT No.: PCT/CN2023/113809

§ 371 Date: Feb. 19, 2025

(30) Foreign Application Priority Data
Aug. 19, 2022 (WO) PCT/CN2022/113560

(51) Int. Cl. A61K 9/08; A61K 9/00; A61K 38/19; A61K 47/12; A61K 47/18; A61K 47/26; A61K 47/65

(52) U.S. Cl. CPC A61K 9/08; A61K 9/0019; A61K 38/193; A61K 47/12; A61K 47/183; A61K 47/26; A61K 47/65

(57) ABSTRACT

Provided herein are methods of treating chemotherapy-induced neutropenia in a patient in need thereof by administering to the patient an effective amount of Eflapegrastim. Also provided herein are methods of treating radiation-induced neutropenia in a patient in need thereof by administering to the patient an effective amount of Eflapegrastim.

### CROSS REFERENCE TO RELATED APPLICATIONS

This patent application is a U.S. national stage application under 35 U.S.C. § 371 of International Application No. PCT/CN2023/113809, filed internationally on Aug. 18, 2023, which claims priority benefit of International Application No. PCT/CN2022/113560 filed on Aug. 19, 2022, the contents of each of which are incorporated herein by reference in their entireties.

### REFERENCE TO AN ELECTRONIC SEQUENCE LISTING

The contents of the electronic sequence listing (720622001900SEQLIST.xml; Size: 34,303 bytes; and Date of Creation: Dec. 19, 2024) is herein incorporated by reference in its entirety.

### FIELD

The present disclosure relates in some aspects to formulations (e.g., pharmaceutical compositions) of granulocyte colony stimulating factor (G-CSF) and uses thereof.

### BACKGROUND

The present embodiments relate generally to G-CSF formulations that are useful for injection and general administration.

G-CSF is a hematopoietic glycoprotein produced by stromal cells, macrophages, endothelial cells, fibroblasts, and monocytes. G-CSF binds to G-CSF receptor (G-CSFR) expressed on precursor cells in the bone marrow to promote the growth and differentiation of various types of blood cells, e.g., neutrophils, which play critical roles in the body's defense against bacterial infections. G-CSF can also activate mature neutrophils to participate in immune response, or synergize with other hematopoietic growth factors such as stem cell factor, Flt-3 ligand, and GM-CSF to perform hematopoietic functions.

G-CSF formulations may be administrated to patients via subcutaneous injection. Both liquid and lyophilized dosage forms can be used for drug products. However, high-concentration G-CSF protein formulations may present many challenges in formulation development, especially for liquid formulation. At high protein concentrations, the stability of proteins can become problematic due to increased protein-protein interactions, leading to increased formation of soluble and insoluble protein aggregates. If protein concentration is near its apparent solubility limit, phase separation can occur through precipitation, gelation, and/or crystallization. Further, high protein concentrations, which are desirable for formulations intended for intramuscular or subcutaneous administration and to provide convenience to healthcare providers and patients, may need proportionally high concentrations of stabilizers, to achieve long-term protein stability. The resulting formulation may be hypertonic, which may cause injection pain due to tissue damage. Therefore, it is critical to balance the amount of stabilizers for stability and osmolality of concentrated protein formulations.

For these reasons, there is a need for development of G-CSF formulations that exhibit high protein concentrations without significantly increased protein aggregation, osmolality, and/or decreased protein stability.

### BRIEF SUMMARY

In some aspects, disclosed herein are compositions of G-CSF. In some embodiments, there is provided a pharmaceutical composition comprising (or consisting essentially of, or consisting of): (a) about 1 mg/mL to about 100 mg/mL G-CSF dimer; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant.

In some embodiments according to any of the pharmaceutical compositions describe above, the G-CSF dimer comprises two monomeric subunits, wherein each monomeric subunit comprises a G-CSF monomer and a dimerization domain. In some embodiments, the G-CSF monomer comprises the sequence of SEQ ID NO: 1. In some embodiments, the G-CSF monomer is connected to the dimerization domain via an optional linker (e.g., within each monomeric subunit). In some embodiments, the linker is about 6 to about 30 amino acids in length. In some embodiments, the linker comprises the sequence of SEQ ID NO: 12. In some embodiments, the dimerization domain comprises at least two cysteines capable of forming intermolecular disulfide bonds. In some embodiments, the dimerization domain comprises at least a portion of an Fc fragment. In some embodiments, the Fc fragment comprises CH2 and CH3 domains. In some embodiments, the Fc fragment is derived from IgG1 Fc, IgG2 Fc, IgG4 Fc, or a fragment or a variant thereof. In some embodiments, the Fc fragment comprises an amino acid sequence selected from the group consisting of SEQ ID NOs: 2-5. In some embodiments, the G-CSF monomer is N-terminal to the dimerization domain within each monomeric subunit. In some embodiments, each monomeric subunit comprises an amino acid sequence of any of SEQ ID NOs: 6-10, or a variant thereof having at least about 90% (e.g., at least about 95%) sequence identity to the amino acid sequence of any of SEQ ID NOs: 6-10. In some embodiments, each monomeric subunit comprises an amino acid sequence selected from the group consisting of SEQ ID NOs: 6-10, such as SEQ ID NO: 6.

In some embodiments according to any of the pharmaceutical compositions describe above, the G-CSF dimer is efbemalenograstim alfa.

In some embodiments according to any of the pharmaceutical compositions described above, the concentration of the G-CSF dimer is about 5 mg/mL to about 50 mg/mL, such as about 20 mg/mL.

In some embodiments according to any of the pharmaceutical compositions described above, the buffering agent is sodium acetate. In some embodiments, the concentration of the sodium acetate is about 1 mM to about 30 mM, such as about 10 mM.

In some embodiments according to any of the pharmaceutical compositions described above, the stabilizing agent is EDTA. In some embodiments, the concentration of the EDTA is about 0.1 mM to about 10 mM, such as about 1 mM.

In some embodiments according to any of the pharmaceutical compositions described above, the tonicity agent is sorbitol or sucrose. In some embodiments, the tonicity agent is sorbitol. In some embodiments, the pharmaceutical composition comprises about 1% (w/v) to about 8% (w/v) sorbitol, such as about 5% (w/v) sorbitol. In some embodiments, the tonicity agent is sucrose. In some embodiments, pharmaceutical composition comprises about 5% (w/v) to about 15% (w/v) sucrose, such as about 9% (w/v) sucrose.

In some embodiments according to any of the pharmaceutical compositions described above, the surfactant is selected from the group consisting of a polysorbate, a poloxamer, a polyoxyethelene alkyl ether, an alkyl phenyl polyoxyethylene ether, and a combination thereof. In some embodiments, the surfactant is polysorbate 20 (PS20) or polysorbate 80 (PS80). In some embodiments, the pharmaceutical composition comprises about 0.005% (w/v) to about 0.05% (w/v) PS20, such as about 0.01% (w/v) PS20.

In some embodiments according to any of the pharmaceutical compositions described above, the pH of the pharmaceutical composition is about 4.2 to about 6.2, such as about 4.8 to about 5.8, about 5.0 to about 5.4, or about 5.2.

In some embodiments according to any of the pharmaceutical compositions described above, the pharmaceutical composition comprises (or consists essentially of, or consists of): (a) about 20 mg/mL of the G-CSF dimer; (b) about 10 mM sodium acetate; (c) about 1 mM EDTA; (d) about 5% (w/v) sorbitol; and (e) about 0.01% (w/v) PS20, wherein the pharmaceutical composition has a pH of about 5.2.

In some embodiments according to any of the pharmaceutical compositions described above, the pharmaceutical composition is stable at 25±2° C. for at least about 1 month, such as stable at 25±2° C. for at least about 2 months. In some embodiments, the pharmaceutical composition is stable at 25±2° C. up to about 3 months.

In some embodiments according to any of the pharmaceutical compositions described above, the pharmaceutical composition has a purity of G-CSF dimer of at least about 90% as assessed by size exclusion-high performance liquid chromatography (SE-HPLC).

In some embodiments according to any of the pharmaceutical compositions described above, the pharmaceutical composition has an osmolality of about 268 mOsm/kg to about 360 mOsm/kg.

In some embodiments according to any of the pharmaceutical compositions described above, the pharmaceutical composition comprises main peak at least about 85% in relation to the total area of all peaks when measured by SE-HPLC after at least about 1 month (e.g., at least about 2 months) of storage under 25±2° C. In some embodiments, the pharmaceutical composition comprises main peak at least about 85% in relation to the total area of all peaks when measured by SE-HPLC after up to about 3 months of storage under 25±2° C.

In some embodiments according to any of the pharmaceutical compositions described above, the pharmaceutical composition comprises dimer less than about 2.5% in relation to the total area of all peaks when measured by SE-HPLC after at least about 1 month (e.g., at least about 2 months) of storage under 25±2° C. In some embodiments, the pharmaceutical composition comprises dimer less than about 2.5% in relation to the total area of all peaks when measured by SE-HPLC after up to about 3 months of storage under 25±2° C.

In some embodiments according to any of the pharmaceutical compositions described above, the pharmaceutical composition comprises an aggregate in no more than about 2% in relation to the total area of all peaks when measured by SE-HPLC after at least about 1 month (e.g., at least about 2 months) of storage under 25±2° C. In some embodiments, the pharmaceutical composition comprises an aggregate in no more than about 2% in relation to the total area of all peaks when measured by SE-HPLC after up to about 3 months of storage under 25±2° C.

In some embodiments according to any of the pharmaceutical compositions described above, the pharmaceutical composition comprises main peak at least about 85% in relation to the total area of all peaks when measured by SE-HPLC after at least about 36 months of storage under about 2° C. to about 8° C. In some embodiments, the pharmaceutical composition comprises main peak at least about 85% in relation to the total area of all peaks when measured by SE-HPLC after up to about 36 months of storage under about 2° C. to about 8° C.

In some embodiments according to any of the pharmaceutical compositions described above, the pharmaceutical composition comprises at least about 96% of the G-CSF dimer as measured by reduced capillary electrophoresis-sodium dodecyl sulfate (rCE-SDS) after at least about 1 month (e.g., at least about 2 months) of storage under 25±2° C. In some embodiments, the pharmaceutical composition comprises at least about 96% of the G-CSF dimer as measured by rCE-SDS after up to about 3 months of storage under 25±2° C.

In some embodiments according to any of the pharmaceutical compositions described above, the pharmaceutical composition comprises at least about 95.5% of the G-CSF dimer as measured by rCE-SDS after at least about 36 months of storage under about 2° C. to about 8° C. In some embodiments, the pharmaceutical composition comprises at least about 95.5% of the G-CSF dimer as measured by rCE-SDS after up to about 36 months of storage under about 2° C. to about 8° C.

In some embodiments according to any of the pharmaceutical compositions described above, the pharmaceutical composition comprises no more than about 1.5% high molecular weight species (HMWS) as measured by rCE-SDS after at least about 1 month (e.g., at least about 2 months) of storage under 25±2° C. In some embodiments, the pharmaceutical composition comprises no more than about 1.5% HMWS as measured by rCE-SDS after up to about 3 months of storage under 25±2° C.

In some embodiments according to any of the pharmaceutical compositions described above, the pharmaceutical composition comprises no more than about 1.5% high molecular weight species as measured by rCE-SDS after at least about 36 months of storage under about 2° C. to about 8° C. In some embodiments, the pharmaceutical composition comprises no more than about 1.5% HMWS as measured by rCE-SDS after up to about 36 months of storage under about 2° C. to about 8° C.

In some embodiments according to any of the pharmaceutical compositions described above, the pharmaceutical composition comprises at least about 94% of the G-CSF dimer as measured by non-reduced capillary electrophoresis-sodium dodecyl sulfate (nrCE-SDS) after at least about 1 month (e.g., at least about 2 months) of storage under 25±2° C. In some embodiments, the pharmaceutical composition comprises at least about 94% of the G-CSF dimer as measured by nrCE-SDS after up to about 3 months of storage under 25±2° C.

In some embodiments according to any of the pharmaceutical compositions described above, the pharmaceutical composition comprises at least about 94% of G-CSF dimer as measured by nrCE-SDS after at least about 36 months of storage under about 2° C. to about 8° C. In some embodiments, the pharmaceutical composition comprises at least about 94% of G-CSF dimer as measured by nrCE-SDS after up to about 36 months of storage under about 2° C. to about 8° C.

In some embodiments according to any of the pharmaceutical compositions described above, the pharmaceutical composition comprises no more than about 1.5% high molecular weight species as measured by nrCE-SDS after at least about 1 month (e.g., at least about 2 months) of storage under 25±2° C. In some embodiments, the pharmaceutical composition comprises no more than about 1.5% HMWS as measured by nrCE-SDS after up to about 3 months of storage under 25±2° C.

In some embodiments according to any of the pharmaceutical compositions described above, the pharmaceutical composition comprises no more than about 1.5% high molecular weight species as measured by nrCE-SDS after at least about 36 months of storage under about 2° C. to about 8° C. In some embodiments, the pharmaceutical composition comprises no more than about 1.5% high molecular weight species as measured by nrCE-SDS after up to about 36 months of storage under about 2° C. to about 8° C.

In some embodiments according to any of the pharmaceutical compositions described above, the pharmaceutical composition comprises about 27.2% to about 39.0% acidic region, about 36.5% to about 60.1% main peak, and about 7.2% to about 30% basic region as measured by imaged capillary isoelectric focusing (icIEF) after at least about 1 month (e.g., at least about 2 months) of storage under 25±2° C. In some embodiments, the pharmaceutical composition comprises about 27.2% to about 39.0% acidic region, about 36.5% to about 60.1% main peak, and about 7.2% to about 30% basic region as measured by icIEF after up to about 3 months of storage under 25±2° C.

In some embodiments according to any of the pharmaceutical compositions described above, the pharmaceutical composition comprises about 27.2% to about 39.0% acidic region, about 36.5% to about 60.1% main peak, and about 7.2% to about 30% basic region as measured by icIEF after at least about 36 months of storage under about 2° C. to about 8° C. In some embodiments, the pharmaceutical composition comprises about 27.2% to about 39.0% acidic region, about 36.5% to about 60.1% main peak, and about 7.2% to about 30% basic region as measured by icIEF after up to about 36 months of storage under about 2° C. to about 8° C.

In some embodiments according to any of the pharmaceutical compositions described above, the pharmaceutical composition retains at least about 70% potency after at least about 1 month (e.g., at least about 2 months) of storage under 25±2° C. In some embodiments, the pharmaceutical composition retains at least about 70% potency after up to about 3 months of storage under 25±2° C.

In some embodiments according to any of the pharmaceutical compositions described above, the pharmaceutical composition retains at least about 70% potency after at least about 36 months of storage under about 2° C. to about 8° C. In some embodiments, the pharmaceutical composition retains at least about 70% potency after up to about 36 months of storage under about 2° C. to about 8° C.

In some embodiments according to any of the pharmaceutical compositions described above, the concentration of the pharmaceutical composition varies by less than about 0.5 mg/mL after at least about 1 month (e.g., at least about 2 months) of storage under 25±2° C. In some embodiments, the concentration of the pharmaceutical composition varies by less than about 0.5 mg/mL after up to about 3 months of storage under 25±2° C.

In some embodiments according to any of the pharmaceutical compositions described above, the concentration of pharmaceutical composition varies by less than about 0.5 mg/mL after at least about 36 months of storage under about 2° C. to about 8° C. In some embodiments, the concentration of pharmaceutical composition varies by less than about 0.5 mg/mL after up to about 36 months of storage under about 2° C. to about 8° C.

In some embodiments according to any of the pharmaceutical compositions described above, the pharmaceutical composition is contained in a syringe. In some embodiments, the volume of the pharmaceutical composition within the syringe is about 1 mL. In some embodiments, the syringe is for single use. In some embodiments, the syringe is sterile.

In another aspect, there is provided a syringe comprising any of the pharmaceutical compositions described above. In some embodiments, the syringe is for single use. In some embodiments, the volume of the liquid pharmaceutical composition within the syringe is about 1 mL. In some embodiments, the syringe is sterile.

In a further aspect, there is provided a method of treating or preventing a disease or condition in an individual (e.g., human), comprising administering to the individual an effective amount of any of the pharmaceutical compositions (e.g., liquid pharmaceutical composition) provided herein. In some embodiments, the pharmaceutical composition is administered at a dosage of from about 0.01 mg/kg to about 1 mg/kg. In some embodiments, the pharmaceutical composition is administered once every four weeks. In some embodiments, the pharmaceutical composition is administered once every three weeks. In some embodiments, the pharmaceutical composition is administered subcutaneously. In some embodiments, the disease or condition is selected from the group consisting of neutropenia, stroke, spinal injury, neurological disorders accompanied with blood brain barrier injury, Parkinson's disease, Alzheimer's disease, Huntington disease, amyotrophic lateral sclerosis, spinal muscular atrophy, primary lateral sclerosis, spinal cerebellar ataxias, and mobilization of hematopoietic stem cells into peripheral blood in allogeneic blood stem cell transplantation. In some embodiments, the disease or condition is neutropenia. In some embodiments, the neutropenia is chemotherapy-induced neutropenia or radiotherapy-induced neutropenia. In some embodiments, the method comprises administering to the individual the effective amount of the pharmaceutical composition after administration of a chemotherapeutic agent to the individual. In some embodiments, the chemotherapeutic agent is a myelosuppressive chemotherapeutic agent. In some embodiments, the pharmaceutical composition is administered at about 20 mg once per chemotherapy cycle. In some embodiments, the individual is administered at least 4 cycles of the chemotherapeutic agent, and wherein the pharmaceutical composition is administered after the administration of the chemotherapeutic agent in each cycle of the at least 4 cycles. In some embodiments, the pharmaceutical composition is administered at about 5 mg to about 25 mg (e.g., about 20 mg) of the G-CSF dimer for each administration.

In a further aspect, there is provided a method of manufacturing any of the pharmaceutical compositions disclosed herein, comprising: (i) culturing a host cell under a condition suitable for expressing the G-CSF dimer; (ii) isolating the expressed G-CSF dimer from the cell culture; (iii) purifying the expressed G-CSF dimer; and (iv) formulating the purified G-CSF dimer with the buffering agent, the stabilizing agent, the tonicity agent, and the surfactant. In some embodiments, the purification step comprises one or more of: affinity chromatography, viral inactivation, ion exchange chromatography, mixed-mode chromatography, and filtration.

### BRIEF DESCRIPTION OF THE DRAWINGS

FIG. 1 is an illustration of the structure of an exemplary G-CSF dimer (e.g., efbemalenograstim alfa). In the figure, “-” represents the linker, the oval-shaped object labeled with “G-CSF” represents a G-CSF monomer, and the oval-shaped object labeled with “Fc” represents an Fc fragment (e.g., human IgG-derived Fc). The exemplary G-CSF dimer comprises 2 monomeric subunits. Each monomeric subunit from N′ to C′ comprises a G-CSF monomer, a linker, and an Fc fragment.

FIG. 2 is an exemplary chromatogram by reverse phase high-performance liquid chromatography (RP-HPLC) of a G-CSF-Fc dimer pharmaceutical composition containing 15 mg/mL G-CSF-Fc dimer, 10 mM sodium acetate, 5% (w/v) sorbitol, 0.01% (w/v) polysorbate 20, 1 mM EDTA, pH 5.2, after stored for 12 weeks at 25±2° C. There are primarily three elution peaks, namely Impurity peak 1, Impurity peak 2, and Main peak, respectively.

FIGS. 3A-3B show the change in % main band of G-CSF dimer as determined by non-reducing SDS-PAGE (FIG. 3A) and reducing SDS-PAGE (FIG. 3B) in G-CSF dimer pharmaceutical compositions comprising: (●) 10 mM sodium acetate, 5% (w/v) sorbitol, 1 mM EDTA, 0.01% (w/v) polysorbate 20, pH 4.8 (▪) 10 mM sodium acetate, 5% (w/v) sorbitol, 1 mM EDTA, 0.01% (w/v) polysorbate 20, pH 5.2, (▴) 10 mM sodium acetate, 9% (w/v) sucrose, 1 mM EDTA, 0.01% (w/v) polysorbate 20, pH 4.8, or (x) 10 mM sodium acetate, 9% (w/v) sucrose, 1 mM EDTA, 0.01% (w/v) polysorbate 20, pH 5.2, after storage at 25±2° C. for up to 12 weeks.

FIGS. 3C-3D show the % main peak versus time as measured by RP-HPLC (FIG. 3C) and the % impurity peak 2 versus time as measured by RP-HPLC (FIG. 3D) in G-CSF dimer pharmaceutical compositions comprising: (●) 10 mM sodium acetate, 5% (w/v) sorbitol, 1 mM EDTA, 0.01% (w/v) polysorbate 20, pH 4.8 (▪) 10 mM sodium acetate, 5% (w/v) sorbitol, 1 mM EDTA, 0.01% (w/v) polysorbate 20, pH 5.2, (▴) 10 mM sodium acetate, 9% (w/v) sucrose, 1 mM EDTA, 0.01% (w/v) polysorbate 20, pH 4.8, or (x) 10 mM sodium acetate, 9% (w/v) sucrose, 1 mM EDTA, 0.01% (w/v) polysorbate 20, pH 5.2, after storage at 25±2° C. for up to 12 weeks.

FIGS. 4A-4B show the change in % main band of G-CSF dimer as determined by non-reducing SDS-PAGE (FIG. 4A) and reducing SDS-PAGE (FIG. 4B) in G-CSF dimer pharmaceutical compositions comprising: (●) 10 mM sodium acetate, 5% (w/v) sorbitol, 0.5 mM EDTA, 0.01% (w/v) polysorbate 20, pH 5.2 (▪) 10 mM sodium acetate, 5% (w/v) sorbitol, 1 mM EDTA, 0.01% (w/v) polysorbate 20, pH 5.2, or (▴) 10 mM sodium acetate, 5% (w/v) sorbitol, 0.5 mM EDTA, 0.03% (w/v) polysorbate 20, pH 5.2, after storage at 25±2° C. for up to 12 weeks.

FIGS. 4C-4D show the % main peak versus time as measured by RP-HPLC (FIG. 4C) and the % impurity peak 2 versus time as measured by RP-HPLC (FIG. 4D) in G-CSF dimer pharmaceutical compositions comprising: (●) 10 mM sodium acetate, 5% (w/v) sorbitol, 0.5 mM EDTA, 0.01% (w/v) polysorbate 20, pH 5.2 (▪) 10 mM sodium acetate, 5% (w/v) sorbitol, 1 mM EDTA, 0.01% (w/v) polysorbate 20, pH 5.2, or (▴) 10 mM sodium acetate, 5% (w/v) sorbitol, 0.5 mM EDTA, 0.03% (w/v) polysorbate 20, pH 5.2, after storage at 25±2° C. for up to 12 weeks.

FIGS. 4E-4F show the change in % main band of G-CSF dimer as determined by non-reducing SDS-PAGE (FIG. 4E) and reducing SDS-PAGE (FIG. 4F) in G-CSF dimer pharmaceutical compositions comprising: (x) 10 mM sodium acetate, 9% (w/v) sucrose, 0.5 mM EDTA, 0.01% (w/v) polysorbate 20, pH 5.2 (♦) 10 mM sodium acetate, 9% (w/v) sucrose, 1 mM EDTA, 0.01% (w/v) polysorbate 20, pH 5.2, or (∘) 10 mM sodium acetate, 9% (w/v) sucrose, 0.5 mM EDTA, 0.03% (w/v) polysorbate 20, pH 5.2, after storage at 25±2° C. for up to 12 weeks.

FIGS. 4G-4H shows the % main peak versus time as measured by RP-HPLC (FIG. 4G) and the % impurity peak 2 versus time as measured by RP-HPLC (FIG. 4H) in G-CSF dimer pharmaceutical compositions comprising: (x) 10 mM sodium acetate, 9% (w/v) sucrose, 0.5 mM EDTA, 0.01% (w/v) polysorbate 20, pH 5.2 (♦) 10 mM sodium acetate, 9% (w/v) sucrose, 1 mM EDTA, 0.01% (w/v) polysorbate 20, pH 5.2, or (∘) 10 mM sodium acetate, 9% (w/v) sucrose, 0.5 mM EDTA, 0.03% (w/v) polysorbate 20, pH 5.2, after storage at 25±2° C. for up to 12 weeks.

FIG. 5 is a chromatogram by size exclusion high-performance liquid chromatography (SE-HPLC) of a G-CSF-Fc dimer pharmaceutical composition containing 20 mg/mL G-CSF-Fc dimer, 10 mM sodium acetate, 5% (w/v) sorbitol, 0.01% (w/v) polysorbate 20, 1 mM EDTA, pH 5.2, at week 0. There are primarily two elution peaks, namely Dimer and Main Peak, respectively.

FIG. 6 shows the % Dimer versus time as measured by SE-HPLC in G-CSF dimer pharmaceutical compositions comprising: (●) 10 mM sodium acetate, 5% (w/v) sorbitol, 0.5 mM EDTA, 0.01% (w/v) polysorbate 20, pH 5.2 (▪) 10 mM sodium acetate, 5% (w/v) sorbitol, 1 mM EDTA, 0.01% (w/v) polysorbate 20, pH 5.2, or (▴) 10 mM sodium acetate, 5% (w/v) sorbitol, 0.5 mM EDTA, 0.03% (w/v) polysorbate 20, pH 5.2, after storage at 25±2° C. for up to 12 weeks.

### DETAILED DESCRIPTION

The present application provides G-CSF formulations (e.g., pharmaceutical composition) in liquid or lyophilized forms that are stable under suitable storage conditions. The application is based on the surprising finding that formulations comprising suitable excipients and G-CSF molecules, particularly a G-CSF dimer (e.g., G-CSF-Fc dimer, such as efbemalenograstim alfa), are highly stable upon storage. In some embodiments, the G-CSF formulation comprises at least about 97% (such as at least about any of 98%, 99%, or more) of G-CSF dimer as measured by size exclusion chromatography (SEC) before and/or after storage. In some embodiments, the G-CSF formulation comprises at least about 95.5% of G-CSF dimer as measured by rCE-SDS before and/or after storage (e.g., after at least about 1 month of storage under 25±2° C., or after at least about 36 months of storage under about 2° C. to about 8° C.). In some embodiments, the G-CSF formulation comprises at least about 94% (e.g., at least about 95%) of G-CSF dimer as measured by nrCE-SDS before and/or after storage (e.g., after at least about 1 month of storage under 25±2° C., or after at least about 36 months of storage under about 2° C. to about 8° C.). In some embodiments, the G-CSF formulation comprises no more than about 1.5% (e.g., no more than about 1%) of high molecular weight species (HMWS) as measured by rCE-SDS before and/or after storage (e.g., after at least about 1 month of storage under 25±2° C., or after at least about 36 months of storage under about 2° C. to about 8° C.). In some embodiments, the G-CSF formulation comprises no more than about 1.5% (e.g., no more than about 0.5%) of HMWS as measured by nrCE-SDS before and/or after storage (e.g., after at least about 1 month of storage under 25±2° C., or after at least about 36 months of storage under about 2° C. to about 8° C.). In some embodiments, the G-CSF formulation comprises at least about 50% of main peak as measured by icIEF before and/or after storage (e.g., after at least about 1 month of storage under 25±2° C., or after at least about 36 months of storage under about 2° C. to about 8° C.). In some embodiments, the G-CSF formulation retains at least about 70% (e.g., at least about any of 80%, 85%, 90%, 95%, 99%, or more) potency before and/or after storage (e.g., after at least about 1 month of storage under 25±2° C., or after at least about 36 months of storage under about 2° C. to about 8° C.). In some embodiments, the concentration of the G-CSF formulation varies by less than about 0.5 mg/mL before and/or after storage (e.g., after at least about 1 month of storage under 25±2° C., or after at least about 36 months of storage under about 2° C. to about 8° C.). These properties make the G-CSF formulation particularly suitable as a pharmaceutical product that can be safely administered to human patients, for example by subcutaneous administration. In some embodiments, the pharmaceutical compositions described herein provide high concentration of G-CSF (e.g., G-CSF dimer) in small volumes (e.g., 1 mL), which is for ease of therapeutic administration or delivery.

In addition, the G-CSF formulations (e.g., G-CSF-Fc dimer formulation) described herein maintain the osmolality of the formulation for improved in vivo tolerability and, consequently, increased patient compliance. In some embodiments, the osmolality of the G-CSF formulation is about 268 mOsm/kg to about 360 mOsm/kg, such as about 310 mOsm/kg. Moreover, the presently described formulations permit convenient processing, including but not limited to, ultrafiltration and sterile filtration, and injection of the drug product solution through the needle.

Thus, the present application in one aspect provides a pharmaceutical composition comprising (or consisting essentially of, or consisting of): (a) about 1 mg/mL to about 100 mg/mL G-CSF dimer (e.g., G-CSF-Fc dimer, such as efbemalenograstim alfa); (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, the pH of the pharmaceutical composition is about 4.2 to about 6.2 (e.g., about 5.0 to about 5.4, such as about 5.2). In another aspect, there is provided a method of assessing suitability of a pharmaceutical composition for medical use in an individual (e.g., human individual), wherein the pharmaceutical composition comprises (or consists essentially of, or consists of): (a) about 1 mg/mL to about 100 mg/mL G-CSF dimer (e.g., G-CSF-Fc dimer, such as efbemalenograstim alfa); (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant, wherein the pH of the pharmaceutical composition is about 4.2 to about 6.2 (e.g., about 5.0 to about 5.4, such as about 5.2), the method comprising measuring a quality control parameter for the pharmaceutical composition, and assessing the suitability of the pharmaceutical composition for medical use in an individual (e.g., human individual), wherein a measured quality control parameter within a quality control parameter threshold is indicative of suitability of the pharmaceutical composition for medical use. The quality control parameters can be any one or a combination of multiple parameters described herein for the pharmaceutical composition, for example, as assessed by corresponding methods described herein.

Also provided are syringes containing any of the pharmaceutical compositions described herein (e.g., efbemalenograstim alfa pharmaceutical composition), such as a syringe that is sterile and/or for single use.

Also provided are methods of treatment using the pharmaceutical compositions described herein as well as kits and articles of manufacturing useful for methods described herein.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

As used herein, the term “viscosity” refers to the resistance of a liquid formulation to flow, such as when injected through a syringe needle during administration to a patient. Viscosity measurements can be done by a cone and plate technique with a Peltier element set at a defined temperature, such as 5° C. to 25° C. as described herein. Typically, a well-defined shear stress gradient is applied to the liquid formulation and the resulting shear rate is measured. The viscosity is the ratio of the shear stress to the shear rate. As used herein, viscosity is expressed in units of mPa-S at 5° C. to 25° C. wherein 1 mPa-S=1 cP. The high concentration, low viscosity, substantially isosmotic formulations disclosed herein are typically characterized by having a viscosity ranging from 1 to 35 mPa-S at 5° C. to 25° C.

As used herein, the term “osmolality” refers to a measure of solute concentration, defined as the number of millimole of solute (both non-ionized and ionized forms) per kg of solution. A desired level of osmolality can be achieved by addition of one or more stabilizers such as a sugar or sugar alcohol including, but not limited to, mannitol, dextrose, glucose, trehalose, and/or sucrose. Additional stabilizers that are suitable for providing osmolality are described in references such as the handbook of Pharmaceutical Excipients (Fourth Edition, Royal Pharmaceutical Society of Great Britain, Science & Practice Publishers) or Remingtons: The Science and Practice of Pharmacy (Nineteenth Edition, Mack Publishing Company).

As used herein, the term “about” refers to +/−10% of the unit value provided. Reference to “about” a value or parameter herein includes (and describes) variations that are directed to that value or parameter per se. For example, description referring to “about X” includes description of “X”.

The term “about X-Y” used herein has the same meaning as “about X to about Y.”

As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result. The term “substantially” is therefore used herein to capture the potential lack of completeness inherent in many biological and chemical phenomena.

As used herein, the terms “isosmotic” and “isotonic” are used interchangeably with the terms “substantially isosmotic,” and “substantially isotonic” and refer to formulations characterized by having an osmotic pressure that is the same as or at least substantially equivalent to the osmotic pressure of another solution, which is achieved by formulations wherein the total concentration of solutes, including both permeable and impermeable solutes, in the formulation are the same as or at least substantially equivalent to the total number of solutes in another solution. Thus, while it will be appreciated by those of skill in the art that “isosmotic” and “isotonic” formulations that are used for in vivo administration generally have an osmolality ranging from about 268 mOsm/kg to about 360 mOsm/kg, in the context of the high concentration, low viscosity formulations of the present embodiments, the terms “isosmotic,” “isotonic,” “substantially isosmotic,” and “substantially isotonic” are used interchangeably to refer to formulations having an osmolality ranging from about 240 mOsm/kg to about 400 mOsm/kg, or from about 270 mOsm/kg to about 370 mOsm/kg, or from about 300 mOsm/kg to about 330 mOsm/kg.

As used herein, the term “tonicity agent” or “tonicity excipient” refers to compounds designed to reduce local irritation caused by pharmaceutical formulations/compositions by preventing osmotic shock at the site of application. Usually added to injectable, ocular or nasal preparations, these excipients include but are not limited to potassium chloride, sodium chloride, mannitol, sorbitol, dextrose, and glycerin.

As used herein, the term “buffering agent” refers to a substance that helps maintain the pH of an aqueous solution in the desired range. Suitable buffering agents include, for example, citric acid, sodium citrate, phosphoric acid, potassium phosphate, and various other acids and salts. In some aspects, a mixture of two or more buffering agents is used.

As used herein, the term “stabilizing agent” refers to a substance that helps maintain the desirable properties of the product. Exemplary stabilizing agents include, but are not limited to, amino acids, mannitol, sorbitol, sucrose, trehalose, dextran 40, and any combination thereof.

As used herein, the term “salt” refers to inorganic salts, which include but not limited to sodium chloride (NaCl), sodium sulfate (Na₂SO₄), sodium thiocyanate (NaSCN), magnesium chloride (MgCl₂), magnesium sulfate (MgSO₄), ammonium thiocyanate (NH₄SCN), ammonium sulfate ((NH₄)₂SO₄), ammonium chloride (NH₄Cl), calcium chloride (CaCl₂)), calcium sulfate (CaSO₄), zinc chloride (ZnCl₂) and the like, or combinations thereof.

As used herein, the term “surfactant” includes non-ionic surfactants including, without limitation, polysorbates, such as polysorbate 20 or 80, and polyoxamers, such as poloxamer 184 or 188. Amounts of surfactants effective to provide stable high concentration G-CSF dimer and other protein formulations are usually in the range of 0 ppm to 2000 ppm. The use of non-ionic surfactants permits the formulations to be exposed to shear and surface stresses without causing denaturation of the G-CSF dimer or other protein, and also reduce the adsorption on the surfaces during processing and storage. The formulations disclosed herein include, without limitation, formulations having one or more non-ionic surfactant (s) including, for example, one or more polysorbate (s), such as polysorbate 20 or 80; one or more polyoxamers, such as poloxamer 184 or 188. Exemplified herein are formulations having a polysorbate, such as polysorbate 20 (Tween® 20) or polysorbate 80 (Tween® 80).

As used herein, the term “protein” refers to amino acid polymers that contain at least five constituent amino acids that are covalently joined by peptide bonds. The constituent amino acids can be from the group of amino acids that are encoded by the genetic code, which include: alanine, valine, leucine, isoleucine, methionine, phenylalanine, tyrosine, tryptophan, serine, threonine, asparagine, glutamine, cysteine, glycine, proline, arginine, histidine, lysine, aspartic acid, and glutamic acid.

As used herein, the term “protein” is synonymous with the related terms “peptide” and “polypeptide”.

As used herein, the term “antibody” includes a full-length antibody, a bispecific antibody, a single-chain Fv (scFv) fragment, a Fab fragment, a Fab′ fragment, a F (ab′)₂, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody. “Antibody” may refer to a class of proteins that are generally known as immunoglobulins. Antibodies include full-length monoclonal antibodies (mAb), such as IgG4 monoclonal antibodies, which include immunoglobulin Fc regions.

As used herein, the term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H}) and a heavy chain constant region (abbreviated herein as C_{H}). The heavy chain constant region typically is comprised of three domains, C_{H}1, C_{H}2, and C_{H}3. Each light chain typically is comprised of a light chain variable region (abbreviated herein as V_{L}) and a light chain constant region. The light chain constant region typically is comprised of one domain, abbreviated herein as C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each V_{H} and V_{L} is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)

Unless otherwise specified herein, numbering of amino acid residues in the IgG Fc region is according to the EU numbering system for antibodies, also called the EU index, as described in Kabat et al., Sequences of Proteins of Immunological Interest, 5th Ed. Public Health Service, National Institutes of Health, Bethesda, MD, 1991.

The “CH1 domain” (also referred to as “C1” of “H1” domain) usually extends from about amino acid 118 to about amino acid 215 (EU numbering system). “Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions. The “CH2 domain” of a human IgG Fc domain (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985). The “CH3 domain” (also referred to as “C3” domain) comprises the stretch of residues C-terminal to a CH2 domain in an Fc domain (i.e., from about amino acid residue 341 to the C-terminal end of an antibody sequence, typically at amino acid residue 446 or 447 of an IgG).

The term “Fc domain” or “fragment crystallizable region” herein is used to define a C-terminal region of an immunoglobulin heavy chain, including native-sequence Fc domains and variant Fc domains. Although the boundaries of the Fc domain of an immunoglobulin heavy chain might vary, the human IgG heavy-chain Fc domain is usually defined to stretch from an amino acid residue at position Cys226, or from Pro230, to the carboxyl-terminus thereof. The C-terminal lysine (residue 447 according to the EU numbering system) of the Fc domain may be removed, for example, during production or purification of the Fc-containing protein, or by recombinantly engineering the nucleic acid encoding an Fc-containing protein. Accordingly, a composition of Fc-containing protein may comprise Fc-containing protein populations with all K447 residues removed, Fc-containing protein populations with no K447 residues removed, and Fc-containing protein populations having a mixture of Fc-containing proteins with and without the K447 residue. Suitable native-sequence Fc domains for use in the Fc-containing protein described herein can include human IgG1, IgG2 (IgG2A, IgG2B), IgG3 and IgG4.

“Percent (%) amino acid sequence identity” or “homology” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

As used herein, the term “pharmaceutically effective amount” of a G-CSF dimer molecule formulation refers to an amount of the formulation that provides therapeutic effect in an administration regimen.

The terms “effective amount” and “pharmaceutically effective amount” as used herein refer to a sufficient amount of an agent to provide the desired biological result. That result can be reduction (e.g., reducing at least about any of 10%, 20%, 30%, 40%, 50%, 60%, 70%, 80%, 90%, 95%, 99%, or 100%) and/or alleviation of the signs, symptoms, or causes of a disease or disorder, or any other desired alteration of a biological system. As will be appreciated by those of ordinary skill in this art, the effective amount of an agent may vary depending on such factors as the desired biological endpoint, the pharmacokinetics of the compound, the disease being treated, the mode of administration, and the age, weight, health, and condition of the subject.

As used herein, the terms “pharmaceutical formulation” or “pharmaceutical composition” are used interchangeably herein and refer to a preparation which is in such form as to permit the biological activity of an active ingredient contained therein to be effective, and which contains no additional components which are unacceptably toxic to a subject to which the formulation would be administered.

A “sterile” formulation is aseptic or essentially free from living microorganisms and their spores.

As used herein, the term “absolute neutrophil count (ANC)” is a measure of the number of neutrophil granulocytes present in the blood. Neutrophils are a type of white blood cell that plays a central role in the immune system's defense against infections, particularly bacterial infections. They are the most abundant type of white blood cell in most mammals and form an essential part of the innate immune system.

As used herein, “myelosuppression” refers to the suppression of one or more components of hematopoiesis, which manifests in aberrant levels of one or more of the cell types that are the products of this process. For a review of hematopoiesis, and characteristics of hematopoietic cells, see CLINICAL IMMUNOLOGY: PRINCIPLES AND Practice, Vol. 1, Ch. 2, pp. 15-24 (Lewis and Harriman, eds. Mosby—Year Book, Inc. 1996), which pages are hereby incorporated by reference. On a general level it refers to decreases in white blood cell (WBC) and/or platelet counts. It also refers, on a more specific level, to suppression of one or more of the following cells that result from hematopoiesis: B-cells, T-cells, natural killer cells, dendritic cells, macrophages, neutrophils, eosinophils, basophils, mast cells and platelets. On the other hand, therefore, “myelorecovery” is the opposite of myelosuppression. A “myelosuppressive agent” or “myelosuppressive therapy” is an agent or therapy that can induce myelosuppression.

As used herein, the term “neutropenia” is defined as a condition characterized by a low number of neutrophils. Normal is defined as having an absolute neutrophil count greater than or equal to 2.0×10⁹/L. Grade 1 is defined as neutropenia having an absolute neutrophil count between 1.5×10⁹/L and 1.999×10⁹/L. Grade 2 is defined as neutropenia having an absolute neutrophil count between 1.0×10⁹/L and 1.499×10⁹/L. Grade 3 is defined as neutropenia having an absolute neutrophil count between 0.5×10⁹/L and 0.999×10⁹/L. Grade 4 (Severe Neutropenia) is defined as neutropenia having an absolute neutrophil count less than 0.5×10⁹/L. The terms “severe neutropenia” and “Grade 4 neutropenia” may be used interchangeably.

The term “febrile neutropenia” (FN) is defined as a single oral temperature of ≥38.3° C. (101° F.) or a temperature of ≥38.0° C. (100.4° F.) sustained over a 1 hour period, coupled with neutropenia, which is typically classified as an ANC of <500 cells/mm³ or an ANC that is expected to decrease to <500 cells/mm³ during the next 48 hours. It is a serious complication often seen in people undergoing cancer treatment, particularly chemotherapy.

A “subject” or “individual” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the individual is a human. In certain embodiments, the individual is a non-human animal. The individual can be of any age and/or sex. In some embodiments, the individual has cancer.

The terms “disease,” “disorder,” and “condition” are used interchangeably herein.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease (e.g., fever, infection, reduced ANC), diminishing the extent of the disease (e.g., lowering the grade of neutropenia), reducing the duration of the disease, stabilizing the disease (e.g., preventing or delaying the worsening of the disease), preventing or delaying the recurrence of the disease, delaying or slowing the progression of the disease (e.g., progression to the next grade of neutropenia or febrile neutropenia), ameliorating the disease state, providing a remission (partial or total) of the disease, decreasing the dose of one or more other medications required to treat the disease, increasing or improving the quality of life, increasing weight gain, and/or prolonging survival. Also encompassed by “treatment” is a reduction of pathological consequence (e.g., reduction of ANC, infection, fever), including the severity and incidence of a pathological consequence. The methods of the application contemplate any one or more of these aspects of treatment. When the method is for treating cancer, the method in some embodiments may also prevent or delay the spread of cancer (e.g., metastasis), and/or reduce of pathological consequence (e.g., tumor volume).

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease.

“Preventing” as used herein, includes providing prophylaxis with respect to the occurrence or recurrence of a disease in an individual that may be predisposed to the disease but has not yet been diagnosed with the disease.

It is understood that embodiments of the invention described herein include “consisting of” and/or “consisting essentially of” embodiments.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “or,” and “the” include plural referents unless the context clearly dictates otherwise.

### II. Pharmaceutical Compositions

In some embodiments, there is provided a pharmaceutical composition comprising a G-CSF molecule. Any of the G-CSF molecules described herein can be used in the pharmaceutical composition, including but are not limited to G-CSF dimers (e.g., any of the G-CSF dimers described herein). In some embodiments, the pharmaceutical composition comprises (or consists essentially of, or consists of): (a) about 1 mg/mL to about 100 mg/mL of a G-CSF molecule (e.g., G-CSF dimer, such as G-CSF-Fc dimer); (b) about 1 mM to about 50 mM buffering agent (e.g., sodium acetate); (c) about 0.1 mM to about 20 mM stabilizing agent (e.g., EDTA); (d) about 1% (w/v) to about 15% (w/v) (e.g., about 1% (w/v) to about 10% (w/v)) tonicity agent (e.g., sorbitol or sucrose); and (e) about 0.001% (w/v) to about 0.1% (w/v) surfactant (e.g., polysorbate 20 or polysorbate 80). In some embodiments, the pH of the pharmaceutical composition is about 4.2 to about 6.2, such as about 5.0 to about 5.4, or about 5.2. In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa.

In some embodiments, disclosed herein are pharmaceutical compositions comprising a G-CSF dimer, such as any of the G-CSF dimers described herein. In some embodiments, the G-CSF dimer comprises 2 monomeric subunits, wherein each monomeric subunit comprises a G-CSF monomer and a dimerization domain. In some embodiments, the G-CSF dimer has a prolonged serum half-life. In some embodiments, each monomeric subunit comprises (or consists essentially of, or consists of) an amino acid sequence of SEQ ID NO: 6. In some embodiments, the G-CSF dimer is efbemalenograstim alfa.

In some embodiments, the pharmaceutical composition comprises from about 1 mM to about 50 mM buffering agent (e.g., sodium acetate). In some embodiments, the pharmaceutical composition comprises about 0.1 mM to about 20 mM stabilizing agent (e.g., EDTA). In some embodiments, the pharmaceutical composition comprises from about 1% (w/v) to about 15% (w/v), such as about 1% (w/v) to about 10% (w/v), of tonicity agent (e.g., sorbitol or sucrose). In some embodiments, the pharmaceutical composition comprises from about 0.001% (w/v) to about 0.1% (w/v) of surfactant (e.g., polysorbate 20). In some embodiments, the pH of the pharmaceutical composition is about 4.2 to about 6.2, such as about 4.8 to about 5.8, about 5.0 to about 5.4, or about 5.2.

Typically, G-CSF dimers in the pharmaceutical compositions remain soluble at concentrations of between about 1 mg/mL and about 100 mg/mL and remain stable under isosmotic storage conditions and exhibit improved stability as compared to currently available G-CSF formulations. In some embodiments, the pharmaceutical composition comprises no less than about any of 1 mg/mL, 2 mg/mL, 3 mg/mL, 4 mg/mL, 5 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 95 mg/mL of G-CSF dimer. In some embodiments, the pharmaceutical composition comprises about 1 mg/mL to about 100 mg/mL G-CSF dimer, such as any of about 1 mg/mL to about 90 mg/mL, about 1 mg/mL to about 80 mg/mL, about 1 mg/mL to about 70 mg/mL, about 1 mg/mL to about 60 mg/mL, about 1 mg/mL to about 50 mg/mL, about 2 mg/mL to about 50 mg/mL, about 5 mg/mL to about 50 mg/mL, about 5 mg/mL to about 40 mg/mL, about 5 mg/mL to about 30 mg/mL, about 50 mg/mL to about 100 mg/mL, about 10 mg/mL to about 90 mg/mL, about 10 mg/mL to about 80 mg/mL, about 10 mg/mL to about 70 mg/mL, about 10 mg/mL to about 60 mg/mL, about 10 mg/mL to about 50 mg/mL, about 10 mg/mL to about 40 mg/mL, or about 10 mg/mL to about 25 mg/mL of G-CSF dimer. In some embodiments, the pharmaceutical composition comprises about 18 mg/mL to about 22 mg/mL of G-CSF dimer, such as about 20 mg/mL of G-CSF dimer.

Buffering agent may include, but are not limited to, citrates, phosphates, acetates, succinates, tartrates, maleates, HEPES, Tris, Bicine, glycine, N-glycylglycine, carbonates, glycylglycine, lysine, arginine, histidine, and/or mixtures thereof. In some embodiments, the buffering agent is sodium acetate. In some embodiments, the pharmaceutical composition comprises about 1 mM to about 50 mM sodium acetate, such as any of about 1 mM to about 40 mM, about 1 mM to about 30 mM, about 5 mM to about 25 mM, about 5 mM to about 20 mM, or about 10 mM sodium acetate.

In some embodiments, in the pharmaceutical compositions described herein, sodium acetate is a buffer agent that can be used to maintain the pharmaceutical composition pH from about pH 4.2 to about pH 6.2, such as any of about pH 4.4 to about pH 6.0, about pH 4.6 to about pH 5.8, about pH 4.8 to about pH 5.8, about pH 4.8 to about pH 5.6, or about pH 5.0 to about pH 5.4. In some embodiments, the pH of the pharmaceutical composition is about pH 5.0 to about pH 5.4 (pH 5.2±0.2), such as about pH 5.2.

A stabilizing agent may be added to improve the stability and prolong the shelf life of the pharmaceutical compositions described herein. Examples of stabilizing agents include, but are not limited to, glycine, alanine, glutamate, methionine, arginine, benzoic acid, citric, glycolic, lactic, malic, maleic acid, polyol (such as sorbitol, mannitol, and trehalose), surfactant, ethylenediaminetetraacetic acid (EDTA), diethylenetriaminepentaacetic acid (DTPA), hydroxyethylenediaminetriacetic acid (HEDTA), ethylene glycol-bis-(2-aminoethyl)-N, N, N′, N′-tetraacetic acid (EGTA), nitrilotriacetic acid (NTA), metal ion stabilizing agent and citrates. In some embodiments, the stabilizing agent is EDTA. In some embodiments, the pharmaceutical composition comprises about 0.1 mM to about 20 mM EDTA, such as any of about 0.1 mM to about 15 mM, about 0.1 mM to about 10 mM, about 0.5 mM to about 5 mM, about 0.5 mM to about 2 mM, about 1 mM to about 2 mM, or about 1 mM EDTA. EDTA may function as a chelating agent, a preservative or stabilizer to prevent catalytic oxidative reaction in the pharmaceutical composition during processing and storage.

In some embodiments, the pharmaceutical composition comprises one or more tonicity agents. Tonicity agents reduce (e.g., reducing at least about any of 10%, 20%, 30%, 40%, 50%, 60%, 70%, 80%, 90%, 95%, or 100%) local irritation caused by pharmaceutical formulations/compositions by preventing osmotic shock at the site of application. Examples of tonicity agents include, but are not limited to, a sugar alcohol or polyol (such as mannitol or polyol), a non-ionic surfactant (such as polysorbate 20, polysorbate 40, polysorbate 60, polysorbate 80), and a sugar (such as sucrose, maltose, trehalose). In some embodiments, the tonicity agent is sorbitol. In some embodiments, the pharmaceutical composition comprises about 1% (w/v) to about 15% (w/v) sorbitol, such as any of about 1% (w/v) to about 10% (w/v), about 2% (w/v) to about 10% (w/v), about 3% (w/v) to about 10% (w/v), about 4% (w/v) to about 10% (w/v), about 5% (w/v) to about 10% (w/v), about 5% (w/v) to about 9% (w/v), about 5% (w/v) to about 8% (w/v), about 5% (w/v) to about 7% (w/v), about 5% (w/v) to about 6% (w/v), or about 5% (w/v) sorbitol. In some embodiments, the tonicity agent is sucrose. In some embodiments, the pharmaceutical composition comprises about 1% (w/v) to about 15% (w/v) sucrose, such as any of about 5% (w/v) to about 15% (w/v), about 5% (w/v) to about 10% (w/v), about 6% (w/v) to about 15% (w/v), about 7% (w/v) to about 15% (w/v), about 8% (w/v) to about 15% (w/v), about 9% (w/v) to about 15% (w/v), about 9% (w/v) to about 14% (w/v), about 9% (w/v) to about 13% (w/v), about 9% (w/v) to about 12% (w/v), about 9% (w/v) to about 11% (w/v), about 9% (w/v) to about 10% (w/v), or about 9% (w/v) sucrose.

Non-ionic surfactants, such as polysorbates, including polysorbate (Tween®) 20 and polysorbate (Tween®) 80; polyoxamers, including poloxamer 184 and 188; Pluronic® polyols; and other ethylene/polypropylene block polymers, stabilize the formulation during processing and storage by reducing interfacial interaction and prevent protein from adsorption. In some embodiments, the surfactant is selected from the group consisting of a polysorbate, a poloxamer, a polyoxyethelene alkyl ether, an alkyl phenyl polyoxyethylene ether, and a combination thereof. In some embodiments, the surfactant is polysorbate 20 (PS20) or polysorbate 80 (PS80). In some embodiments, the pharmaceutical composition comprises polysorbate 20 (PS20). In some embodiments, the pharmaceutical composition comprises about 0.001% (w/v) to about 0.1% (w/v) polysorbate 20, such as any of about 0.001% (w/v) to about 0.09% (w/v), about 0.001% (w/v) to about 0.08% (w/v), about 0.001% (w/v) to about 0.07% (w/v), about 0.001% (w/v) to about 0.06% (w/v), about 0.001% (w/v) to about 0.05% (w/v), about 0.001% (w/v) to about 0.04% (w/v), about 0.001% (w/v) to about 0.03% (w/v), about 0.001% (w/v) to about 0.02% (w/v), about 0.001% (w/v) to about 0.01% (w/v), about 0.005% (w/v) to about 0.05% (w/v), about 0.008% (w/v) to about 0.012% (w/v), or about 0.01% (w/v) polysorbate 20.

Exemplified herein are G-CSF dimer pharmaceutical compositions wherein the G-CSF dimers are soluble at high protein concentrations and stable during long periods of storage.

In some embodiments, there is provided a pharmaceutical composition comprising (or consisting essentially of, or consisting of): (a) about 1 mg/mL to about 100 mg/mL (e.g., about 5 mg/mL to about 50 mg/mL, such as about 20 mg/mL) of a G-CSF dimer; (b) about 1 mM to about 50 mM (e.g., about 1 mM to about 30 mM, such as about 10 mM) buffering agent (e.g., sodium acetate); (c) about 0.1 mM to about 20 mM (e.g., about 0.1 mM to about 10 mM, such as about 1 mM) stabilizing agent (e.g., EDTA); (d) about 1% (w/v) to about 15% (w/v) (e.g., about 1% (w/v) to about 10% (w/v), such as about 5% (w/v) or about 9% (w/v)) tonicity agent (e.g., sorbitol or sucrose); and (e) about 0.001% (w/v) to about 0.1% (w/v) (e.g., about 0.005% (w/v) to about 0.05% (w/v), such as about 0.01% (w/v)) surfactant (e.g., polysorbate 20 or polysorbate 80); wherein the pH of the pharmaceutical composition is about 4.2 to about 6.2 (e.g., about 5.0 to about 5.4, or about 5.2); wherein the G-CSF dimer comprises two monomeric subunits, and wherein each monomeric subunit comprises a G-CSF monomer and a dimerization domain. In some embodiments, there is provided a pharmaceutical composition comprising (or consisting essentially of, or consisting of): (a) about 1 mg/mL to about 100 mg/mL (e.g., about 5 mg/mL to about 50 mg/mL, such as about 20 mg/mL) of a G-CSF dimer; (b) about 1 mM to about 50 mM (e.g., about 1 mM to about 30 mM, such as about 10 mM) buffering agent (e.g., sodium acetate); (c) about 0.1 mM to about 20 mM (e.g., about 0.1 mM to about 10 mM, such as about 1 mM) stabilizing agent (e.g., EDTA); (d) about 1% (w/v) to about 15% (w/v) (e.g., about 1% (w/v) to about 10% (w/v), such as about 5% (w/v) or about 9% (w/v)) tonicity agent (e.g., sorbitol or sucrose); and (e) about 0.001% (w/v) to about 0.1% (w/v) (e.g., about 0.005% (w/v) to about 0.05% (w/v), such as about 0.01% (w/v)) surfactant (e.g., polysorbate 20 or polysorbate 80); wherein the pH of the pharmaceutical composition is about 4.2 to about 6.2 (e.g., about 5.0 to about 5.4, or about 5.2); wherein the G-CSF dimer comprises two monomeric subunits, and wherein each monomeric subunit comprises a G-CSF monomer, an Fc fragment, and an optional linker connecting the G-CSF monomer and the Fc fragment. In some embodiments, the G-CSF monomer comprises the sequence of SEQ ID NO: 1. In some embodiments, the G-CSF monomer is connected to the dimerization domain via an optional linker, such as a linker comprising the amino acid sequence of SEQ ID NO: 12. In some embodiments, the dimerization domain comprises at least a portion of an Fc fragment. In some embodiments, the Fc fragment comprises CH2 and CH3 domains. In some embodiments, the Fc fragment is derived from IgG1 Fc, IgG2 Fc, IgG4 Fc, or a fragment or a variant thereof. In some embodiments, the Fc fragment comprises an amino acid sequence selected from the group consisting of SEQ ID NOs: 2-5. In some embodiments, the G-CSF monomer is N-terminal to the dimerization domain within each monomeric subunit. In some embodiments, each monomeric subunit comprises (or consists essentially of, or consists of) an amino acid sequence of any of SEQ ID NOs: 6-10, or a variant thereof having at least about 90% sequence identity to the amino acid sequence of any of SEQ ID NOs: 6-10. In some embodiments, there is provided a pharmaceutical composition comprising (or consisting essentially of, or consisting of): (a) about 1 mg/mL to about 100 mg/mL (e.g., about 5 mg/mL to about 50 mg/mL, such as about 20 mg/mL) of a G-CSF dimer; (b) about 1 mM to about 50 mM (e.g., about 1 mM to about 30 mM, such as about 10 mM) buffering agent (e.g., sodium acetate); (c) about 0.1 mM to about 20 mM (e.g., about 0.1 mM to about 10 mM, such as about 1 mM) stabilizing agent (e.g., EDTA); (d) about 1% (w/v) to about 15% (w/v) (e.g., about 1% (w/v) to about 10% (w/v), such as about 5% (w/v) or about 9% (w/v)) tonicity agent (e.g., sorbitol or sucrose); and (e) about 0.001% (w/v) to about 0.1% (w/v) (e.g., about 0.005% (w/v) to about 0.05% (w/v), such as about 0.01% (w/v)) surfactant (e.g., polysorbate 20 or polysorbate 80); wherein the pH of the pharmaceutical composition is about 4.2 to about 6.2 (e.g., about 5.0 to about 5.4, or about 5.2); wherein the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, there is provided a pharmaceutical composition comprising (or consisting essentially of, or consisting of): (a) about 1 mg/mL to about 100 mg/mL (e.g., about 5 mg/mL to about 50 mg/mL, such as about 20 mg/mL) of a G-CSF dimer; (b) about 1 mM to about 50 mM (e.g., about 1 mM to about 30 mM, such as about 10 mM) sodium acetate; (c) about 0.1 mM to about 20 mM (e.g., about 0.1 mM to about 10 mM, such as about 1 mM) EDTA; (d) about 1% (w/v) to about 15% (w/v) (e.g., about 1% (w/v) to about 10% (w/v), such as about 5% (w/v)) sorbitol; and (e) about 0.001% (w/v) to about 0.1% (w/v) (e.g., about 0.005% (w/v) to about 0.05% (w/v), such as about 0.01% (w/v)) surfactant (e.g., polysorbate 20 or polysorbate 80); wherein the pH of the pharmaceutical composition is about 4.2 to about 6.2 (e.g., about 5.0 to about 5.4, or about 5.2); wherein the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, there is provided a pharmaceutical composition comprising (or consisting essentially of, or consisting of): (a) about 1 mg/mL to about 100 mg/mL (e.g., about 5 mg/mL to about 50 mg/mL, such as about 20 mg/mL) of a G-CSF dimer; (b) about 1 mM to about 50 mM (e.g., about 1 mM to about 30 mM, such as about 10 mM) sodium acetate; (c) about 0.1 mM to about 20 mM (e.g., about 0.1 mM to about 10 mM, such as about 1 mM) EDTA; (d) about 1% (w/v) to about 15% (w/v) (e.g., about 5% (w/v) to about 15% (w/v), such as about 9% (w/v)) sucrose; and (e) about 0.001% (w/v) to about 0.1% (w/v) (e.g., about 0.005% (w/v) to about 0.05% (w/v), such as about 0.01% (w/v)) surfactant (e.g., polysorbate 20 or polysorbate 80); wherein the pH of the pharmaceutical composition is about 4.2 to about 6.2 (e.g., about 5.0 to about 5.4, or about 5.2); wherein the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, there is provided a pharmaceutical composition comprising (or consisting essentially of, or consisting of): (a) about 20 mg/mL of a G-CSF dimer; (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.0 to about 5.4 (e.g., about 5.2); wherein the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile.

In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 50 mM (e.g., about 1 mM to about 30 mM, such as about 10 mM) buffering agent (e.g., sodium acetate); (c) about 0.1 mM to about 20 mM (e.g., about 0.1 mM to about 10 mM, such as about 1 mM) stabilizing agent (e.g., EDTA); (d) about 1% (w/v) to about 15% (w/v) (e.g., about 1% (w/v) to about 10% (w/v), such as about 5% (w/v) or about 9% (w/v)) tonicity agent (e.g., sorbitol or sucrose); and (e) about 0.001% (w/v) to about 0.1% (w/v) (e.g., about 0.005% (w/v) to about 0.05% (w/v), such as about 0.01% (w/v)) surfactant (e.g., polysorbate 20 or polysorbate 80); wherein the pH of the pharmaceutical composition is about 4.2 to about 6.2 (e.g., about 5.0 to about 5.4, or about 5.2). In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 50 mM (e.g., about 1 mM to about 30 mM, such as about 10 mM) sodium acetate; (c) about 0.1 mM to about 20 mM (e.g., about 0.1 mM to about 10 mM, such as about 1 mM) EDTA; (d) about 1% (w/v) to about 15% (w/v) (e.g., about 1% (w/v) to about 10% (w/v), such as about 5% (w/v)) sorbitol; and (e) about 0.001% (w/v) to about 0.1% (w/v) (e.g., about 0.005% (w/v) to about 0.05% (w/v), such as about 0.01% (w/v)) polysorbate 20 or polysorbate 80 (e.g., polysorbate 20); wherein the pH of the pharmaceutical composition is about 4.2 to about 6.2 (e.g., about 5.0 to about 5.4, or about 5.2). In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 50 mM (e.g., about 1 mM to about 30 mM, such as about 10 mM) sodium acetate; (c) about 0.1 mM to about 20 mM (e.g., about 0.1 mM to about 10 mM, such as about 1 mM) EDTA; (d) about 1% (w/v) to about 15% (w/v) (e.g., about 1% (w/v) to about 10% (w/v), such as about 9% (w/v)) sucrose; and (e) about 0.001% (w/v) to about 0.1% (w/v) (e.g., about 0.005% (w/v) to about 0.05% (w/v), such as about 0.01% (w/v)) polysorbate 20 or polysorbate 80 (e.g., polysorbate 20); wherein the pH of the pharmaceutical composition is about 4.2 to about 6.2 (e.g., about 5.0 to about 5.4, or about 5.2). In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile.

In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 50 mM sodium acetate; (c) about 0.1 mM to about 20 mM EDTA; (d) about 1% (w/v) to about 10% (w/v) sorbitol; and (e) about 0.001% (w/v) to about 0.1% (w/v) polysorbate 20. In some embodiments, there is provided a pharmaceutical composition comprising (or consisting essentially of, or consisting of): (a) about 1 mg/mL to about 100 mg/mL (e.g., about 20 mg/mL) of a G-CSF dimer (e.g., G-CSF-Fc dimer, such as efbemalenograstim alfa); (b) about 1 mM to about 50 mM sodium acetate; (c) about 0.1 mM to about 20 mM EDTA; (d) about 1% (w/v) to about 10% (w/v) sorbitol; and (e) about 0.001% (w/v) to about 0.1% (w/v) polysorbate 20. In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 50 mM sodium acetate; (c) about 0.1 mM to about 20 mM EDTA; (d) about 1% (w/v) to about 15% (w/v) sucrose; and (e) about 0.001% (w/v) to about 0.1% (w/v) polysorbate 20. In some embodiments, there is provided a pharmaceutical composition comprising (or consisting essentially of, or consisting of): (a) about 1 mg/mL to about 100 mg/mL (e.g., about 20 mg/mL) of a G-CSF dimer (e.g., G-CSF-Fc dimer, such as efbemalenograstim alfa); (b) about 1 mM to about 50 mM sodium acetate; (c) about 0.1 mM to about 20 mM EDTA; (d) about 1% (w/v) to about 15% (w/v) sucrose; and (e) about 0.001% (w/v) to about 0.1% (w/v) polysorbate 20. In some embodiments, the pH of the pharmaceutical composition is about 4.2 to about 6.2, such as about 5.0 to about 5.4, or about 5.2. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile.

In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 50 mM sodium acetate; (c) about 0.1 mM to about 20 mM EDTA; (d) about 1% (w/v) to about 10% (w/v) sorbitol; and (e) about 0.001% (w/v) to about 0.1% (w/v) polysorbate 20, wherein the pharmaceutical composition has a pH of about 4.2 to about 6.2 (such as about 5.0 to about 5.4, or about 5.2). In some embodiments, there is provided a pharmaceutical composition comprising (or consisting essentially of, or consisting of): (a) about 1 mg/mL to about 100 mg/mL (e.g., about 18 mg/mL to about 22 mg/mL, or about 20 mg/mL) of a G-CSF dimer (e.g., G-CSF-Fc dimer, such as efbemalenograstim alfa); (b) about 1 mM to about 50 mM sodium acetate; (c) about 0.1 mM to about 20 mM EDTA; (d) about 1% (w/v) to about 10% (w/v) sorbitol; and (e) about 0.001% (w/v) to about 0.1% (w/v) polysorbate 20, wherein the pharmaceutical composition has a pH of about 4.2 to about 6.2 (such as about 5.0 to about 5.4, or about 5.2). In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 50 mM sodium acetate; (c) about 0.1 mM to about 20 mM EDTA; (d) about 1% (w/v) to about 15% (w/v) sucrose; and (e) about 0.001% (w/v) to about 0.1% (w/v) polysorbate 20, wherein the pharmaceutical composition has a pH of about 4.2 to about 6.2 (such as about 5.0 to about 5.4, or about 5.2). In some embodiments, there is provided a pharmaceutical composition comprising (or consisting essentially of, or consisting of): (a) about 1 mg/mL to about 100 mg/mL (e.g., about 18 mg/mL to about 22 mg/mL, or about 20 mg/mL) of a G-CSF dimer (e.g., G-CSF-Fc dimer, such as efbemalenograstim alfa); (b) about 1 mM to about 50 mM sodium acetate; (c) about 0.1 mM to about 20 mM EDTA; (d) about 1% (w/v) to about 15% (w/v) sucrose; and (e) about 0.001% (w/v) to about 0.1% (w/v) polysorbate 20, wherein the pharmaceutical composition has a pH of about 4.2 to about 6.2 (such as about 5.0 to about 5.4, or about 5.2). In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile.

In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 30 mM sodium acetate; (c) about 0.1 mM to about 10 mM EDTA; (d) about 1% (w/v) to about 8% (w/v) sorbitol; and (e) about 0.005% (w/v) to about 0.05% (w/v) polysorbate 20, wherein the pharmaceutical composition has a pH of about 4.8 to about 5.8 (such as about 5.0 to about 5.4, or about 5.2). In some embodiments, there is provided a pharmaceutical composition comprising (or consisting essentially of, or consisting of): (a) about 5 mg/mL to about 50 mg/mL (e.g., about 20 mg/mL) of a G-CSF dimer (e.g., G-CSF-Fc dimer, such as efbemalenograstim alfa); (b) about 1 mM to about 30 mM sodium acetate; (c) about 0.1 mM to about 10 mM EDTA; (d) about 1% (w/v) to about 8% (w/v) sorbitol; and (e) about 0.005% (w/v) to about 0.05% (w/v) polysorbate 20, wherein the pharmaceutical composition has a pH of about 4.8 to about 5.8 (such as about 5.0 to about 5.4, or about 5.2). In some embodiments, there is provided a pharmaceutical composition comprising (or consisting essentially of, or consisting of): (a) a G-CSF dimer (e.g., G-CSF-Fc dimer, such as efbemalenograstim alfa); (b) about 1 mM to about 30 mM sodium acetate; (c) about 0.1 mM to about 10 mM EDTA; (d) about 5% (w/v) to about 15% (w/v) sucrose; and (e) about 0.005% (w/v) to about 0.05% (w/v) polysorbate 20, wherein the pharmaceutical composition has a pH of about 4.8 to about 5.8 (such as about 5.0 to about 5.4, or about 5.2). In some embodiments, there is provided a pharmaceutical composition comprising (or consisting essentially of, or consisting of): (a) about 5 mg/mL to about 50 mg/mL (e.g., about 20 mg/mL) of a G-CSF dimer (e.g., G-CSF-Fc dimer, such as efbemalenograstim alfa); (b) about 1 mM to about 30 mM sodium acetate; (c) about 0.1 mM to about 10 mM EDTA; (d) about 5% (w/v) to about 15% (w/v) sucrose; and (e) about 0.005% (w/v) to about 0.05% (w/v) polysorbate 20, wherein the pharmaceutical composition has a pH of about 4.8 to about 5.8 (such as about 5.0 to about 5.4, or about 5.2). In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile.

In some embodiments, there is provided a pharmaceutical composition comprising (or consisting essentially of, or consisting of): (a) a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20, wherein the pharmaceutical composition has a pH of about 4.2 to about 6.2 (such as about 5.0 to about 5.4, or about 5.2). In some embodiments, there is provided a pharmaceutical composition comprising (or consisting essentially of, or consisting of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20, wherein the pharmaceutical composition has a pH of about 4.2 to about 6.2 (such as about 5.0 to about 5.4, or about 5.2). In some embodiments, there is provided a pharmaceutical composition comprising (or consisting essentially of, or consisting of): (a) a G-CSF dimer (e.g., G-CSF-Fc dimer, such as efbemalenograstim alfa); (b) about 10 mM sodium acetate; (c) about 1 mM EDTA; (d) about 9% (w/v) sucrose; and (e) about 0.01% (w/v) polysorbate 20, wherein the pharmaceutical composition has a pH of about 4.2 to about 6.2 (such as about 5.0 to about 5.4, or about 5.2). In some embodiments, there is provided a pharmaceutical composition comprising (or consisting essentially of, or consisting of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 9% (w/v) sucrose; and (e) about 0.01% (w/v) polysorbate 20, wherein the pharmaceutical composition has a pH of about 4.2 to about 6.2 (such as about 5.0 to about 5.4, or about 5.2). In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile.

In some embodiments, there is provided a pharmaceutical composition comprising (or consisting essentially of, or consisting of): (a) a G-CSF dimer (e.g., G-CSF-Fc dimer, such as efbemalenograstim alfa); (b) about 10 mM sodium acetate; (c) about 1 mM EDTA; (d) about 9% (w/v) sucrose; and (e) about 0.01% (w/v) polysorbate 20, wherein the pharmaceutical composition has a pH of about 5.0 to about 5.4 (e.g., about 5.2). In some embodiments, there is provided a pharmaceutical composition comprising (or consisting essentially of, or consisting of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 9% (w/v) sucrose; and (e) about 0.01% (w/v) polysorbate 20, wherein the pharmaceutical composition has a pH of about 5.0 to about 5.4 (e.g., about 5.2). In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, there is provided a pharmaceutical composition comprising (or consisting essentially of, or consisting of): (a) efbemalenograstim alfa; (b) about 10 mM sodium acetate; (c) about 1 mM EDTA; (d) about 9% (w/v) sucrose; and (e) about 0.01% (w/v) polysorbate 20, wherein the pharmaceutical composition has a pH of about 5.0 to about 5.4 (e.g., about 5.2). In some embodiments, there is provided a pharmaceutical composition comprising (or consisting essentially of, or consisting of): (a) about 20 mg/mL of efbemalenograstim alfa; (b) about 10 mM sodium acetate; (c) about 1 mM EDTA; (d) about 9% (w/v) sucrose; and (e) about 0.01% (w/v) polysorbate 20, wherein the pharmaceutical composition has a pH of about 5.0 to about 5.4 (e.g., about 5.2). In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile.

In some embodiments, there is provided a pharmaceutical composition comprising (or consisting essentially of, or consisting of): (a) a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20, wherein the pharmaceutical composition has a pH of about 5.0 to about 5.4 (e.g., about 5.2). In some embodiments, there is provided a pharmaceutical composition comprising (or consisting essentially of, or consisting of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20, wherein the pharmaceutical composition has a pH of about 5.0 to about 5.4 (e.g., about 5.2). In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, there is provided a pharmaceutical composition comprising (or consisting essentially of, or consisting of): (a) efbemalenograstim alfa; (b) about 10 mM sodium acetate; (c) about 1 mM EDTA; (d) about 5% (w/v) sorbitol; and (e) about 0.01% (w/v) polysorbate 20, wherein the pharmaceutical composition has a pH of about 5.0 to about 5.4 (e.g., about 5.2). In some embodiments, there is provided a pharmaceutical composition comprising (or consisting essentially of, or consisting of): (a) about 20 mg/mL of efbemalenograstim alfa; (b) about 10 mM sodium acetate; (c) about 1 mM EDTA; (d) about 5% (w/v) sorbitol; and (e) about 0.01% (w/v) polysorbate 20, wherein the pharmaceutical composition has a pH of about 5.0 to about 5.4 (e.g., about 5.2). In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile.

In some embodiments, there is provided a pharmaceutical composition comprising (or consisting essentially of, or consisting of): (a) about 20 mg/mL of efbemalenograstim alfa; (b) about 10 mM sodium acetate; (c) about 1 mM EDTA; (d) about 5% (w/v) sorbitol; and (e) about 0.01% (w/v) polysorbate 20, wherein the pharmaceutical composition has a pH of about 5.2. Such pharmaceutical composition is also herein referred to as “efbemalenograstim alfa pharmaceutical composition.” In some embodiments, there is provided a pharmaceutical composition comprising (or consisting essentially of, or consisting of): (a) 20 mg/mL of efbemalenograstim alfa; (b) 10 mM sodium acetate; (c) 1 mM EDTA; (d) 5% (w/v) sorbitol; and (e) 0.01% (w/v) polysorbate 20, wherein the pharmaceutical composition has a pH of 5.2. In some embodiments, the pharmaceutical composition is contained in a syringe. In some embodiments, the volume of the pharmaceutical composition within the syringe is about 1 mL. In some embodiments, the syringe is for single use. In some embodiments, the syringe is sterile. In some embodiments, the pharmaceutical composition is a clear, colorless, preservative-free solution supplied in a prefilled single-dose syringe with a 27-gauge, ½-inch needle and an UltraSafe Passive™ Needle Guard, containing 20 mg of efbemalenograstim alfa. In some embodiments, each syringe contains about 20 mg efbemalenograstim alfa in a sterile, clear, colorless, preservative-free solution (about pH 5.2) containing acetate (about 0.6 mg), EDTA (about 0.29 mg), polysorbate 20 (about 0.1 mg), sodium (about 0.23 mg), and sorbitol (about 50 mg) in water for injection. In some embodiments, each syringe contains 20 mg efbemalenograstim alfa in a sterile, clear, colorless, preservative-free solution (pH 5.2) containing acetate (0.6 mg), EDTA (0.29 mg), polysorbate 20 (0.1 mg), sodium (0.23 mg), and sorbitol (50 mg) in water for injection.

In some embodiments, there is provided a pharmaceutical composition comprising (or consisting essentially of, or consisting of): (a) a G-CSF dimer (e.g., G-CSF-Fc dimer, such as efbemalenograstim alfa); (b) sodium; (c) acetate; (d) EDTA; (e) sorbitol; and (f) polysorbate 20 or polysorbate 80 (e.g., polysorbate 20). In some embodiments, there is provided a pharmaceutical composition comprising (or consisting essentially of, or consisting of): (a) a G-CSF dimer (e.g., G-CSF-Fc dimer, such as efbemalenograstim alfa); (b) sodium; (c) acetate; (d) EDTA; (e) sucrose; and (f) polysorbate 20 or polysorbate 80 (e.g., polysorbate 20). In some embodiments, there is provided a pharmaceutical composition comprising (or consisting essentially of, or consisting of): (a) a G-CSF dimer (e.g., G-CSF-Fc dimer, such as efbemalenograstim alfa); (b) sodium acetate; (c) EDTA; (d) sorbitol; and (e) polysorbate 20 or polysorbate 80 (e.g., polysorbate 20). In some embodiments, there is provided a pharmaceutical composition comprising (or consisting essentially of, or consisting of): (a) a G-CSF dimer (e.g., G-CSF-Fc dimer, such as efbemalenograstim alfa); (b) sodium acetate; (c) EDTA; (d) sucrose; and (e) polysorbate 20 or polysorbate 80 (e.g., polysorbate 20). In some embodiments, the pharmaceutical composition has a pH of about 4.2 to about 6.2 (such as about 5.0 to about 5.4, or about 5.2). In some embodiments, the pharmaceutical composition further comprises water. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition comprises (or consists essentially of, or consists of) about 20 mg efbemalenograstim alfa, about 0.6 mg acetate, about 0.29 mg EDTA, about 0.1 mg polysorbate 20, about 0.23 mg sodium, and about 50 mg sorbitol. In some embodiments, the pharmaceutical composition is lyophilized. In some embodiments, the pharmaceutical composition is in about 1 mL solution (e.g., in water). In some embodiments, the pharmaceutical composition has a pH of about 5.2.

In some embodiments, there is also provided a syringe comprising any of the pharmaceutical compositions described herein (e.g., G-CSF-Fc dimer pharmaceutical composition, such as efbemalenograstim alfa pharmaceutical composition). In some embodiments, the volume of the pharmaceutical composition within the syringe is about 1 mL. In some embodiments, the syringe is for single use. In some embodiments, the syringe is sterile. In some embodiments, the prefilled syringe does not bear graduation marks and is intended only to deliver the entire contents of the syringe (20 mg/1 mL) for direct administration to an individual (e.g., a human adult). In some embodiments, the needle cap of the prefilled syringe contains natural rubber latex.

### Properties of the Pharmaceutical Compositions

In some embodiments, the pharmaceutical composition has a purity of G-CSF dimer (e.g., G-CSF-Fc dimer, such as efbemalenograstim alfa) of at least about 90% (e.g., at least about any of 91%, 92%, 93%, 94%, 95%, 96%, 97%, 98%, 99%, or more) as assessed by SE-HPLC. In some embodiments, the purity is measured before storage. In some embodiments, the purity is measured after storage. In some embodiments, the storage condition comprises storing at about 2° C. to about 8° C. for at least about 36 months. In some embodiments, the storage condition comprises storing at 25±2° C. for at least about 1 month (e.g., at least about 2 months). In some embodiments, the storage condition comprises storing at about 2° C. to about 8° C. up to about 36 months. In some embodiments, the storage condition comprises storing at 25±2° C. up to about 3 months.

The presently described pharmaceutical compositions in some embodiments are characterized by a prolonged stability. In some embodiments, the pharmaceutical composition comprising a G-CSF dimer (e.g., G-CSF-Fc dimer, such as efbemalenograstim alfa) is stable at about 2° C. to about 8° C. for at least about any of 1 week, 2 weeks, 3 weeks, 4 weeks, 2 months, 3 months, 4 months, 5 months, 6 months, 7 months, 8 months, 12 months, 18 months, 24 months, 30 months, 36 months, 48 months, 54 months, 60 months, or longer. In some embodiments, the pharmaceutical composition comprising a G-CSF dimer (e.g., G-CSF-Fc dimer, such as efbemalenograstim alfa) is stable at about 2° C. to about 8° C. up to about 36 months. In some embodiments, the pharmaceutical composition is stable at 25±2° C. for at least about any of 24 hours, 48 hours, 3 days, 4 days, 5 days, 6 days, 1 week, 2 weeks, 3 weeks, 1 month, 2 months, 3 months, 4 months, 5 months, 6 months, or longer. In some embodiments, the pharmaceutical composition is stable at 25±2° C. up to about 6 months, such as up to about 3 months (e.g., stable after 3, 2, or 1 month(s) of storage). In some embodiments, the stability is assessed upon storage of the liquid pharmaceutical composition under suitable storage conditions. In some embodiments, the stability is assessed on a liquid pharmaceutical composition reconstituted from a lyophilized pharmaceutical composition upon storage of the lyophilized pharmaceutical composition under suitable storage conditions. In some embodiments, the stability is assessed on a liquid pharmaceutical composition reconstituted from a frozen pharmaceutical composition upon storage of the frozen pharmaceutical composition under suitable storage conditions.

In some embodiments, the storage condition comprises storing at about 2° C. to about 8° C. In some embodiments, the storage condition comprises storing at 25±2° C. In some embodiments, the storage condition comprises storing for least about any of 5 minutes, 30 minutes, 1 hour, 2 hours, 4 hours, 8 hours, 12 hours, 24 hours, 48 hours, 3 days, 4 days, 5 days, 6 days, 1 week, 2 weeks, 3 weeks, 1 month, 2 months, 3 months, 4 months, 5 months, 6 months, 7 months, 8 months, 12 months, 18 months, 24 months, 30 months, 36 months, 48 months, 54 months, 60 months, or longer. In some embodiments, the storage condition comprises storing up to about 36 months, such as up to about 6 months, or up to about 3 moths. In some embodiments, the storage condition comprises storing at about 2° C. to about 8° C. up to about 36 months. In some embodiments, the storage condition comprises storing at 25±2° C. up to about 3 months. In some embodiments, the storage condition comprises avoiding light. In some embodiments, the storage condition comprises avoiding shaking. In some embodiments, the storage condition comprises avoiding freezing. In some embodiments, the storage condition comprises freezing, and then thawing at about 2° C. to about 8° C. before using. In some embodiments, the storage condition comprises avoiding freezing more than once. In some embodiments, the storage condition comprises storing under 25±2° C. for no more than about 48 hours.

In some embodiments, the pharmaceutical composition (e.g., G-CSF-Fc dimer pharmaceutical composition, such as efbemalenograstim alfa pharmaceutical composition) has superior freeze-thaw stability, such as being stable after at least 6 (e.g., 6, 7, 8, or more) cycles of freezing at about −20° C. and thawing at room temperature.

In some embodiments, the pharmaceutical composition (e.g., G-CSF-Fc dimer pharmaceutical composition, such as efbemalenograstim alfa pharmaceutical composition) is within the clinically acceptance range after storage. In some embodiments, the pharmaceutical composition comprises at least about 96% (such as at least about any of 97%, 98%, 99%, or 100%) of G-CSF dimer as measured by size exclusion chromatography (SEC) after storage. In some embodiments, the pharmaceutical composition comprises at least about 95.5% (such as at least about any of 96%, 97%, 98%, 99%, or 100%) of G-CSF dimer as measured by rCE-SDS after storage. In some embodiments, the pharmaceutical composition comprises at least about 94% (such as at least about any of 95%, 96%, 97%, 98%, 99%, or 100%) of G-CSF dimer as measured by nrCE-SDS after storage. In some embodiments, the pharmaceutical composition comprises no more than about 1.5% (such as no more than about any of 1.4%, 1.3%, 1.2%, 1.1%, 1.0%, 0.8%, 0.6%, 0.4%, 0.1%, or less) of HMWS as measured by rCE-SDS after storage. In some embodiments, the pharmaceutical composition comprises no more than about 1.5% (such as no more than about any of 1.4%, 1.3%, 1.2%, 1.1%, 1.0%, 0.8%, 0.6%, 0.4%, 0.1%, or less) of HMWS as measured by nrCE-SDS after storage. In some embodiments, the pharmaceutical composition comprises about 27.2% to about 39.0% (e.g., about 30% to about 40%, such as about 32% to about 37%) acidic region, about 36.5% to about 60.1% (e.g., about 45% to about 55%, such as about 47% to about 55%) main peak, and about 7.2% to about 30% (e.g., about 10% to about 20%, such as about 11% to 18%) basic region as measured by icIEF after storage. In some embodiments, the pharmaceutical composition retains at least about 70% (e.g., at least about any of 75%, 80%, 85%, 90%, 95%, 99%, or 100%) potency (e.g., potency of G-CSF) after storage. In some embodiments, the pharmaceutical composition retains about 70% to about 130% (e.g., about any of 75%, 80%, 85%, 90%, 95%, 100%, 110%, 120%, or 130%) potency (e.g., potency of G-CSF), such as by cell proliferation assay, after storage, In some embodiments, the concentration (e.g., G-CSF dimer concentration) of the pharmaceutical composition varies by less than about 0.5 mg/mL (e.g., less than about any of 0.4 mg/mL, 0.3 mg/mL, 0.2 mg/mL, 0.1 mg/mL, 0.05 mg/mL, or less) after storage. In some embodiments, the storage condition comprises storing at about 2° C. to about 8° C. for at least about 36 months. In some embodiments, the storage condition comprises storing at 25±2° C. for at least about 1 month (e.g., at least about 2 months). In some embodiments, the storage condition comprises storing at about 2° C. to about 8° C. up to about 36 months. In some embodiments, the storage condition comprises storing at 25±2° C. up to about 3 months.

In some embodiments, the potency of a G-CSF molecule (e.g., G-CSF dimer, such as G-CSF-Fc dimer) or a pharmaceutical composition thereof comprises one or more of: i) promoting (e.g., at least about any of 10%, 20%, 50%, 1-fold, 2-fold, 10-fold, 20-fold, or more) the growth and/or differentiation of blood cells (e.g., neutrophils); ii) enhancing (e.g., at least about any of 10%, 20%, 50%, 1-fold, 2-fold, 10-fold, 20-fold, or more) immune response (e.g., defense against bacterial infections); iii) activating mature neutrophils (e.g., to participate in immune response, or to synergize with other hematopoietic growth factors such as stem cell factor, Flt-3 ligand, and GM-CSF to perform hematopoietic functions); iv) improving (e.g., at least about any of 10%, 20%, 50%, 1-fold, 2-fold, 10-fold, 20-fold, or more) hematopoietic function; v) increasing (e.g., at least about any of 10%, 20%, 50%, 1-fold, 2-fold, 10-fold, 20-fold, or more) neutrophil count; vi) promoting (e.g., at least about any of 10%, 20%, 50%, 1-fold, 2-fold, 10-fold, 20-fold, or more) neutrophil mobilization; and vii) reducing (e.g., at least about any of 10%, 20%, 30%, 40%, 50%, 60%, 70%, 80%, 90%, or 100%) mobilization of peripheral blood progenitor cells.

In some embodiments, the pharmaceutical composition (e.g., liquid pharmaceutical composition) has an appearance with one or more properties: i) clear, ii) colorless to slightly yellowish, and iii) essentially free (e.g., no more than about 10%, such as no more than about any of 8%, 5%, 2%, 1%, or less) of visible particulates. The appearance can be before and/or after storage. In some embodiments, the storage condition comprises storing at about 2° C. to about 8° C. up to about 36 months. In some embodiments, the storage condition comprises storing at 25±2° C. up to about 3 months. In some embodiments, the pharmaceutical composition is placed into colorimetric cuvettes and examined by eye against a black and a white background, respectively, for visible particles. In some embodiments, the pharmaceutical composition is compared to colorimetric standards against a white background to evaluate color.

In some embodiments, the pharmaceutical composition is characterized by less and/or smaller particles. In some embodiments, the pharmaceutical composition comprises particles with ≥10 μm diameter of ≤6000 per vial and particles with ≥25 μm diameter of ≤600 per vial. In some embodiments, the pharmaceutical composition is transferred into a vial, and the particles in the vial are examined using a particle sizing system (e.g., ACCUSIZER 780 SIS). The particulate property of the pharmaceutical composition can be before and/or after storage. In some embodiments, the storage condition comprises storing at about 2° C. to about 8° C. up to about 36 months. In some embodiments, the storage condition comprises storing at 25±2° C. up to about 3 months.

In some embodiments, the pharmaceutical compositions described herein (e.g., G-CSF-Fc dimer pharmaceutical composition, such as efbemalenograstim alfa pharmaceutical composition) are characterized by low viscosity. In some embodiments, the viscosity of the pharmaceutical composition ranges from about 1 to about 50 mPa-S, such as any of about 1 to about 45 mPa-S, about 1 to about 40 mPa-S, about 1 to about 35 mPa-S, about 1 to about 30 mPa-S, about 1 to about 25 mPa-S, or about 1 to about 20 mPa-S, about 1 to about 15 mPa-S, about 1 to about 10 mPa-S, about 1 to about 5 mPa-S, about 1 to about 4 mPa-S, about 1 to about 3 mPa-S, or about 1 to about 2 mPa-S, at between about 5° C. to about 25° C. For example, in some embodiments, the pharmaceutical composition has a viscosity of about 1-2 mPa-S at about 25° C. In some embodiments, the pharmaceutical composition is isotonic. The viscosity property of the pharmaceutical composition can be before and/or after storage. In some embodiments, the storage condition comprises storing at about 2° C. to about 8° C. up to about 36 months. In some embodiments, the storage condition comprises storing at 25±2° C. up to about 3 months.

In some embodiments, the osmolality of the pharmaceutical compositions described herein (e.g., G-CSF-Fc dimer pharmaceutical composition, such as efbemalenograstim alfa pharmaceutical composition) ranges from about 100 to about 500 mOsm/kg, such as any of about 120 to about 480 mOsm/kg, about 140 to about 440 mOsm/kg, about 160 to about 420 mOsm/kg, about 180 to about 400 mOsm/kg, about 200 to about 380 mOsm/kg, about 220 to about 360 mOsm/kg, about 270 to about 350 mOsm/kg, or about 268 to about 360 mOsm/kg. In some embodiments, the osmolality of the pharmaceutical composition is about 310 mOsm/kg. The osmolality property of the pharmaceutical composition can be before and/or after storage. In some embodiments, the storage condition comprises storing at about 2° C. to about 8° C. up to about 36 months. In some embodiments, the storage condition comprises storing at 25±2° C. up to about 3 months.

In some embodiments, the pharmaceutical compositions described herein (e.g., G-CSF-Fc dimer pharmaceutical composition, such as efbemalenograstim alfa pharmaceutical composition) are characterized by low percentage of protein aggregation (e.g., before and/or after storage). In some embodiments, the percentage of total protein aggregation is less than about 5%, such as less than about any of 4%, 13%, 2%, 1%, 0.5%, 0.2%, 0.1%, or less. In some embodiments, the percentage of total protein aggregation is less than about 2% as measured by SEC. In some embodiments, the storage condition comprises storing at about 2° C. to about 8° C. for at least about 36 months. In some embodiments, the storage condition comprises storing at 25±2° C. for at least about 1 month (e.g., at least about 2 months). In some embodiments, the storage condition comprises storing at about 2° C. to about 8° C. up to about 36 months. In some embodiments, the storage condition comprises storing at 25±2° C. up to about 3 months.

In some embodiments, the pharmaceutical composition (e.g., G-CSF-Fc dimer pharmaceutical composition, such as efbemalenograstim alfa pharmaceutical composition) comprises no more than about 1.5% (e.g., no more than about any of 1.4%, 1.3%, 1.2%, 1.1, 1%, 0.8%, 0.5%, 0.1%, or less, such as no more than about 1%) HMWS as measured by rCE-SDS after at least about 1 month of storage under 25±2° C. In some embodiments, the pharmaceutical formulation comprises no more than about 1.5% (e.g., no more than about any of 1.4%, 1.3%, 1.2%, 1.1, 1%, 0.8%, 0.5%, 0.1%, or less, such as no more than about 1%) HMWS as measured by rCE-SDS after at least about 2 months of storage under 25±2° C. In some embodiments, the pharmaceutical formulation comprises no more than about 1.5% HMWS as measured by rCE-SDS after up to about 3 months of storage under 25±2° C. In some embodiments, the pharmaceutical composition comprises no more than about 1.5% (e.g., no more than about any of 1.4%, 1.3%, 1.2%, 1.1, 1%, 0.8%, 0.5%, 0.1%, or less) HMWS as measured by rCE-SDS after at least about 36 months of storage under about 2° C. to about 8° C. In some embodiments, the pharmaceutical composition comprises no more than about 1.5% HMWS as measured by rCE-SDS after up to about 36 months of storage under about 2° C. to about 8° C. In some embodiments, the pharmaceutical composition comprises no more than about 1.5% (e.g., no more than about any of 1.4%, 1.3%, 1.2%, 1.1, 1%, 0.8%, 0.5%, 0.1%, or less) HMWS as measured by nrCE-SDS after at least about 1 month of storage under 25±2° C. In some embodiments, the pharmaceutical formulation comprises no more than about 1.5% (e.g., no more than about any of 1.4%, 1.3%, 1.2%, 1.1, 1%, 0.8%, 0.5%, 0.1%, or less) HMWS as measured by nrCE-SDS after at least about 2 months of storage under 25±2° C. In some embodiments, the pharmaceutical formulation comprises no more than about 1.5% HMWS as measured by nrCE-SDS after up to about 3 months of storage under 25±2° C. In some embodiments, the pharmaceutical composition comprises no more than about 1.5% (e.g., no more than about any of 1.4%, 1.3%, 1.2%, 1.1, 1%, 0.8%, 0.5%, 0.1%, or less) HMWS as measured by nrCE-SDS after at least about 36 months of storage under about 2° C. to about 8° C. In some embodiments, the pharmaceutical composition comprises no more than about 1.5% HMWS as measured by nrCE-SDS after up to about 36 months of storage under about 2° C. to about 8° C.

In some embodiments, the pharmaceutical composition (e.g., G-CSF-Fc dimer pharmaceutical composition, such as efbemalenograstim alfa pharmaceutical composition) comprises at least about 90% (such as at least about any of 91%, 92%, 93%, 94%, 95%, 96%, 97%, 98%, 99%, or more) of the G-CSF dimer after storage. In some embodiments, the pharmaceutical composition comprises at least about 90% (e.g., at least about any of 91%, 92%, 93%, 94%, 95%, 96%, 97%, 98%, 99%, or 100%) of the G-CSF dimer, such as at least about 95.5% or at least about 96% of the G-CSF dimer, as measured by rCE-SDS after at least about 1 month of storage under 25±2° C. In some embodiments, the pharmaceutical composition comprises at least about 90% (e.g., at least about any of 91%, 92%, 93%, 94%, 95%, 96%, 97%, 98%, 99%, or 100%) of the G-CSF dimer, such as at least about 95.5% or at least about 96% of the G-CSF dimer, as measured by rCE-SDS after at least about 2 months of storage under 25±2° C. In some embodiments, the pharmaceutical composition comprises at least about 90% of the G-CSF dimer, such as at least about 95.5% or at least about 96% of the G-CSF dimer, as measured by rCE-SDS after up to about 3 months of storage under 25±2° C. In some embodiments, the pharmaceutical composition comprises at least about 90% (e.g., at least about any of 91%, 92%, 93%, 94%, 95%, 96%, 97%, 98%, 99%, or 100%) of the G-CSF dimer, such as at least about 95.5% of the G-CSF dimer, as measured by rCE-SDS after at least about 36 months of storage under about 2° C. to about 8° C. In some embodiments, the pharmaceutical composition comprises at least about 90% of the G-CSF dimer, such as at least about 95.5% of the G-CSF dimer, as measured by rCE-SDS after up to about 36 months of storage under about 2° C. to about 8° C. In some embodiments, the pharmaceutical composition comprises at least about 90% (e.g., at least about any of 91%, 92%, 93%, 94%, 95%, 96%, 97%, 98%, 99%, or 100%) of the G-CSF dimer, such as at least about 94% of the G-CSF dimer, as measured by nrCE-SDS after at least about 1 month of storage under 25±2° C. In some embodiments, the pharmaceutical composition comprises at least about 90% (e.g., at least about any of 91%, 92%, 93%, 94%, 95%, 96%, 97%, 98%, 99%, or 100%) of the G-CSF dimer, such as at least about 94% of the G-CSF dimer, as measured by nrCE-SDS after at least about 2 months of storage under 25±2° C. In some embodiments, the pharmaceutical composition comprises at least about 90% of the G-CSF dimer, such as at least about 94% of the G-CSF dimer, as measured by nrCE-SDS after up to about 3 months of storage under 25±2° C. In some embodiments, the pharmaceutical composition comprises at least about 90% (e.g., at least about any of 91%, 92%, 93%, 94%, 95%, 96%, 97%, 98%, 99%, or 100%) of the G-CSF dimer, such as at least about 94% of G-CSF dimer, as measured by nrCE-SDS after at least about 36 months of storage under about 2° C. to about 8° C. In some embodiments, the pharmaceutical composition comprises at least about 90% of the G-CSF dimer, such as at least about 94% of G-CSF dimer, as measured by nrCE-SDS after up to about 36 months of storage under about 2° C. to about 8° C.

In some embodiments, the pharmaceutical composition (e.g., G-CSF-Fc dimer pharmaceutical composition, such as efbemalenograstim alfa pharmaceutical composition) comprises at least about 90% (e.g., at least about any of 91%, 92%, 93%, 94%, 95%, 96%, 97%, 98%, 99%, or 100%) of the G-CSF dimer, such as at least about 97% of the G-CSF dimer, as measured by SEC. In some embodiments, the pharmaceutical composition comprises a main peak at least about 85% (e.g., at least about any of 86%, 87%, 88%, 89%, 90%, 91%, 92%, 93%, 94%, 95%, 96%, 97%, 98%, 99%, or more) in relation to the total area of all peaks when measured by SE-HPLC after at least about 1 month of storage under 25±2° C. In some embodiments, the pharmaceutical composition comprises a main peak at least about 85% (e.g., at least about any of 86%, 87%, 88%, 89%, 90%, 91%, 92%, 93%, 94%, 95%, 96%, 97%, 98%, 99%, or more) in relation to the total area of all peaks when measured by SE-HPLC after at least about 2 months of storage under 25±2° C. In some embodiments, the pharmaceutical composition comprises a main peak at least about 85% in relation to the total area of all peaks when measured by SE-HPLC after up to about 3 months of storage under 25±2° C. In some embodiments, the pharmaceutical composition comprises a main peak at least about 85% (e.g., at least about any of 86%, 87%, 88%, 89%, 90%, 91%, 92%, 93%, 94%, 95%, 96%, 97%, 98%, 99%, or more) in relation to the total area of all peaks when measured by SE-HPLC after at least about 36 months of storage under about 2° C. to about 8° C. In some embodiments, the pharmaceutical composition comprises a main peak at least about 85% in relation to the total area of all peaks when measured by SE-HPLC after up to about 36 months of storage under about 2° C. to about 8° C.

In some embodiments, the pharmaceutical composition (e.g., G-CSF-Fc dimer pharmaceutical composition, such as efbemalenograstim alfa pharmaceutical composition) comprises a dimer less than about 5% (e.g., less than about any of 4.5%, 4%, 3.5%, 3%, 2.5%, 2%, 1.5%, 1%, 0.5%, 0.1%, or less, such as less than about 2.5%) in relation to the total area of all peaks when measured by SE-HPLC after at least about 1 month of storage under 25±2° C. In some embodiments, the pharmaceutical composition comprises a dimer less than about 5% (e.g., less than about any of 4.5%, 4%, 3.5%, 3%, 2.5%, 2%, 1.5%, 1%, 0.5%, 0.1%, or less, such as less than about 2.5%) in relation to the total area of all peaks when measured by SE-HPLC after at least about 2 months of storage under 25±2° C. In some embodiments, the pharmaceutical composition comprises a dimer less than about 5% (such as less than about 2.5%) in relation to the total area of all peaks when measured by SE-HPLC after up to about 3 months of storage under 25±2° C. In some embodiments, the pharmaceutical composition comprises a dimer less than about 5% (e.g., less than about any of 4.5%, 4%, 3.5%, 3%, 2.5%, 2%, 1.5%, 1%, 0.5%, 0.1%, or less, such as less than about 2.5%) in relation to the total area of all peaks when measured by SE-HPLC after at least about 36 months of storage under about 2° C. to about 8° C. In some embodiments, the pharmaceutical composition comprises a dimer less than about 5% (such as less than about 2.5%) in relation to the total area of all peaks when measured by SE-HPLC after up to about 36 months of storage under about 2° C. to about 8° C.

In some embodiments, the pharmaceutical composition (e.g., G-CSF-Fc dimer pharmaceutical composition, such as efbemalenograstim alfa pharmaceutical composition) comprises an aggregate in no more than about 2% (e.g., no more than about any of 1.8%, 1.5%, 1%, 0.5%, 0.1%, or less) in relation to the total area of all peaks when measured by SE-HPLC after at least about 1 month of storage under 25±2° C. In some embodiments, the pharmaceutical composition comprises an aggregate in no more than about 2% (e.g., no more than about any of 1.8%, 1.5%, 1%, 0.5%, 0.1%, or less) in relation to the total area of all peaks when measured by SE-HPLC after at least about 2 months of storage under 25±2° C. In some embodiments, the pharmaceutical composition comprises an aggregate in no more than about 2% in relation to the total area of all peaks when measured by SE-HPLC after up to about 3 months of storage under 25±2° C. In some embodiments, the pharmaceutical composition comprises an aggregate in no more than about 2% (e.g., no more than about any of 1.8%, 1.5%, 1%, 0.5%, 0.1%, or less) in relation to the total area of all peaks when measured by SE-HPLC after at least about 36 months of storage under about 2° C. to about 8° C. In some embodiments, the pharmaceutical composition comprises an aggregate in no more than about 2% in relation to the total area of all peaks when measured by SE-HPLC after up to about 36 months of storage under about 2° C. to about 8° C.

In some embodiments, the pharmaceutical composition (e.g., liquid pharmaceutical composition) (e.g., G-CSF-Fc dimer pharmaceutical composition, such as efbemalenograstim alfa pharmaceutical composition) comprises about 27.2% to about 39.0% (e.g., about 30% to about 40%, such as about 32% to about 37%) acidic region, about 36.5% to about 60.1% (e.g., about 45% to about 55%, such as about 47% to about 55%) main peak, and about 7.2% to about 30% (e.g., about 10% to about 20%, such as about 11% to 18%) basic region as measured by icIEF following storage at 25±2° C. for at least about 1 month. In some embodiments, the pharmaceutical composition (e.g., liquid pharmaceutical composition) comprises about 27.2% to about 39.0% (e.g., about 30% to about 40%, such as about 32% to about 37%) acidic region, about 36.5% to about 60.1% (e.g., about 45% to about 55%, such as about 47% to about 55%) main peak, and about 7.2% to about 30% (e.g., about 10% to about 20%, such as about 11% to 18%) basic region as measured by icIEF following storage at 25±2° C. for at least about 2 months. In some embodiments, the pharmaceutical composition (e.g., liquid pharmaceutical composition) comprises about 27.2% to about 39.0% acidic region, about 36.5% to about 60.1% main peak, and about 7.2% to about 30% basic region as measured by icIEF following storage at 25±2° C. up to about 3 months. In some embodiments, the pharmaceutical composition (e.g., liquid pharmaceutical composition) comprises about 27.2% to about 39.0% (e.g., about 30% to about 40%, such as about 32% to about 37%) acidic region, about 36.5% to about 60.1% (e.g., about 45% to about 55%, such as about 47% to about 55%) main peak, and about 7.2% to about 30% (e.g., about 10% to about 20%, such as about 11% to 18%) basic region as measured by icIEF following storage at about 2° C. to about 8° C. for at least about 36 month. In some embodiments, the pharmaceutical composition (e.g., liquid pharmaceutical composition) comprises about 27.2% to about 39.0% acidic region, about 36.5% to about 60.1% main peak, and about 7.2% to about 30% basic region as measured by icIEF following storage at about 2° C. to about 8° C. up to about 36 months.

In some embodiments, the pharmaceutical composition (e.g., G-CSF-Fc dimer pharmaceutical composition, such as efbemalenograstim alfa pharmaceutical composition) retains at least about 70% (e.g., at least about any of 75%, 80%, 85%, 90%, 95%, 99%, or 100%) potency (e.g., potency of G-CSF) after at least about 1 month of storage under 25±2° C. In some embodiments, the pharmaceutical composition retains at least about 70% (e.g., at least about any of 75%, 80%, 85%, 90%, 95%, 99%, or 100%) potency (e.g., potency of G-CSF) after at least about 2 months of storage under 25±2° C. In some embodiments, the pharmaceutical composition retains at least about 70% potency (e.g., potency of G-CSF) after up to about 3 months of storage under 25±2° C. In some embodiments, the pharmaceutical composition retains at least about 70% (e.g., at least about any of 75%, 80%, 85%, 90%, 95%, 99%, or 100%) potency (e.g., potency of G-CSF) after at least about 36 months of storage under about 2° C. to about 8° C. In some embodiments, the pharmaceutical composition retains at least about 70% potency (e.g., potency of G-CSF) after up to about 36 months of storage under about 2° C. to about 8° C.

In some embodiments, the concentration (e.g., G-CSF dimer concentration) of the pharmaceutical composition (e.g., G-CSF-Fc dimer pharmaceutical composition, such as efbemalenograstim alfa pharmaceutical composition) varies by less than about 0.5 mg/mL (e.g., less than about any of 0.4 mg/mL, 0.3 mg/mL, 0.2 mg/mL, 0.1 mg/mL, or less) after at least about 1 month of storage under 25±2° C. In some embodiments, the concentration (e.g., G-CSF dimer concentration) of the pharmaceutical composition varies by less than about 0.5 mg/mL (e.g., less than about any of 0.4 mg/mL, 0.3 mg/mL, 0.2 mg/mL, 0.1 mg/mL, or less) after at least about 2 months of storage under 25±2° C. In some embodiments, the concentration (e.g., G-CSF dimer concentration) of the pharmaceutical composition varies by less than about 0.5 mg/mL after up to about 3 months of storage under 25±2° C. In some embodiments, the concentration (e.g., G-CSF dimer concentration) of the pharmaceutical composition varies by less than about 0.5 mg/mL (e.g., less than about any of 0.4 mg/mL, 0.3 mg/mL, 0.2 mg/mL, 0.1 mg/mL, or less) after at least about 36 months of storage under about 2° C. to about 8° C. In some embodiments, the concentration (e.g., G-CSF dimer concentration) of the pharmaceutical composition varies by less than about 0.5 mg/mL after up to about 36 months of storage under about 2° C. to about 8° C.

In some embodiments, there is provided a commercial batch of any of the pharmaceutical compositions described herein (e.g., G-CSF-Fc dimer pharmaceutical composition, such as efbemalenograstim alfa pharmaceutical composition). “Commercial batch” used herein refers to a batch size that is at least about 20 grams. Commercial batches are produced at a larger scale than experimental or bench-scale batches. The increased scale is associated with longer production times, including longer steps (such as evaporation steps) or longer hold times between steps. In some embodiments, there is no more than about 5% (e.g., no more than about any of 4%, 3%, 2%, 1%, 0.5%, 0.1%, or less) variation in the pharmaceutical compositions among batches, such as variations in any of: the amount(s) of the pharmaceutical composition component(s), the pH of the pharmaceutical composition, the purity of the pharmaceutical composition, and the one or more properties of the pharmaceutical composition (such as composition, stability, viscosity, osmolality, purity, and/or potency before and/or after storage).

Also provided are methods of making any of the pharmaceutical compositions described herein (e.g., G-CSF-Fc dimer pharmaceutical composition, such as efbemalenograstim alfa pharmaceutical composition), comprising admixing the components of the pharmaceutical composition at the amounts described herein to make the pharmaceutical composition. In some embodiments, the pharmaceutical composition is a liquid pharmaceutical composition. In some embodiments, the pharmaceutical composition is a dry powder (such as a lyophilized pharmaceutical composition). In some embodiments, the pharmaceutical composition is a reconstituted pharmaceutical composition. In some embodiments, the method further comprises filling the pharmaceutical composition into a syringe (e.g., sterile syringe). In some embodiments, the method further comprises purifying the G-CSF molecule (e.g., G-CSF dimer) before admixing with other components of the pharmaceutical composition. In some embodiments, the method further comprises detecting (or validating) one or more properties of the pharmaceutical composition, such as composition, stability, viscosity, osmolality, purity, and/or potency before and/or after storage.

### III. Method of Validating Pharmaceutical Compositions

The present application in another aspect provides a method of assessing suitability of any of the pharmaceutical compositions described herein (e.g., G-CSF-Fc dimer pharmaceutical composition, such as efbemalenograstim alfa pharmaceutical composition) for medical use in an individual (e.g., a human individual, such as a human with cancer and/or neutropenia). Such validated pharmaceutical compositions are also herein referred to as “suitable for medical use.” In some embodiments, there is provided a method of assessing the suitability of a pharmaceutical composition for medical use in an individual (e.g., human, such as a human with cancer and/or neutropenia), wherein the pharmaceutical composition comprises (or consists essentially of, or consists of): (a) about 1 mg/mL to about 100 mg/mL G-CSF dimer (e.g., G-CSF-Fc dimer, such as efbemalenograstim alfa); (b) about 1 mM to about 50 mM buffering agent (e.g., sodium acetate); (c) about 0.1 mM to about 20 mM stabilizing agent (e.g., EDTA); (d) about 1% (w/v) to about 15% (w/v) (e.g., about 1% (w/v) to about 10% (w/v)) tonicity agent (e.g., sorbitol or sucrose); and (e) about 0.001% (w/v) to about 0.1% (w/v) surfactant (e.g., polysorbate 20), wherein the pH of the pharmaceutical composition is about 4.2 to about 6.2 (e.g., about 5.0 to about 5.4, such as about 5.2), the method comprising measuring a quality control parameter for the pharmaceutical composition; and assessing the suitability of the pharmaceutical composition for medical use in an individual, wherein a measured quality control parameter within a quality control parameter threshold is indicative of suitability of the pharmaceutical composition for medical use. In some embodiments, there is provided a method of assessing the suitability of a pharmaceutical composition for medical use in an individual (e.g., human, such as a human with cancer and/or neutropenia), wherein the pharmaceutical composition comprises (or consists essentially of, or consists of): (a) about 20 mg/mL G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20, wherein the pH of the pharmaceutical composition is about 5.2, the method comprising measuring a quality control parameter for the pharmaceutical composition; and assessing the suitability of the pharmaceutical composition for medical use in an individual, wherein a measured quality control parameter within a quality control parameter threshold is indicative of suitability of the pharmaceutical composition for medical use. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. The quality control parameter can be any one or a combination of multiple parameters described herein (e.g., composition, viscosity, stability, osmolality, purity, or potency) for the pharmaceutical composition, for example, as assessed by corresponding methods described herein. The one or more quality control parameters can be measured before storage and/or after storage. Any of the storage condition described above under “II. Pharmaceutical compositions” can be used herein for validation purpose. Also see Example 8 and Table 14 for one or more quality control parameters, acceptance criteria (e.g., quality control parameter threshold), and related methods.

In some embodiments, the quality control parameter comprises appearance of the pharmaceutical composition (e.g., before and/or after storage). In some embodiments, the appearance being clear, colorless to slightly yellowish solution, and essentially free (e.g., no more than about 10%, such as no more than about any of 8%, 5%, 2%, 1%, or less) of visible particulates is indicative of suitability of the pharmaceutical composition for medical use. In some embodiments, the pharmaceutical composition is placed into colorimetric cuvettes and examined by eye against a black and a white background, respectively, for visible particles. In some embodiments, the pharmaceutical composition is compared to colorimetric standards against a white background to evaluate color. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises pH of the pharmaceutical composition (e.g., before and/or after storage). In some embodiments, the pH of the pharmaceutical composition being from about 5.0 to about 5.4 (or 5.2±0.2, e.g., about 5.2) is indicative of suitability of the pharmaceutical composition for medical use. In some embodiments, pH is measured using a pH meter. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises particulate size and/or amount (e.g., particles per container) of the pharmaceutical composition (e.g., before and/or after storage). In some embodiments, the pharmaceutical composition is transferred into a vial, and the particles in the vial are examined using a particle sizing system (e.g., ACCUSIZER 780 SIS). In some embodiments, the particles with ≥10 μm diameter of ≤6000 per vial and particles with ≥25 μm diameter of ≤600 per vial is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises composition of the pharmaceutical composition (e.g., before and/or after storage). In some embodiments, the method comprises measuring one or more components of the pharmaceutical composition to assess the concentration and/or amount of the corresponding component. In some embodiments, the method comprises measuring the pH of the pharmaceutical composition, such as using a pH meter. Any suitable methods for measuring composition component(s) can be used herein, including but are not limited to, spectrophotometer, mass spectrometry (MS), rCE-SDS, nrCE-SDS, r-SDS-PAGE, nr-SDS-PAGE, SEC, HPLC (e.g., SE-HPLC, RP-HPLC), icIEF, or light-scattering detection (LSD). In some embodiments, the concentration of each component and/or the pH of the pharmaceutical composition within about 5% (e.g., within about any of 4%, 3%, 2%, 1%, 0.5%, 0.1%, or less) variation of that of each corresponding component and/or pH described herein is indicative of suitability of the pharmaceutical composition for medical use. In some embodiments, (a) the G-CSF dimer (e.g., G-CSF-Fc dimer, such as efbemalenograstim alfa) being about 1 mg/mL to about 100 mg/mL; (b) the buffering agent (e.g., sodium acetate) being about 1 mM to about 50 mM; (c) the stabilizing agent (e.g., EDTA) being about 0.1 mM to about 20 mM; (d) the tonicity agent (e.g., sorbitol) being about 1% (w/v) to about 15% (w/v) (e.g., about 1% (w/v) to about 10% (w/v)); (e) the surfactant (e.g., polysorbate 20) being about 0.001% (w/v) to about 0.1% (w/v); and (f) the pH of the pharmaceutical composition being about 4.2 to about 6.2 (e.g., about 5.0 to about 5.4), is indicative of suitability of the pharmaceutical composition for medical use. In some embodiments, (a) the G-CSF dimer (e.g., G-CSF-Fc dimer, such as efbemalenograstim alfa) being about 18 mg/mL to about 22 mg/mL (or 20.0±2.0 mg/mL, e.g., about 20 mg/mL); (b) the sodium acetate being about 10 mM; (c) the EDTA being about 1 mM; (d) the sorbitol being about 5% (w/v); (e) the polysorbate 20 being about 0.01% (w/v); and (f) the pH of the pharmaceutical composition being about 5.0 to about 5.4 (or 5.2±0.2, e.g., about 5.2), is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises potency of the pharmaceutical composition (e.g., before and/or after storage). In some embodiments, about 70% to about 130% (e.g., about any of 70%, 75%, 80%, 85%, 90%, 95%, 100%, 110%, 120%, or 130%) potency (e.g., potency of G-CSF), such as by cell proliferation assay, is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months. Any suitable methods for measuring G-CSF activity can be used herein, including but are not limited to, G-CSF receptor binding assay, reporter cell assay (e.g., using cells expressing G-CSF receptor, such as to measure tyrosine phosphorylation of JAK kinases or transcriptional factors), cell proliferation and/or differentiation assay (e.g., using cells expressing G-CSF receptor, such as neutrophilic granulocytes), granulocyte colony-forming assay, or in vivo assay (e.g., by measuring neutrophil count). The activity of G-CSF can be determined by means of conventional activity tests, as they are described in the art; see e.g., Draft Monographie “Filgrastim Concentrated Solution” Pharm Eur. Vol. 19, No. 1, January 2007, or Stute, N., et al. “Pharmacokinetics of subcutaneous recombinant human granulocyte colony-stimulating factor in children 1” (1992) Blood 79 (11), pages 2849-2854. The measurement of G-CSF activity in vitro is described, e.g., by Shirafuji, N. et al. 1989, A new bioassay for human granulocyte colony-stimulating factor (hG-CSF) using murine myeloblastic NFS-60 cells as targets and estimation of its levels in sera from normal healthy persons and patients with infectious and hematological disorders, Exp. Hematol. (1989) 17, 116-119. For the measurement of G-CSF activity in vivo see e.g., Tanaka, H. et al. 1991, Pharmacokinetics of recombinant human granulocyte colony-stimulating factor conjugated to polyethylene glycol in rats, Cancer Research (1991) 51, 3710-3714. Further publications where tests for the measurement of the activity of G-CSF are described are U.S. Pat. No. 6,555,660; Nohynek, G. J. et al. 1997, Comparison of the potency of glycosylated and nonglycosylated recombinant human granulocyte colony-stimulating factors in neutropenic and normeutropenic CD rats, Cancer Chemother. Pharmacol. (1997) 39, 259-266. Also see Example 8.

In some embodiments, the quality control parameter comprises amount and/or percentage of G-CSF dimer (e.g., before and/or after storage). In some embodiments, at least about 96% (such as at least about any of 97%, 98%, 99%, or 100%) of the G-CSF dimer in the pharmaceutical composition as measured by SEC is indicative of suitability of the pharmaceutical composition for medical use. In some embodiments, the pharmaceutical composition comprising at least about 95.5% (such as at least about any of 96%, 97%, 98%, 99%, or 100%) of the G-CSF dimer as measured by rCE-SDS is indicative of suitability of the pharmaceutical composition for medical use. In some embodiments, the pharmaceutical composition comprising at least about 94% (such as at least about any of 95%, 96%, 97%, 98%, 99%, or 100%) of the G-CSF dimer as measured by nrCE-SDS is indicative of suitability of the pharmaceutical composition for medical use. In some embodiments, the pharmaceutical composition comprising no more than about 1.5% (such as no more than about any of 1.4%, 1.3%, 1.2%, 1.1%, 1.0%, 0.8%. 0.5%, 0.3%, 0.1%, or less) of HMWS as measured by rCE-SDS is indicative of suitability of the pharmaceutical composition for medical use. In some embodiments, the pharmaceutical composition comprising no more than about 1.5% (such as no more than about any of 1.4%, 1.3%, 1.2%, 1.1%, 1.0%, 0.8%. 0.5%, 0.3%, 0.1%, or less) of HMWS as measured by nrCE-SDS is indicative of suitability of the pharmaceutical composition for medical use. In some embodiments, the pharmaceutical composition comprising about 27.2% to about 39.0% (e.g., about 30% to about 40%, such as about 32% to about 37%) acidic region, about 36.5% to about 60.1% (e.g., about 45% to about 55%, such as about 47% to about 55%) main peak, and about 7.2% to about 30% (e.g., about 10% to about 20%, such as about 11% to 18%) basic region as measured by icIEF is indicative of suitability of the pharmaceutical composition for medical use. In some embodiments, the concentration (e.g., G-CSF dimer concentration) of the pharmaceutical composition varying by less than about 0.5 mg/mL (e.g., less than about any of 0.4 mg/mL, 0.3 mg/mL, 0.2 mg/mL, 0.1 mg/mL, 0.05 mg/mL, or less) is indicative of suitability of the pharmaceutical composition for medical use. In some embodiments, the concentration (e.g., G-CSF dimer concentration) of the pharmaceutical composition being 20.0±2.0 mg/mL (e.g., by UV spec scan) is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises viscosity (e.g., before and/or after storage). In some embodiments, the viscosity of the pharmaceutical composition being from about 1 to about 50 mPa-S, such as any of about 1 to about 45 mPa-S, about 1 to about 40 mPa-S, about 1 to about 35 mPa-S, about 1 to about 30 mPa-S, about 1 to about 25 mPa-S, or about 1 to about 20 mPa-S, about 1 to about 15 mPa-S, about 1 to about 10 mPa-S, about 1 to about 5 mPa-S, about 1 to about 4 mPa-S, about 1 to about 3 mPa-S, or about 1 to about 20 mPa-S, at between about 5° C. to about 25° C., is indicative of suitability of the pharmaceutical composition for medical use. For example, in some embodiments, the viscosity of the pharmaceutical composition being about 1-2 mPa-S at about 25° C. is indicative of suitability of the pharmaceutical composition for medical use. In some embodiments, the pharmaceutical composition being isotonic is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises osmolality (e.g., before and/or after storage). In some embodiments, the osmolality of the pharmaceutical composition being from about 100 to about 500 mOsm/kg, such as any of about 120 to about 480 mOsm/kg, about 140 to about 440 mOsm/kg, about 160 to about 420 mOsm/kg, about 180 to about 400 mOsm/kg, about 200 to about 380 mOsm/kg, about 220 to about 360 mOsm/kg, about 270 to about 350 mOsm/kg, or about 268 to about 360 mOsm/kg is indicative of suitability of the pharmaceutical composition for medical use. In some embodiments, the osmolality of the pharmaceutical composition being about 310 mOsm/kg is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises protein aggregation degree (e.g., before and/or after storage). In some embodiments, the percentage of total protein aggregation being less than about 5%, such as less than about any of 4%, 3%, 2%, 1%, 0.5%, 0.2%, 0.1%, or less, is indicative of suitability of the pharmaceutical composition for medical use. In some embodiments, the percentage of total protein aggregation being less than about 2% (e.g., less than about any of 1.5%, 1%, 0.5%, 0.2%, 0.1%, or less) as measured by SEC is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises the percentage and/or amount of HMWS (e.g., before and/or after storage). In some embodiments, the pharmaceutical composition comprising no more than about 1.5% (e.g., no more than about any of 1.4%, 1.3%, 1.2%, 1.1, 1%, 0.8%, 0.5%, 0.1%, or less, such as no more than about 1%) HMWS as measured by rCE-SDS after at least about 1 month of storage under 25±2° C. is indicative of suitability of the pharmaceutical composition for medical use. In some embodiments, the pharmaceutical composition comprising no more than about 1.5% (e.g., no more than about any of 1.4%, 1.3%, 1.2%, 1.1, 1%, 0.8%, 0.5%, 0.1%, or less, such as no more than 1%) HMWS as measured by rCE-SDS after at least about 2 months of storage under 25±2° C. is indicative of suitability of the pharmaceutical composition for medical use. In some embodiments, the pharmaceutical composition comprising no more than about 1.5% (such as no more than 1%) HMWS as measured by rCE-SDS after up to about 3 months of storage under 25±2° C. is indicative of suitability of the pharmaceutical composition for medical use. In some embodiments, the pharmaceutical composition comprising no more than about 1.5% (e.g., no more than about any of 1.4%, 1.3%, 1.2%, 1.1, 1%, 0.8%, 0.5%, 0.1%, or less) HMWS as measured by rCE-SDS after at least about 36 months of storage under about 2° C. to about 8° C. is indicative of suitability of the pharmaceutical composition for medical use. In some embodiments, the pharmaceutical composition comprising no more than about 1.5% HMWS as measured by rCE-SDS after up to about 36 months of storage under about 2° C. to about 8° C. is indicative of suitability of the pharmaceutical composition for medical use. In some embodiments, the pharmaceutical composition comprising no more than about 1.5% (e.g., no more than about any of 1.4%, 1.3%, 1.2%, 1.1, 1%, 0.8%, 0.5%, 0.1%, or less) HMWS as measured by nrCE-SDS after at least about 1 month of storage under 25±2° C. is indicative of suitability of the pharmaceutical composition for medical use. In some embodiments, the pharmaceutical composition comprising no more than about 1.5% (e.g., no more than about any of 1.4%, 1.3%, 1.2%, 1.1, 1%, 0.8%, 0.5%, 0.1%, or less) HMWS as measured by nrCE-SDS after at least about 2 months of storage under 25±2° C. is indicative of suitability of the pharmaceutical composition for medical use. In some embodiments, the pharmaceutical composition comprising no more than about 1.5% HMWS as measured by nrCE-SDS after up to about 3 months of storage under 25±2° C. is indicative of suitability of the pharmaceutical composition for medical use. In some embodiments, the pharmaceutical composition comprising no more than about 1.5% (e.g., no more than about any of 1.4%, 1.3%, 1.2%, 1.1, 1%, 0.8%, 0.5%, 0.1%, or less) HMWS as measured by nrCE-SDS after at least about 36 months of storage under about 2° C. to about 8° C. is indicative suitability of the pharmaceutical composition for medical use. In some embodiments, the pharmaceutical composition comprising no more than about 1.5% HMWS as measured by nrCE-SDS after up to about 36 months of storage under about 2° C. to about 8° C. is indicative suitability of the pharmaceutical composition for medical use.

In some embodiments, the quality control parameter comprises the percentage and/or amount of low molecular weight species (LMWS) (e.g., before and/or after storage). In some embodiments, the pharmaceutical composition comprising no more than about 4% (e.g., no more than about any of 3.5%, 3%, 2.5%, 2%, 1.5%, 1%, 0.8%, 0.5%, 0.1%, or less) LMWS as measured by rCE-SDS is indicative of suitability of the pharmaceutical composition for medical use. In some embodiments, the pharmaceutical composition comprising no more than about 6% (e.g., no more than about any of 5.5%, 5%, 4.5%, 4%, 3.5%, 3%, 2.5%, 2%, 1.5%, 1%, 0.8%, 0.5%, 0.1%, or less) LMWS as measured by nrCE-SDS is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months.

In some embodiments, the quality control parameter comprises percentage and/or amount of the G-CSF dimer (e.g., before and/or after storage). In some embodiments, the pharmaceutical composition comprising at least about 90% (such as at least about any of 91%, 92%, 93%, 94%, 95%, 96%, 97%, 98%, 99%, or 100%) of the G-CSF dimer is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months. In some embodiments, the pharmaceutical composition comprising at least about 95.5% (e.g., at least about any of 96%, 97%, 98%, 99%, or 100%) of the G-CSF dimer as measured by rCE-SDS after at least about 1 month of storage under 25±2° C. is indicative of suitability of the pharmaceutical composition for medical use. In some embodiments, the pharmaceutical composition comprising at least about 95.5% (e.g., at least about any of 96%, 97%, 98%, 99%, or 100%) of the G-CSF dimer as measured by rCE-SDS after at least about 2 months of storage under 25±2° C. is indicative of suitability of the pharmaceutical composition for medical use. In some embodiments, the pharmaceutical composition comprising at least about 95.5% of the G-CSF dimer as measured by rCE-SDS after up to about 3 months of storage under 25±2° C. is indicative of suitability of the pharmaceutical composition for medical use. In some embodiments, the pharmaceutical composition comprising at least about 95.5% (e.g., at least about any of 96%, 97%, 98%, 99%, or 100%) of the G-CSF dimer as measured by rCE-SDS after at least about 36 months of storage under about 2° C. to about 8° C. is indicative of suitability of the pharmaceutical composition for medical use. In some embodiments, the pharmaceutical composition comprising at least about 95.5% of the G-CSF dimer as measured by rCE-SDS after up to about 36 months of storage under about 2° C. to about 8° C. is indicative of suitability of the pharmaceutical composition for medical use. In some embodiments, the pharmaceutical composition comprising at least about 94% (e.g., at least about any of 95%, 96%, 97%, 98%, 99%, or 100%) of the G-CSF dimer as measured by nrCE-SDS after at least about 1 month of storage under 25±2° C. is indicative of suitability of the pharmaceutical composition for medical use. In some embodiments, the pharmaceutical composition comprising at least about 94% (e.g., at least about any of 95%, 96%, 97%, 98%, 99%, or 100%) of the G-CSF dimer as measured by nrCE-SDS after at least about 2 months of storage under 25±2° C. is indicative of suitability of the pharmaceutical composition for medical use. In some embodiments, the pharmaceutical composition comprising at least about 94% of the G-CSF dimer as measured by nrCE-SDS after up to about 3 months of storage under 25±2° C. is indicative of suitability of the pharmaceutical composition for medical use. In some embodiments, the pharmaceutical composition comprising at least about 94% (e.g., at least about any of 95%, 96%, 97%, 98%, 99%, or 100%) of G-CSF dimer as measured by nrCE-SDS after at least about 36 months of storage under about 2° C. to about 8° C. is indicative of suitability of the pharmaceutical composition for medical use. In some embodiments, the pharmaceutical composition comprising at least about 94% of G-CSF dimer as measured by nrCE-SDS after up to about 36 months of storage under about 2° C. to about 8° C. is indicative of suitability of the pharmaceutical composition for medical use.

In some embodiments, the quality control parameter comprises percentage of the G-CSF dimer as measured by SEC (e.g., before and/or after storage). In some embodiments, the pharmaceutical composition comprising at least about 96% (e.g., at least about any of 97%, 98%, 99%, or 100%) of the G-CSF dimer as measured by SEC is indicative of suitability of the pharmaceutical composition for medical use. In some embodiments, the pharmaceutical composition comprising a main peak of at least about 96% (e.g., at least about any of 97%, 98%, 99%, or 100%) in relation to the total area of all peaks when measured by SE-HPLC is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months. In some embodiments the pharmaceutical composition comprising at least about 96% (e.g., at least about any of 97%, 98%, 99%, or 100%) of the G-CSF dimer as measured by SE-HPLC after at least about 1 month of storage under 25±2° C. is indicative of suitability of the pharmaceutical composition for medical use. In some embodiments the pharmaceutical composition comprising at least about 96% (e.g., at least about any of 97%, 98%, 99%, or 100%) of the G-CSF dimer as measured by SE-HPLC after at least about 2 months of storage under 25±2° C. is indicative of suitability of the pharmaceutical composition for medical use. In some embodiments the pharmaceutical composition comprising at least about 96% of the G-CSF dimer as measured by SE-HPLC after up to about 3 months of storage under 25±2° C. is indicative of suitability of the pharmaceutical composition for medical use. In some embodiments, the pharmaceutical composition comprising at least about 96% (e.g., at least about any of 97%, 98%, 99%, or 100%) of the G-CSF dimer as measured by SE-HPLC after at least about 36 months of storage under about 2° C. to about 8° C. is indicative of suitability of the pharmaceutical composition for medical use. In some embodiments, the pharmaceutical composition comprising at least about 96% of the G-CSF dimer as measured by SE-HPLC after up to about 36 months of storage under about 2° C. to about 8° C. is indicative of suitability of the pharmaceutical composition for medical use. In some embodiments, the pharmaceutical composition comprising a main peak at least about 85% (e.g., at least about any of 90%, 95%, 96%, 97%, 98%, 99%, or 100%) in relation to the total area of all peaks when measured by SE-HPLC after at least about 1 month of storage under 25±2° C. is indicative of suitability of the pharmaceutical composition for medical use. In some embodiments, the pharmaceutical composition comprising a main peak at least about 85% (e.g., at least about any of 90%, 95%, 96%, 97%, 98%, 99%, or 100%) in relation to the total area of all peaks when measured by SE-HPLC after at least about 2 months of storage under 25±2° C. is indicative of suitability of the pharmaceutical composition for medical use. In some embodiments, the pharmaceutical composition comprising a main peak at least about 85% in relation to the total area of all peaks when measured by SE-HPLC after up to about 3 months of storage under 25±2° C. is indicative of suitability of the pharmaceutical composition for medical use. In some embodiments, the pharmaceutical composition comprising a main peak at least about 85% (e.g., at least about any of 90%, 95%, 96%, 97%, 98%, 99%, or 100%) in relation to the total area of all peaks when measured by SE-HPLC after at least about 36 months of storage under about 2° C. to about 8° C. is indicative of suitability of the pharmaceutical composition for medical use. In some embodiments, the pharmaceutical composition comprising a main peak at least about 85% in relation to the total area of all peaks when measured by SE-HPLC after up to about 36 months of storage under about 2° C. to about 8° C. is indicative of suitability of the pharmaceutical composition for medical use.

In some embodiments, the quality control parameter comprises percentage of dimers measured by SEC (e.g., before and/or after storage). In some embodiments, the pharmaceutical composition comprising dimers less than about 5% (e.g., less than about any of 4%, 3%, 2%, 1.5%, 1%, 0.5%, 0.1%, or less, such as less than about 2.5%) in relation to the total area of all peaks when measured by SE-HPLC is indicative of suitability of the pharmaceutical composition for medical use. In some embodiments, the quality control parameter is measured before storage. In some embodiments, the quality control parameter is measured after storage. In some embodiments, the storage condition comprises storing at about 2° C. to about 8° C. up to about 36 months. In some embodiments, the storage condition comprises storing at 25±2° C. up to about 3 months. In some embodiments, the pharmaceutical composition comprising dimers less than about 5% (e.g., less than about any of 4%, 3%, 2%, 1.5%, 1%, 0.5%, 0.1%, or less, such as less than about 2.5%) in relation to the total area of all peaks when measured by SE-HPLC after at least about 1 month of storage under about 25±2° C. is indicative of suitability of the pharmaceutical composition for medical use. In some embodiments, the pharmaceutical composition comprising dimers less than about 5% (e.g., less than about any of 4%, 3%, 2%, 1.5%, 1%, 0.5%, 0.1%, or less, such as less than about 2.5%) in relation to the total area of all peaks when measured by SE-HPLC after at least about 2 months of storage under 25±2° C. is indicative of suitability of the pharmaceutical composition for medical use. In some embodiments, the pharmaceutical composition comprising dimers less than about 5% (e.g., less than about 2.5%) in relation to the total area of all peaks when measured by SE-HPLC after up to about 3 months of storage under 25±2° C. is indicative of suitability of the pharmaceutical composition for medical use. In some embodiments, the pharmaceutical composition comprising dimers less than about 5% (e.g., less than about any of 4%, 3%, 2%, 1.5%, 1%, 0.5%, 0.1%, or less, such as less than about 2.5%) in relation to the total area of all peaks when measured by SE-HPLC after at least about 36 months of storage under about 2° C. to about 8° C. is indicative of suitability of the pharmaceutical composition for medical use. In some embodiments, the pharmaceutical composition comprising dimers less than about 5% (e.g., less than about 2.5%) in relation to the total area of all peaks when measured by SE-HPLC after up to about 36 months of storage under about 2° C. to about 8° C. is indicative of suitability of the pharmaceutical composition for medical use.

In some embodiments, the quality control parameter comprises peaks as measured by icIEF (e.g., before and/or after storage). In some embodiments, the pharmaceutical composition (e.g., liquid pharmaceutical composition) comprising about 27.2% to about 39.0% (such as about 32% to about 37%) acidic region, about 36.5% to about 60.1% (such as about 47% to about 55%) main peak, and about 7.2% to about 30% (such as about 11% to about 18%) basic region as measured by icIEF following storage at 25±2° C. for at least about 1 month is indicative of suitability of the pharmaceutical composition for medical use. In some embodiments, the pharmaceutical composition (e.g., liquid pharmaceutical composition) comprising about 27.2% to about 39.0% (such as about 32% to about 37%) acidic region, about 36.5% to about 60.1% (such as about 47% to about 55%) main peak, and about 7.2% to about 30% (such as about 11% to about 18%) basic region as measured by icIEF following storage at 25±2° C. for at least about 2 months is indicative of suitability of the pharmaceutical composition for medical use. In some embodiments, the pharmaceutical composition (e.g., liquid pharmaceutical composition) comprising about 27.2% to about 39.0% acidic region, about 36.5% to about 60.1% main peak, and about 7.2% to about 30% basic region as measured by icIEF following storage at 25±2° C. for up to about 3 months is indicative of suitability of the pharmaceutical composition for medical use. In some embodiments, the liquid pharmaceutical composition comprising about 27.2% to about 39.0% (such as about 32% to about 37%) acidic region, about 36.5% to about 60.1% (such as about 47% to about 55%) main peak, and about 7.2% to about 30% (such as about 11% to about 18%) basic region as measured by icIEF following storage at about 2° C. to about 8° C. for at least about 36 months is indicative of suitability of the pharmaceutical composition for medical use. In some embodiments, the liquid pharmaceutical composition comprising about 27.2% to about 39.0% acidic region, about 36.5% to about 60.1% main peak, and about 7.2% to about 30% basic region as measured by icIEF following storage at about 2° C. to about 8° C. for up to about 36 months is indicative of suitability of the pharmaceutical composition for medical use.

In some embodiments, the method comprises assessing two or more quality control parameters described above, including for example at least any of 2, 3, 4, 5, 6, 7, 8, 9, 10, or more quality control parameters described above.

Also provided are methods of releasing a batch (e.g., a commercial batch) of a pharmaceutical composition described herein, the method comprising: assessing the suitability of the pharmaceutical composition for medical use in an individual (e.g., a human individual) using a sample of the commercial batch according to any of the methods described herein (such as by assessing if one or more quality control parameters described herein are within quality control parameter threshold(s)), and releasing the commercial batch if the sampled pharmaceutical composition is suitable for medical use.

Also provided are methods of processing a sample of any of the pharmaceutical compositions described herein to validate the sample as suitable for medical use in an individual (e.g., a human individual), the method comprising: obtaining the sample from a commercial batch; and assessing the suitability of the sampled pharmaceutical composition for medical use in an individual (e.g., a human individual) using a sample of the commercial batch according to any of the methods described herein, such as by assessing if one or more quality control parameters described herein are within quality control parameter threshold(s).

Also provided is a commercial batch of any of the pharmaceutical compositions described herein, wherein the commercial batch is optionally assessed, validated, released, or processed according to any of the methods described herein.

### IV. G-CSF Molecules

Human G-CSF is a glycoprotein having 204 amino acids with 30 amino-acid signal peptides. Mature G-CSF protein, having 18-20 kDa in molecular weight, is composed of 174 amino acids without signal peptides and secreted out of the cells. Human cells mainly responsible for such secretion are monocytes, fibroblasts, and endothelial cells. There are three main biological functions for G-CSF, namely: 1) acting on myeloid precursors and stem cells to drive the differentiation, development, and maturation of neutrophils; 2) activating mature neutrophils to participate in immune response; and 3) acting with other hematopoietic growth factors such as stem cell factor, Flt-3 ligand, and GM-CSF to mobilize hematopoietic stem cells.

G-CSF receptor (G-CSFR) is proven to exist in bone marrow hematopoietic stem cells Sca+Lin−Th1low, precursor cells CD34+, committed granulocyte precursor cells, and mature neutrophils. G-CSFR is a specific receptor having a high affinity to G-CSF and 812 amino acids. Tamada et al. obtained the crystalline structure of the G-CSF:G-CSFR complex and the stoichiometry of G-CSF:G-CSFR complex was shown as a 2:2 ratio by the 2.8 angstrom diffraction analysis (PNAS, 2008, Vol. 103: 3135-3140). In other words, in each complex, each G-CSF binds to one receptor chain to form a G-CSF-receptor complex when two G-CSF-receptor complexes are brought to close proximity, a 2:2 dimer is formed as a result of this interaction. Under this circumstance, the carboxyl terminal of the G-CSF receptor is then able to activate the downstream signal molecules JAKs (Janus tyrosine kinases, primarily JAK2). Consequently, JAK2 actives STAT3 to switch on the transcriptional genes which are critical for neutrophil differentiation and proliferation and activation.

In 2003, Schabitz W. R. et al. reported that recombinant human G-CSF (rhG-CSF) was shown to have a protective functionality on nerve cells from the study on the ischemic animal model (Storke, 2003, 34; 745-751). Later in 2006, Shyu et al. reported that rhG-CSF was shown to have clinical efficacy in the treatment of patients having acute stroke in which the patients were administrated with rhG-CSF daily for five consecutive days (CMAJ, 2006, 174:927-933). The in vivo half-life of rat G-CSF upon subcutaneous administration is about 2 hours, whereas the half-life of human G-CSF upon subcutaneous administration is only 3.5 hours. Therefore, it is required to administrate patients in need thereof with the drug daily, or intravenous infusion and this will affect the living quality of patients.

Pegfilgrastim, also known as Neulasta®, SD-01, and PEG-rmetHuG-CSF, is a G-CSF drug that has been developed to diminish the severity and duration of severe neutropenia, as well as complications of neutropenia, associated with the administration of myelosuppressive anti-cancer drugs or radiotherapy. Pegfilgrastim consists of a recombinant methionyl human G-CSF (r-metHuG-CSF) covalently attached to a monomethoxypolyethylene glycol (mPEG) via an amide bond. The r-metHuG-CSF is identical to the natural human G-CSF (SEQ ID NO: 1) except for the N-terminal methionine necessary for expression in E. coli. It is believed that the conjugation of mPEG to the N-terminal methionine of r-metHuG-CSF increases the serum half-life of r-metHuG-CSF (about 15-80 hours), reducing the frequency of administration required to maintain therapeutic neutrophil counts.

Eflapegrastim, also known as ROLONTIS®, SPI-2012, HM10460A, and ¹⁷⁻⁶⁵S-G-CSF, is a long-acting G-CSF drug that has been developed to reduce the severity and duration of severe neutropenia, as well as complications of neutropenia, associated with the use of myelosuppressive anti-cancer drugs or radiotherapy. Eflapegrastim consists of a recombinant human G-CSF analog (ef-G-CSF) and a recombinant fragment of the Fc region of human IgG4, linked by a bifunctional polyethylene glycol linker. ef-G-CSF varies from human G-CSF (SEQ ID NO: 1) at positions 17 and 65 which are substituted with serine. It is believed that the Fc region of human IgG4 increases the serum half-life of ef-G-CSF to about 36.4 hours (range: about 16.1 to about 115 hours).

Both pegfilgrastim and eflapegrastim have a G-CSF molecule in monomer form.

G-CSF-based drugs, such as filgrastim (Neupogen®) and its biosimilars Filgrastim-aafi (Nivestym®), Filgrastim-sndz (Zarxio®), Filgrastim-ayow (Releuko®), are used to manage neutropenia for patients receiving chemotherapy medications. However, these filgrastim or similar drugs require daily administration because of their short 3.5 hours half-life. Other G-CSF drugs, such as pegfilgrastim (e.g., Neulasta®) and eflapegrastim (e.g., ROLVEDON™), can be administered once per chemotherapy cycle because of the extended half-life of 30-50 hours through pegylated technology or fusion protein strategy. At present, the recommended dosing regimen for both eflapegrastim (Rolontis®, HM10460A) and pegfilgrastim is next-day administration following cytotoxic chemotherapy.

### V. G-CSF Dimer

In some embodiments, the G-CSF molecule is a G-CSF dimer. Any of the G-CSF dimers described herein can be used in the pharmaceutical compositions and methods of use described herein.

As used herein, the term “G-CSF dimer” refers to a protein comprising (or consisting essentially of) 2 units of G-CSF molecules, such as two units of any of the G-CSF monomers described herein, or 2 units of any of the monomeric subunits comprising any of the G-CSFs described herein. In a non-limiting example, a G-CSF dimer may comprise (or consist essentially of, or consist of) two G-CSF monomers directly connected to each other, or connected together via a linking moiety such as a peptide linker, a chemical bond, a covalent bond, or a polypeptide (e.g., carrier protein, dimerization domain). In another non-limiting example, a G-CSF dimer may comprise two monomeric subunits each comprising a G-CSF monomer connected to a carrier protein (e.g., albumin, or Fc domain). Further examples of the G-CSF dimers that may find use in the present inventions are described in U.S. Pat. Nos. 8,557,546, 9,642,917, and U.S. Patent Application US20130165637, the contents of each of which are incorporated herein by reference in their entirety.

As used herein, the term “G-CSF monomer” refers to one unit of a G-CSF protein or molecule. The terms “G-CSF,” “G-CSF molecule,” and “G-CSF protein” are used herein interchangeably.

The G-CSF monomer used in the G-CSF dimer can be derived from G-CSF molecule of any organism, such as human or non-human animals, including but not limited to a mammal such as primates (e.g., cynomolgus monkeys, rhesus monkeys), cattle, pigs, horses, sheep, goats, rodents, cats, and dogs. The G-CSF monomer can be a wildtype G-CSF, or a mutant G-CSF, such as a mutant G-CSF capable of producing most or full biological activity of a wild-type G-CSF. In some embodiments, the G-CSF monomer is a mature G-CSF. In some embodiments, the G-CSF monomer is a G-CSF functional fragment capable of producing most or full biological activity of a full length or mature G-CSF. In some embodiments, the G-CSF monomer is a murine G-CSF. In some embodiments, the G-CSF monomer is a human G-CSF (hG-CSF). In some embodiments, the G-CSF monomer comprises (or consists of) the amino acid sequence of SEQ ID NO: 1.

In some embodiments, the G-CSF dimer is a recombinant protein comprising two G-CSF (e.g., hG-CSF) molecules, such as produced in suitable host cells (e.g., CHO cells). In some embodiments, the G-CSF dimer comprises (or consists essentially of) two G-CSF (e.g., hG-CSF) monomers, such as two G-CSF monomers connected to each other via a linker (e.g., peptide linker). In some embodiments, the two G-CSF monomers forming the G-CSF dimer are the same (e.g., both comprising the sequence of SEQ ID NO: 1). In some embodiments, the two G-CSF monomers forming the G-CSF dimer are different.

In some embodiments, the G-CSF dimer comprises (or consists essentially of, or consists of) two G-CSF (e.g., hG-CSF) monomers and a carrier protein. In some embodiments, the carrier protein is an albumin (e.g., human albumin). In some embodiments, the carrier protein is an Fc domain of an immunoglobulin (e.g., human IgG1, IgG2, IgG3, or IgG4). In some embodiments, the two G-CSF monomers forming the G-CSF dimer are the same (e.g., both comprising the sequence of SEQ ID NO: 1). In some embodiments, the two G-CSF monomers forming the G-CSF dimer are different.

In some embodiments, the G-CSF dimer comprises Formula (I):

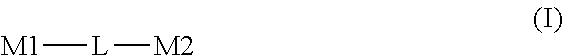

wherein M1 is a first monomer of G-CSF; M2 is a second monomer of G-CSF; and L is a linker connecting said first monomer and said second monomer and disposed there between. In some embodiments, the linker L is selected from the group consisting of: i) a short peptide comprising about 3 to about 50 amino acids; and ii) a polypeptide of formula (II):

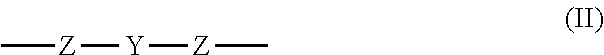

wherein Y is a carrier protein (e.g., albumin, Fc domain); Z is null, or a short peptide(s) comprising about 1 to about 30 amino acids; “-” is a chemical bond or a covalent bond. Any suitable linkers or short peptides that can provide flexibility between the 2 G-CSF monomers or between the G-CSF monomer and the carrier protein, and/or ensure the binding of each G-CSF monomer to its receptor can be used herein. In some embodiments, the linker L or the short peptide Z comprises the amino acid sequence of any of SEQ ID NOs: 12-31.

The carrier protein described herein can be any protein suitable for connecting two G-CSF monomers to form a G-CSF dimer, including but not limited to an Fc fragment of immunoglobulin (e.g., human IgG1, IgG2, IgG3, IgG4), or albumin (e.g., human serum albumin). When the carrier protein is formed by the connection of two protein subunits (e.g., via disulfide bond, peptide linkage, or chemical linkage), each protein subunit is referred to as a dimerization domain. In some embodiments, the carrier protein is formed by the connection of two dimerization domains (e.g., two Fc fragments of IgG) via one or more disulfide bonds. In some embodiments, the two dimerization domains forming the carrier protein are the same (e.g., two IgG2 Fc fragments). In some embodiments, the two dimerization domains forming the carrier protein are different. For example, in some embodiments, the carrier protein is formed by the connection of a first Fc fragment and a second different Fc fragment via one or more disulfide bonds.

In some embodiments, the G-CSF dimer comprises (or consists essentially of, or consists of) two monomeric subunits, wherein each monomeric subunit comprises (or consists essentially of, or consists of) a G-CSF monomer (e.g., hG-CSF monomer) and a dimerization domain. When the dimerization domain is an Fc fragment, such G-CSF dimer is also referred to as “G-CSF-Fc dimer.”

In some embodiments, the G-CSF monomer comprises the amino acid sequence of SEQ ID NO: 1, or a variant thereof having at least about 90% sequence identity (such as at least about any of 91%, 92%, 93%, 94%, 95%, 96%, 97%, 98%, 99%, or more) to the amino acid sequences of SEQ ID NO: 1. In some embodiments, the G-CSF monomer comprises the amino acid sequence of SEQ ID NO: 1.

In some embodiments, within each monomeric subunit, the G-CSF monomer is directly connected to the dimerization domain.

In some embodiments, within each monomeric subunit, the G-CSF monomer is connected to the dimerization domain via an optional linker. In some embodiments, the linker is about 6 to about 30 amino acids in length, such as any of about 6, 7, 8, 9, 10, 11, 12, 13, 14, 15, 16, 17, 18, 19, 20, 21, 22, 23, 24, 25, 26, 27, 28, 29, or 30 amino acids in length. In some embodiments, the linker is 16 amino acids in length. Any suitable linkers that can provide flexibility between the G-CSF monomer and the dimerization domain can be used herein. In some embodiments, the linker comprises the amino acid sequence of any of SEQ ID NOs: 12-31. In some embodiments, the linker comprises the amino acid sequence of SEQ ID NO: 12.

The linker situated between the two G-CSF monomers, or between one G-CSF monomer and the carrier protein or the dimerization domain, can be null (no linker), a peptide linker, or a non-peptide linker. In some embodiments, the first linker connecting the first G-CSF monomer and the carrier protein (or the first dimerization domain) and the second linker connecting the second G-CSF monomer and the carrier protein (or the second dimerization domain) are the same. In some embodiments, the first linker connecting the first G-CSF monomer and the carrier protein (or the first dimerization domain) and the second linker connecting the second G-CSF monomer and the carrier protein (or the second dimerization domain) are different. In general, a linker does not affect or significantly affect the proper fold and conformation formed by the configuration of the two G-CSF monomers.

The linkers can be peptide linkers of any length. In some embodiments, the peptide linker is from about 1 amino acid (aa) to about 10 aa long, from about 2 aa to about 15 aa long, from about 5 aa to about 8 aa long, from about 1 aa to about 20 aa long, from about 21 aa to about 30 aa long, from about 1 aa to about 30 aa long, from about 10 aa to about 30 aa long, from about 3 aa to about 50 aa long, or from about 6 aa to about 30 aa long. In some embodiments, the peptide linker is about any of 1, 2, 3, 4, 5, 6, 7, 8, 9, 10, 11, 12, 13, 14, 15, 16, 17, 18, 19, or 20 amino acids long. In some embodiments, the peptide linker is about any of 21, 22, 23, 24, 25, 26, 27, 28, 29, or 30 amino acids long. In some embodiments, the peptide linker is about any of 31, 32, 33, 34, 35, 36, 37, 38, 39, 40, 41, 42, 43, 44, 45, 46, 47, 48, 49, or 50 amino acids long. For example, in some embodiments, the linker is about 3 to about 50 amino acids in length. In some embodiments, the linker is about 6 to about 30 amino acids in length.

A peptide linker can have a naturally occurring sequence or a non-naturally occurring sequence. For example, a sequence derived from the hinge region of a heavy chain only antibody can be used as a linker. See, for example, WO1996/34103. In some embodiments, the peptide linker is a human IgG1, IgG2, IgG3, or IgG4 hinge. In some embodiments, the peptide linker comprises the amino acid sequence of SEQ ID NO: 13. In some embodiments, the linker is a flexible linker. Exemplary flexible linkers include, but are not limited to, glycine polymers (G)ₙ (SEQ ID NO: 18), glycine-serine polymers (including, for example, (GS)ₙ (SEQ ID NO: 19), (GSGGS)ₙ (SEQ ID NO: 20), or (GGGGS)ₙ (SEQ ID NO: 22), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). Exemplary flexible linkers include but are not limited to any of SEQ ID NOs: 12 and 14-31. In some embodiments, the linker comprises (or consists essentially of, or consists of) the sequence of SEQ ID NO: 12. In some embodiments, the linker (e.g., comprising SEQ ID NO: 12) further comprises a sequence of SEQ ID NO: 13. The ordinarily skilled artisan will recognize that design of a G-CSF dimer can include linkers that are all or partially flexible, such that the linker can include a flexible linker portion as well as one or more portions that confer less flexible structure to provide a desired G-CSF dimer structure and function.

In some embodiments, the linker between the G-CSF monomer and the carrier protein (e.g., dimerization domain), or between 2 G-CSF monomers, is a stable linker (not cleavable by protease, especially MMPs).

Other linker considerations include the effect on physical or pharmacokinetic properties of the resulting G-CSF dimer, such as solubility, lipophilicity, hydrophilicity, hydrophobicity, stability (more or less stable as well as planned degradation), rigidity, flexibility, immunogenicity, modulation of G-CSF/G-CSF receptor binding, the ability to be incorporated into a micelle or liposome, and the like.

In some embodiments, the dimerization domain (e.g., Fc fragment) within each monomeric subunit comprises at least two cysteines capable of forming intermolecular disulfide bonds. In some embodiments, there are about 2 to about 4 disulfide bonds between the two dimerization domains (e.g., Fc fragments). In some embodiments, the dimerization domain comprises leucine zippers. In some embodiments, the dimerization domain comprises at least a portion of an Fc fragment. In some embodiments, the Fc fragment comprises CH2 and CH3 domains. In some embodiments, the dimerization domain is derived from an Fc fragment of any of IgA, IgD, IgE, IgG, and IgM, and subtypes thereof. In some embodiments, the Fc fragment is derived from IgG1 Fc, IgG2 Fc, IgG4 Fc, or a fragment or a variant thereof. In some embodiments, the dimerization domain is derived from an Fc fragment of human IgG2. In some embodiments, the dimerization domain is derived from an Fc fragment of human IgG1. In some embodiments, the dimerization domain is derived from an Fc fragment of human IgG4. In some embodiments, the dimerization domain is a wild-type Fc fragment. In some embodiments, the dimerization domain comprises one or more mutations, such as a mutation in the Fc fragment to reduce or abolish effector functions, e.g., decreased antibody dependent cellular cytotoxicity (ADCC) or reduced binding to FcTR. In some embodiments, the Fc fragment comprises L234A and L235A (“LALA”) substitutions (EU numbering). In some embodiments, the dimerization domain is an IgG2 Fc fragment comprising a P331S substitution according to EU numbering. In some embodiments, the IgG2 Fc fragment comprises the sequence of SEQ ID NO: 2 or 3. In some embodiments, the dimerization domain is an IgG1 Fc fragment comprising L234A, L235A, and P331S substitutions according to EU numbering. In some embodiments, the IgG1 Fc fragment comprises the sequence of SEQ ID NO: 4. In some embodiments, the dimerization domain is an IgG4 Fc fragment comprising S228P, L234A, and L235A substitutions according to EU numbering. In some embodiments, the IgG4 Fc fragment comprises the sequence of SEQ ID NO: 5. In some embodiments, the dimerization domain comprises a full-length Fc fragment. In some embodiments, the dimerization domain comprises an N-terminus truncated Fc fragment, such as truncated Fc fragment with less N-terminal cysteines in order to reduce disulfide bond mispairing during dimerization. In some embodiments, the Fc fragment is truncated at the N-terminus, e.g., lacks the first 1, 2, 3, 4, 5, 6, 7, 8, 9, or 10 amino acids of a complete immunoglobulin Fc domain. In some embodiments, the dimerization domain is an IgG2 Fc fragment with N-terminal “ERKCC” sequence (SEQ ID NO: 32) removed.

In some embodiments, each of the monomeric subunit comprises an Fc fragment of human IgG2 or a variant thereof comprises up to 5, 4, 3, 2, or 1 amino acid substitutions. In some embodiments, each of the monomeric subunit comprises an Fc fragment of human IgG1 or a variant thereof comprises up to 5, 4, 3, 2, or 1 amino acid substitutions. In some embodiments, each of the monomeric subunit comprises an Fc fragment of human IgG4 or a variant thereof comprises up to 5, 4, 3, 2, or 1 amino acid substitutions. In some embodiments, each of the monomeric subunit comprises an Fc fragment having at least about 95% (e.g., at least about any of 96%, 97%, 98%, 99%, or more) sequence identity to SEQ ID NOs: 2-5. In some embodiments, the Fc fragment comprises (or consists essentially of, or consists of) the sequence of any of SEQ ID NOs: 2-5. In some embodiments, the Fc fragment comprises (or consists essentially of, or consists of) the sequence of SEQ ID NO: 2.

In some embodiments, the G-CSF dimer comprises two monomeric subunits, wherein: i) each monomeric subunit comprises a G-CSF monomer comprising the amino acid sequence of SEQ ID NO: 1, and an Fc fragment comprising the amino acid sequence of SEQ ID NO: 2; ii) each monomeric subunit comprises a G-CSF monomer comprising the amino acid sequence of SEQ ID NO: 1, and an Fc fragment comprising the amino acid sequence of SEQ ID NO: 3; iii) each monomeric subunit comprises a G-CSF monomer comprising the amino acid sequence of SEQ ID NO: 1, and an Fc fragment comprising the amino acid sequence of SEQ ID NO: 4; or iv) each monomeric subunit comprises a G-CSF monomer comprising the amino acid sequence of SEQ ID NO: 1, and an Fc fragment comprising the amino acid sequence of SEQ ID NO: 5. In some embodiments, the G-CSF dimer comprises two monomeric subunits, wherein each monomeric subunit comprises a G-CSF monomer comprising the amino acid sequence of SEQ ID NO: 1, and an Fc fragment comprising the amino acid sequence of SEQ ID NO: 2. In some embodiments, within each monomeric subunit, the G-CSF monomer is connected to the Fc fragment via a linker, such as a linker comprising the amino acid sequence of SEQ ID NO: 12.

In some embodiments, the G-CSF monomer is C-terminal to the dimerization domain within each monomeric subunit. In some embodiments, the G-CSF monomer is N-terminal to the dimerization domain within each monomeric subunit.

In some embodiments, each monomeric subunit comprises (or consists essentially of, or consists of) the amino acid sequence of SEQ ID NO: 6, or a variant thereof having at least about 90% (e.g., at least about any of 91%, 92%, 93%, 94%, 95%, 96%, 97%, 98%, 99%, or more) sequence identity to the amino acid sequence of SEQ ID NO: 6. In some embodiments, each monomeric subunit comprises (or consists essentially of, or consists of) the amino acid sequence of SEQ ID NO: 7, or a variant thereof having at least about 90% (e.g., at least about any of 91%, 92%, 93%, 94%, 95%, 96%, 97%, 98%, 99%, or more) sequence identity to the amino acid sequence of SEQ ID NO: 7. In some embodiments, each monomeric subunit comprises (or consists essentially of, or consists of) the amino acid sequence of SEQ ID NO: 8, or a variant thereof having at least about 90% (e.g., at least about any of 91%, 92%, 93%, 94%, 95%, 96%, 97%, 98%, 99%, or more) sequence identity to the amino acid sequence of SEQ ID NO: 8. In some embodiments, each monomeric subunit comprises (or consists essentially of, or consists of) the amino acid sequence of SEQ ID NO: 9, or a variant thereof having at least about 90% (e.g., at least about any of 91%, 92%, 93%, 94%, 95%, 96%, 97%, 98%, 99%, or more) sequence identity to the amino acid sequence of SEQ ID NO: 9. In some embodiments, each monomeric subunit comprises (or consists essentially of, or consists of) the amino acid sequence of SEQ ID NO: 10, or a variant thereof having at least about 90% (e.g., at least about any of 91%, 92%, 93%, 94%, 95%, 96%, 97%, 98%, 99%, or more) sequence identity to the amino acid sequence of SEQ ID NO: 10.

In some embodiments, each monomeric subunit comprises (or consists essentially of, or consists of) the amino acid sequence of any of SEQ ID NOs: 6-10. In some embodiments, each monomeric subunit comprises (or consists essentially of, or consists of) the amino acid sequence of SEQ ID NO: 6.

Hence in some embodiments, the G-CSF dimer comprises two monomeric subunits each comprising (or consisting essentially of, or consisting of) the amino acid sequence of SEQ ID NO: 6. This G-CSF dimer (or G-CSF-Fc dimer) is also herein referred to as “F-627” or “efbemalenograstim alfa.” In some embodiments, each monomeric subunit of the G-CSF dimer is encoded by a nucleic acid comprising (or consisting essentially of, or consisting of) the sequence of SEQ ID NO: 11.

In some embodiments, an amino acid sequence not affecting the biological activity of the G-CSF dimer can be added to the N-terminal or C-terminal of one or both monomeric subunits (e.g., G-CSF monomer, or G-CSF-Fc monomeric subunit) of the G-CSF dimer. In some embodiments, such appended amino acid sequence is beneficial to expression (e.g., signal peptide), purification (e.g., 6×His sequence, the cleavage site of Saccharomyces cerevisiae α-factor signal peptide (Glu-Lys-Arg)), or enhancement of biological activity of the G-CSF dimer.

In some embodiments, the G-CSF dimer is efbemalenograstim alfa. Efbemalenograstim alfa is a long-acting human G-CSF and human IgG2 Fc fragment fusion protein, existing as a dimer consisting of two G-CSF-Fc monomeric submits covalently linked through disulfide bonds formed between the Fc fragment of the molecule. Each G-CSF-Fc monomeric submit comprises (or consists essentially of, or consists of) the amino acid sequence of SEQ ID NO: 6. The hIgG2 Fc fragment has a P297S substitution when numbered from the N-terminus of the entire polypeptide chain of the G-CSF-Fc monomeric subunit, which corresponds to a P331S substitution located in the CH2 domain according to EU numbering. The purpose of the P331S substitution is to reduce the complement-dependent cytotoxicity function (CDC) mediated by the Fc region of human IgG2. Efbemalenograstim alfa is a glycosylated protein, produced by Chinese hamster ovary cells using serum-free medium. Each G-CSF-Fc monomeric subunit of efbemalenograstim alfa has one N-linked glycosylation site at N263 when numbered from the N-terminus of the entire polypeptide chain of the G-CSF-Fc monomeric subunit, which corresponds to N297 located in the CH2 domain of Fc region according to EU numbering. Each G-CSF-Fc monomeric subunit also has one O-linked glycosylation site at T133 in the human G-CSF monomer. Without wishing to be bound by theory, it is believed that the IgG2 Fc fragment prolongs the serum half-life of human G-CSF.

Through specific binding to the G-CSF receptor, efbemalenograstim alfa stimulates the survival, proliferation, differentiation and function of neutrophil precursors and mature neutrophils. Efbemalenograstim alfa is developed to decrease the incidence of infection, as manifested by febrile neutropenia, in patients with non-myeloid malignancies receiving myelosuppressive anti-cancer drugs associated with a clinically significant incidence of febrile neutropenia (efbemalenograstim alfa administration is after (e.g., 24 hours or 48 hours after) myelosuppressive anti-cancer drug treatment).

### a) Substitution, Insertion, Deletion, and Variants

In some embodiments, G-CSF molecules (e.g., G-CSF dimer) having one or more amino acid variations (e.g., substitutions) are provided. Conservative substitutions are shown in Table 1 under the heading of “Preferred substitutions.” More substantial changes are provided in Table 1 under the heading of “exemplary substitutions,” and as further described below in reference to amino acid side chain classes. Amino acid substitutions may be introduced into a G-CSF molecule (e.g., G-CSF dimer) (e.g., substitution within one or more of G-CSF monomer, linker, dimerization domain such as Fc fragment) and the products screened for a desired activity, e.g., retained/improved receptor binding, decreased immunogenicity, or improved ADCC or complement-dependent cytotoxicity (CDC).

TABLE 1
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

Alterations (e.g., substitutions) may be made, e.g., to improve G-CSF affinity.

In some embodiments, substitutions, insertions, and/or deletions may occur within the G-CSF molecule (e.g., G-CSF dimer) so long as such alterations do not substantially reduce the ability of the G-CSF molecule to bind the G-CSF receptor. For example, conservative alterations (e.g., conservative substitutions as provided herein) that do not substantially reduce binding affinity may be made.

A useful method for identification of residues or regions of a G-CSF or variants thereof that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the G-CSF with the receptor is affected. Further substitutions may be introduced at the amino acid locations demonstrating functional sensitivity to the initial substitutions. Alternatively, or additionally, a crystal structure of a receptor-ligand complex to identify contact points between the G-CSF and the receptor can be made. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include fusion to the N- or C-terminus of a G-CSF molecule to a polypeptide which increases the serum half-life of the G-CSF molecule.

### b) Glycosylation Variants

In some embodiments, each G-CSF-Fc monomeric subunit within the G-CSF dimer has one N-linked glycosylation site at N297 located in the CH2 domain according to EU numbering. In some embodiments, each G-CSF-Fc monomeric subunit within the G-CSF dimer has one O-linked glycosylation site at T133 in the hG-CSF monomer relative to SEQ ID NO: 1.

In some embodiments, the G-CSF molecule (G-CSF dimer) is altered to increase or decrease the extent to which the protein is glycosylated. Addition or deletion of glycosylation sites (e.g., to an Fc) may be conveniently accomplished by altering the amino acid sequence such that one or more glycosylation sites is created or removed.

Where the G-CSF dimer comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the Fc region may be made in order to create Fc variants with certain improved properties.

In some embodiments, the G-CSF-Fc dimer has a carbohydrate structure that lacks fucose attached (directly or indirectly) to an Fc region. For example, the amount of fucose in such G-CSF-Fc dimer may be from about 1% to about 80%, from about 1% to about 65%, from about 5% to about 65%, or from about 20% to about 40%. The amount of fucose is determined by calculating the average amount of fucose within the sugar chain at N297, relative to the sum of all glycostructures attached to N297 (e.g., complex, hybrid and high mannose structures) as measured by MALDI-TOF mass spectrometry, as described in WO 2008/077546, for example. N297 refers to the asparagine residue located at about position 297 in the Fc region (EU numbering of Fc region residues); however, N297 may also be located about ±3 amino acids upstream or downstream of position 297, i.e., between positions 294 and 300, due to minor sequence variations in the Fc domain. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” proteins include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated proteins include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94 (4):680-688 (2006); and WO2003/085107).

In some embodiments, the G-CSF molecule (e.g., G-CSF dimer, such as G-CSF-Fc dimer) has bisected oligosaccharides, e.g., in which a biantennary oligosaccharide attached to the Fc region is bisected by GlcNAc. Such variants may have reduced fucosylation and/or improved ADCC function.

### c) Fc Region Variants

In some embodiments, the Fc fragment of the G-CSF-Fc dimer has a reduced effector function as compared to corresponding wildtype Fc fragment, such as reducing at least about any of 30%, 40%, 50%, 60%, 70%, 80%, 85%, 90%, 95%, or more effector function as measured by the level of antibody-dependent cellular cytotoxicity (ADCC).

In some embodiments, one or more amino acid modifications may be introduced into the Fc fragment of the G-CSF-Fc dimer, thereby generating an Fc region variant. The Fc region variant may comprise a human Fc region sequence comprising an amino acid modification (e.g., a substitution) at one or more amino acid positions.

In some embodiments, the Fc fragment possesses some but not all effector functions, which make it a desirable candidate for applications in which the half-life of the G-CSF-Fc dimer in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the G-CSF-Fc dimer lacks FcγR binding (hence likely lacking ADCC activity), but retains FcRn binding ability. The primary cells for mediating ADCC, Natural Killer (NK) cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (See Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the G-CSF-Fc dimer is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12):1759-1769 (2006)).

Fc domain variants with reduced effector function include those with substitution of one or more of Fc region residues 238, 265, 269, 270, 297, 327, and 329 (U.S. Pat. No. 6,737,056). Such Fc mutants include Fc mutants with substitutions at two or more of amino acid positions 265, 269, 270, 297, and 327, including the so-called “DANA” Fc mutant with substitution of residues 265 and 297 to alanine (U.S. Pat. No. 7,332,581). In some embodiments, the Fc fragment comprises an N297A mutation. In some embodiments, the Fc fragment comprises an N297G mutation.

Certain Fc variants with improved or diminished binding to FcRs are described. See, e.g., U.S. Pat. No. 6,737,056; WO 2004/056312, and Shields et al., J. Biol. Chem. 9 (2): 6591-6604 (2001).

In some embodiments, alterations are made in the Fc region that result in altered (i.e., either improved or diminished) C1q binding and/or CDC, e.g., as described in U.S. Pat. No. 6,194,551, WO 99/51642, and Idusogie et al. J. Immunol. 164: 4178-4184 (2000).

In some embodiments, the G-CSF-Fc dimer comprises a variant Fc region comprising one or more amino acid substitutions which alters half-life and/or changes binding to the neonatal Fc receptor (FcRn). Fc domains with increased half-lives and improved binding to FcRn, which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those Fc domains with one or more substitutions therein have altered binding of the Fc region to FcRn, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some embodiments, it may be desirable to create cysteine engineered G-CSF dimer moieties, in which one or more residues of a G-CSF dimer are substituted with cysteine residues. In particular embodiments, the substituted residues occur at accessible sites of the G-CSF dimer. By substituting those residues with cysteine, reactive thiol groups are thereby positioned at accessible sites of the G-CSF dimer and may be used to conjugate the G-CSF dimer to other moieties. In some embodiments, any one or more of the following residues may be substituted with cysteine: A118 (EU numbering) of hinge, and S400 (EU numbering) of Fc region. Cysteine engineered G-CSF dimer may be generated as described, e.g., in U.S. Pat. No. 7,521,541. Engineered G-CSF dimer variants can provide for various intermolecular and intramolecular linkages.

In some embodiments, the C-terminal lysine (K447) of the Fc region may be cleaved by endogenous carboxypeptidase digestion in mammalian cell culture. The absence of C-terminal lysine does not affect the structure or the stability of the Fc region. See, Harris et al., Journal of Chromatography A, 705 (1995) 129-134. In some embodiments, the G-CSF dimer comprises a Fc region wherein the C-terminus lysine residue is cleaved. In some embodiments, there is provided a pharmaceutical composition comprising a mixture of G-CSF dimers comprising Fc regions, wherein at least some G-CSF dimers comprise Fc regions with the C-terminus lysine residue cleaved.

### VI. Methods of Treatment

Also provided herein are methods of treating or preventing a disease or condition or modulating an immune response in an individual (e.g., human) in need thereof. The methods comprise administering to the individual an effective amount of any of the G-CSF dimer pharmaceutical compositions described herein (e.g., G-CSF-Fc dimer pharmaceutical composition, such as efbemalenograstim alfa pharmaceutical composition). In some embodiments, the disease or condition is selected from the group consisting of neutropenia, stroke, spinal injury, neurological disorders accompanied with blood brain barrier injury, Parkinson's disease, Alzheimer's disease, Huntington disease, amyotrophic lateral sclerosis, spinal muscular atrophy, primary lateral sclerosis, spinal cerebellar ataxias, and mobile of hematopoietic stem cells into peripheral blood in allogeneic blood stem cell transplantation. In some embodiments, the disease or condition is characterized by compromised white blood cell production. In some embodiments, the disease or condition is neutropenia, such as chemotherapy-induced neutropenia or radiotherapy-induced neutropenia. In some embodiments, the disease or condition is mobilization of hematopoietic stem cells into peripheral blood in allogeneic blood stem cell transplantation. In some embodiments, the pharmaceutical composition is administered at a dosage of from about 0.01 mg/kg to about 1 mg/kg. In some embodiments, the pharmaceutical composition is administered subcutaneously. In some embodiments, the pharmaceutical composition is administered once every three weeks.

In some embodiments, there is provided a method of treating or preventing a disease or condition (e.g., a condition characterized by compromised white blood cell production) in an individual (e.g., human, such as a human with cancer), comprising administering to the individual an effective amount of a pharmaceutical composition, wherein the pharmaceutical composition comprises (or consists essentially of, or consists of): (a) about 1 mg/mL to about 100 mg/mL (e.g., about 5 mg/mL to about 50 mg/mL, or about 20 mg/mL) of a G-CSF dimer (e.g., G-CSF-Fc dimer, such as efbemalenograstim alfa); (b) about 1 mM to about 50 mM (e.g., about 1 mM to about 30 mM, or about 10 mM) buffering agent (e.g., sodium acetate); (c) about 0.1 mM to about 20 mM (e.g., 0.1 mM to about 10 mM, or about 1 mM) stabilizing agent (e.g., EDTA); (d) about 1% (w/v) to about 15% (w/v) (e.g., about 1% (w/v) to about 10% (w/v), about 5% (w/v), or about 9% (w/v)) tonicity agent (e.g., sorbitol, or sucrose); and (e) about 0.001% (w/v) to about 0.1% (w/v) (e.g., about 0.005% (w/v) to about 0.05% (w/v), or about 0.01% (w/v)) surfactant (e.g., polysorbate 20, or polysorbate 80); wherein the pH of the pharmaceutical composition is about 4.2 to about 6.2 (e.g., about 4.8 to about 5.8, about 5.0 to about 5.4, or about 5.2). In some embodiments, there is provided a method of treating or preventing a disease or condition (e.g., a condition characterized by compromised white blood cell production) in an individual (e.g., human, such as a human with cancer), comprising administering to the individual an effective amount of a pharmaceutical composition, wherein the pharmaceutical composition comprises (or consists essentially of, or consists of): (a) about 18 mg/mL to about 22 mg/mL (e.g., about 20 mg/mL) of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.0 to about 5.4 (e.g., about 5.2). In some embodiments, there is provided a method of treating or preventing a disease or condition (e.g., a condition characterized by compromised white blood cell production) in an individual (e.g., human, such as a human with cancer), comprising administering to the individual an effective amount of a pharmaceutical composition, wherein the pharmaceutical composition comprises (or consists essentially of, or consists of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.2. In some embodiments, the G-CSF dimer comprises two monomeric subunits each comprising an amino acid sequence of SEQ ID NO: 1. In some embodiments, the G-CSF dimer comprises two monomeric subunits, wherein each monomeric subunit comprises a G-CSF monomer (e.g., SEQ ID NO: 1), an Fc fragment (e.g., any of SEQ ID NOs: 2-5), and an optional linker (e.g., SEQ ID NO: 12) connecting the G-CSF monomer and the Fc fragment. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is contained in a syringe (e.g., sterile and/or for single use). In some embodiments, the volume of the pharmaceutical composition within the syringe is about 1 mL. In some embodiments, the pharmaceutical composition is administered subcutaneously. In some embodiments, the disease or condition is neutropenia, such as chemotherapy-induced neutropenia or radiotherapy-induced neutropenia. In some embodiments, the method comprises administering to the individual the effective amount of the pharmaceutical composition after (e.g., 24 hours after, or 48 hours after) administration of a chemotherapeutic agent to the individual. In some embodiments, the chemotherapeutic agent is a myelosuppressive chemotherapeutic agent. In some embodiments, the pharmaceutical composition is administered once every three weeks. In some embodiments, the individual is administered at least 4 cycles (e.g., about 21 days per cycle) of the chemotherapeutic agent, and wherein the pharmaceutical composition is administered after (e.g., 24 hours after, or 48 hours after) the administration of the chemotherapeutic agent in each cycle of the at least 4 cycles. In some embodiments, the pharmaceutical composition is administered at about 5 mg to about 25 mg (e.g., about 20 mg) of the G-CSF dimer for each administration (e.g., once per chemotherapy cycle).

The methods described herein can be used to treat and/or prevent one or more conditions induced by or accompanying chemotherapy, including but not limited to: reduced (e.g., reducing at least about any of 5%, 10%, 20%, 50%, 60%, 70%, 80%, 90%, or more) absolute neutrophil count (ANC), reduced number of granulocytes, reduced stem cell production, reduced hematopoiesis, reduced number of hematopoietic progenitor cells (HPCs), and condition characterized by compromised white blood cell production.

In some embodiments, the disease or condition is characterized by compromised white blood cell production. In some embodiments, the condition characterized by compromised white blood cell production is neutropenia. In some embodiments, the condition characterized by compromised white blood cell production is chemotherapy-induced neutropenia or radiotherapy-induced neutropenia. In some embodiments, the neutropenia is drug-induced neutropenia. In some embodiments, the neutropenia is anti-cancer drug-induced neutropenia. In some embodiments, the anti-cancer drug is myelosuppressive. In some embodiments, the neutropenia is induced by myelosuppressive anti-cancer drug.

In some embodiments, there is provided a method of treating or preventing a condition characterized by compromised white blood cell production (e.g., chemotherapy-induced neutropenia, or infection) in an individual (e.g., human, such as a human with cancer), comprising administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of a chemotherapeutic agent to the individual. In some embodiments, there is provided a method of treating or preventing a condition characterized by compromised white blood cell production (e.g., chemotherapy-induced neutropenia, or infection) in an individual (e.g., human, such as a human with cancer), comprising administering to the individual an effective amount of a pharmaceutical composition after (e.g., at least 24 hours after) administration of a chemotherapeutic agent to the individual, wherein the pharmaceutical composition comprises (or consists essentially of, or consists of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.2. In some embodiments, there is provided a method of increasing (e.g., increasing at least about any of 10%, 20%, 50%, 70%, 90%, 1-fold, 2-fold, 5-told, 20-fold, or more) one or more of: i) the ANC, ii) the number of granulocytes (e.g., in a subject eligible for a bone marrow transplant), iii) stem cell production, iv) hematopoiesis, and v) the number of HPCs, in an individual (e.g., human, such as an individual having cancer) in need thereof, the method comprising administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of a chemotherapeutic agent to the individual. In some embodiments, there is provided a method of reducing (e.g., reducing at least about any of 5%, 10%, 20%, 30%, 40%, 50%, 60%, 70%, 80%, 90%, 95%, or more) the duration of chemotherapy-induced neutropenia (e.g., Grade 4) in an individual (e.g., human, such as an individual having cancer) in need thereof, the method comprising administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of a chemotherapeutic agent to the individual. In some embodiments, there is provided a method of reducing (e.g., reducing at least about any of 5%, 10%, 20%, 30%, 40%, 50%, 60%, 70%, 80%, 90%, 95%, or more) or preventing the incidence of chemotherapy-induced neutropenia (e.g., Grade 4) in an individual (e.g., human, such as an individual having cancer) in need thereof, the method comprising administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of a chemotherapeutic agent to the individual. In some embodiments, the method reduces the duration of an ANC of less than about 0.5×10⁹/L in the individual to less than about 36 hours (e.g., less than about any of 34, 32, 30, 28, 26, 24, 23, 22, 21, 20, 19, 18, 17, 16, 15, 14, 13, 12, 11, 10, 9, 8, 7, 6, 5.5, 5, 4.5, 4, 3.5, 3, 2.5, 2, 1.5, 1, 0.5, 0.2, 0.1 hours, or less). In some embodiments, the method prevents the ANC in the individual from reaching less than about 0.5×10⁹/L (e.g., less than about any of 0.4×10⁹/L, 0.2×10⁹/L, 0.1×10⁹/L, 1.0×10⁷/L, or less). In some embodiments, the administration of the G-CSF dimer pharmaceutical composition increases the ANC from the first occurrence of less than about 0.5×10⁹/L to greater than or equal to about 2.0×10⁹/L within about 10 days (e.g., within about any of 9, 8, 7, 6, 5, 4, 3, 2, 1, 0.5, 0.2, 0.1 days, or less). In some embodiments, the administration of the G-CSF dimer increases the ANC from the first occurrence of less than about 0.5×10⁹/L to greater than or equal to about 2.0×10⁹/L within about 10 days (e.g., within about any of 9, 8, 7, 6, 5, 4, 3, 2, 1, 0.5, 0.2, 0.1 days, or less). In some embodiments, there is provided a method of reducing (e.g., reducing at least about any of 5%, 10%, 20%, 30%, 40%, 50%, 60%, 70%, 80%, 90%, 95%, or more) or preventing the incidence of febrile neutropenia (FN) in an individual (e.g., human, such as an individual having cancer) in need thereof, the method comprising administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of a chemotherapeutic agent to the individual. In some embodiments, there is provided a method of activating one or more of: i) neutrophil precursor cells, ii) myeloid stem cells, and iii) mature neutrophils, in an individual (e.g., human, such as an individual having cancer) in need thereof, the method comprising administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of a chemotherapeutic agent to the individual. In some embodiments, the pharmaceutical composition comprises (or consists essentially of, or consists of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.2. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the method comprises administering to the individual the effective amount of the pharmaceutical composition at least 24 hours after administration of the chemotherapeutic agent to the individual. In some embodiments, the chemotherapeutic agent is a myelosuppressive chemotherapeutic agent. In some embodiments, the myelosuppressive chemotherapeutic agent is selected from the group consisting of epirubicin, docetaxel, cyclophosphamide, doxorubicin, etoposide, cisplatin, paclitaxel, topotecan, vincristine, methylprednisolone, cytarabine, and a combination thereof. In some embodiments, the individual is administered two or more chemotherapeutic agents comprising: i) epirubicin and cyclophosphamide; ii) docetaxel and cyclophosphamide; iii) doxorubicin and cyclophosphamide; iv) docetaxel and doxorubicin; or v) docetaxel, doxorubicin, and cyclophosphamide. In some embodiments, the individual is administered the chemotherapeutic agent to treat cancer. In some embodiments, the cancer is selected from the group consisting of breast cancer, non-small cell lung cancer, small cell lung cancer, ovarian cancer, sarcoma, urothelial cancer, germ cell tumor, and non-Hodgkin's lymphoma. In some embodiments, the individual is administered at least 4 cycles of the chemotherapeutic agent, and wherein the pharmaceutical composition is administered after (e.g., at least 24 hours after) administration of the chemotherapeutic agent in each cycle of the at least 4 cycles. In some embodiments, the individual has breast cancer, (i) wherein the individual is administered at least 4 cycles of docetaxel and cyclophosphamide, wherein docetaxel 75 mg/m² and cyclophosphamide 600 mg/m² is administered on Day 1 of each cycle, and wherein the pharmaceutical composition is administered on Day 2 of each cycle (e.g., at least 24 hours after administration of docetaxel and cyclophosphamide); (ii) wherein the individual is administered at least 4 cycles of epirubicin and cyclophosphamide, wherein epirubicin 100 mg/m² and cyclophosphamide 600 mg/m² is administered on Day 1 of each cycle, and wherein the pharmaceutical composition is administered on Day 3 of each cycle (e.g., at least 48 hours after administration of epirubicin and cyclophosphamide); or (iii) wherein the individual is administered at least 4 cycles of docetaxel and doxorubicin, wherein docetaxel 75 mg/m² and doxorubicin 60 mg/m² is administered on Day 1 of each cycle, and wherein the pharmaceutical composition is administered on Day 2 of each cycle (e.g., at least 24 hours after administration of docetaxel and doxorubicin). In some embodiments, each cycle is about 21 days. In some embodiments, the pharmaceutical composition is administered at about 5 mg to about 25 mg (e.g., about 10 mg to about 25 mg, such as about 20 mg) of the G-CSF dimer for each administration. In some embodiments, the pharmaceutical composition is administered subcutaneously. In some embodiments, the pharmaceutical composition is contained (e.g., pre-filled) in a syringe (e.g., sterile and/or for single use). In some embodiments, the volume of the pharmaceutical composition within the syringe is about 1 mL.

In some embodiments, there is provided a method for treating cancer (e.g., breast cancer) in an individual (e.g., human), the method comprising: administering to the individual an effective amount of a chemotherapeutic agent, and further administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent to the individual, wherein the administration of the pharmaceutical composition alleviates or prevents a condition characterized by compromised white blood cell production (e.g., chemotherapy-induced neutropenia, or infection). In some embodiments, the condition characterized by compromised white blood cell production is chemotherapy-induced neutropenia. In some embodiments, the chemotherapeutic agent is a myelosuppressive chemotherapeutic agent. In some embodiments, the myelosuppressive chemotherapeutic agent is selected from the group consisting of epirubicin, docetaxel, cyclophosphamide, doxorubicin, etoposide, cisplatin, paclitaxel, topotecan, vincristine, methylprednisolone, cytarabine, and a combination thereof. In some embodiments, the individual is administered two or more chemotherapeutic agents comprising: i) epirubicin and cyclophosphamide; ii) docetaxel and cyclophosphamide; iii) doxorubicin and cyclophosphamide; iv) docetaxel and doxorubicin; or v) docetaxel, doxorubicin, and cyclophosphamide. In some embodiments, the two or more chemotherapeutic agents are administered simultaneously (e.g., in the same formulation, or in separate formulations). In some embodiments, the two or more chemotherapeutic agents are administered sequentially. In some embodiments, the cancer is selected from the group consisting of breast cancer, non-small cell lung cancer, small cell lung cancer, ovarian cancer, sarcoma, urothelial cancer, germ cell tumor, and non-Hodgkin's lymphoma. In some embodiments, the administration of the G-CSF dimer pharmaceutical composition increases (e.g., increasing at least about any of 10%, 20%, 50%, 70%, 90%, 1-fold, 2-fold, 5-told, 20-fold, or more) one or more of: i) the ANC, ii) the number of granulocytes (e.g., in a subject eligible for a bone marrow transplant), iii) stem cell production, iv) hematopoiesis, and v) the number of HPCs in the individual. In some embodiments, the administration of the G-CSF dimer pharmaceutical composition reduces (e.g., reducing at least about any of 5%, 10%, 20%, 30%, 40%, 50%, 60%, 70%, 80%, 90%, 95%, or more) the duration of chemotherapy-induced neutropenia (e.g., Grade 4) in the individual. In some embodiments, the administration of the G-CSF dimer pharmaceutical composition reduces (e.g., reducing at least about any of 5%, 10%, 20%, 30%, 40%, 50%, 60%, 70%, 80%, 90%, 95%, or more) or prevents the incidence of chemotherapy-induced neutropenia (e.g., Grade 4) in the individual. In some embodiments, the administration of the G-CSF dimer pharmaceutical composition reduces the duration of an ANC of less than about 0.5×10⁹/L in the individual to less than about 36 hours (e.g., less than about any of 34, 32, 30, 28, 26, 24, 23, 22, 21, 20, 19, 18, 17, 16, 15, 14, 13, 12, 11, 10, 9, 8, 7, 6, 5.5, 5, 4.5, 4, 3.5, 3, 2.5, 2, 1.5, 1, 0.5, 0.2, 0.1 hours, or less). In some embodiments, the administration of the G-CSF dimer pharmaceutical composition prevents the ANC in the individual from reaching less than about 0.5×10⁹/L (e.g., less than about any of 0.4×10⁹/L, 0.2×10⁹/L, 0.1×10⁹/L, 1.0×10⁷/L, or less). In some embodiments, the administration of the G-CSF dimer pharmaceutical composition increases the ANC from the first occurrence of less than about 0.5×10⁹/L to greater than or equal to about 2.0×10⁹/L within about 10 days (e.g., within about any of 9, 8, 7, 6, 5, 4, 3, 2, 1, 0.5, 0.2, 0.1 days, or less). In some embodiments, the administration of the G-CSF dimer pharmaceutical composition reduces (e.g., reducing at least about any of 5%, 10%, 20%, 30%, 40%, 50%, 60%, 70%, 80%, 90%, 95%, or more) or prevents the incidence of FN in the individual. In some embodiments, the administration of the G-CSF dimer pharmaceutical composition activates one or more of: i) neutrophil precursor cells, ii) myeloid stem cells, and iii) mature neutrophils in the individual. In some embodiments, the method of treating cancer can achieve one or more of the following biological activities: (1) killing (e.g., at least about any of 10%, 20%, 30%, 40%, 50%, 60%, 70%, 80%, 90%, or 100%) cancer cells; (2) inhibiting (e.g., at least about any of 10%, 20%, 30%, 40%, 50%, 60%, 70%, 80%, 90%, or 100%) proliferation of cancer cells; (3) reducing (e.g., at least about any of 10%, 20%, 30%, 40%, 50%, 60%, 70%, 80%, 90%, or 100%) tumor size; (4) alleviating (e.g., at least about any of 10%, 20%, 30%, 40%, 50%, 60%, 70%, 80%, 90%, or 100%) one or more symptoms in the individual having cancer; (5) inhibiting (e.g., at least about any of 10%, 20%, 30%, 40%, 50%, 60%, 70%, 80%, 90%, or 100%) tumor metastasis (e.g., metastasis to lymph nodes); (6) reducing (e.g., at least about any of 10%, 20%, 30%, 40%, 50%, 60%, 70%, 80%, 90%, or 100%) the presentation, incidence, or burden of pre-existing tumor metastasis (e.g., metastasis to lymph nodes); (7) prolonging survival, such as prolonging the survival of the individual by at least about any of 1, 2, 3, 4, 5, 6, 7, 8, 9, 10, 11, 12, 18, 24, or 48 months, or more; (8) prolonging the time to cancer progression, such as by at least about any of 1, 2, 3, 4, 5, 6, 7, 8, 9, 10, 11, 12, or 60 weeks, or more; and (9) preventing, inhibiting, or reducing (e.g., at least about any of 10%, 20%, 30%, 40%, 50%, 60%, 70%, 80%, 90%, or 100%) the likelihood of the recurrence of a cancer. In some embodiments, the pharmaceutical composition comprises (or consists essentially of, or consists of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.2. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is administered at about 5 mg to about 25 mg (e.g., about 10 mg to about 25 mg, such as about 20 mg) of the G-CSF dimer for each administration. In some embodiments, the pharmaceutical composition is administered subcutaneously. In some embodiments, the pharmaceutical composition is contained (e.g., pre-filled) in a syringe (e.g., sterile and/or for single use). In some embodiments, the volume of the pharmaceutical composition within the syringe is about 1 mL.

The G-CSF dimer (e.g., G-CSF-Fc dimer, such as efbemalenograstim alfa) pharmaceutical composition can be administered after administration of a chemotherapeutic agent to the individual, such as at least 24 hours after administration of the chemotherapeutic agent. In some embodiments, the pharmaceutical composition is administered 24, 28, 30, 32, 36, 40, 44, 48, 52, 60, 72, or more hours after administration of the chemotherapeutic agent, such as 24 hours after or 48 hours after administration of the chemotherapeutic agent.

In some embodiments, when the individual is administered 2 or more (e.g., 4) cycles of chemotherapeutic agent, the administration of the G-CSF dimer pharmaceutical composition is after (e.g., at least 24 hours after) administration of the last dose of the chemotherapeutic agent in the cycle. For example, if the chemotherapeutic agent is administered for one dose on Day 1 of a cycle, then the G-CSF dimer pharmaceutical composition is after (e.g., at least 24 hours after) administration of the one dose of the chemotherapeutic agent in the cycle. If the chemotherapeutic agent is administered for a first dose on Day 1 and a second dose on Day 2 of a cycle (i.e., a cycle of at least 2 days), then the G-CSF dimer pharmaceutical composition is after (e.g., at least 24 hours after) administration of the second dose of the chemotherapeutic agent in the cycle. Similarly, if the individual is administered 2 or more (e.g., 4) doses of chemotherapeutic agent for only one cycle, the administration of the G-CSF dimer pharmaceutical composition is after (e.g., at least 24 hours after) administration of the last dose of the chemotherapeutic agent. In some embodiments, when the individual is administered 2 or more (e.g., 4) cycles of chemotherapeutic agent, the administration of the G-CSF dimer pharmaceutical composition after (e.g., at least 24 hours after) administration of the chemotherapeutic agent is at the same time for each cycle, e.g., all at 24 hours after or at 48 hours after the administration of the chemotherapeutic agent for each cycle. In some embodiments, when the individual is administered 2 or more (e.g., 4) cycles of chemotherapeutic agent, the administration of the G-CSF dimer pharmaceutical composition after (e.g., at least 24 hours after) administration of the chemotherapeutic agent can be at different time for 2 or more cycles, e.g., at 24 hours after the administration of the chemotherapeutic agent in one cycle and at 48 hour after the administration of the chemotherapeutic agent in another cycle. In some embodiments, when the individual is administered 2 or more (e.g., 4) cycles of chemotherapeutic agent, the administration of the G-CSF dimer pharmaceutical composition is after (e.g., at least 24 hours after) administration of the chemotherapeutic agent at least in Cycle 1 (e.g., Cycle 1, Cycles 1-4, or all cycles).

In some embodiments, the individual is administered two or more chemotherapeutic agents (e.g., docetaxel and cyclophosphamide), such as two or more chemotherapeutic agents per cycle. In some embodiments, the two or more chemotherapeutic agents are administered simultaneously, either in a same formulation, or in separate formulations. In some embodiments, the two or more chemotherapeutic agents are administered sequentially, for example, the first chemotherapeutic agent is administered on Day 1 and the second chemotherapeutic agent is administered on Day 2. When the individual is administered two or more chemotherapeutic agents sequentially, in some embodiments, the administration of the G-CSF dimer pharmaceutical composition is after (e.g., at least 24 hours after) administration of the last chemotherapeutic agent, e.g., the last chemotherapeutic agent of the cycle. In some embodiments, when the two or more chemotherapeutic agents are administered at least 2 days apart, e.g., the first chemotherapeutic agent is administered on Day 1 and the second chemotherapeutic agent is administered on Day 3 or later, depending on the ANC level and/or other conditions (e.g., infection, fever, vital sign, laboratory measurements) of the individual to be treated, the administration of the G-CSF dimer pharmaceutical composition can be after (e.g., at least 24 hours after) administration of the first chemotherapeutic agent, or the G-CSF dimer pharmaceutical composition can be administered after (e.g., at least 24 hours after) administration of each of the two or more chemotherapeutic agents.

In some embodiments, the individual is administered at least 2 (e.g., 2, 3, or 4) cycles of the chemotherapeutic agent. In some embodiments, the G-CSF dimer pharmaceutical composition is administered after (e.g., at least 24 hours after) administration of the chemotherapeutic agent in Cycle 1. In some embodiments, the individual is administered at least 4 cycles of the chemotherapeutic agent, wherein the G-CSF dimer pharmaceutical composition is administered after (e.g., at least 24 hours after) administration of the chemotherapeutic agent in Cycle 1. Hence in some embodiments, there is provided a method for treating or preventing a condition characterized by compromised white blood cell production (e.g., chemotherapy-induced neutropenia, or infection) in an individual (e.g., human, such as an individual having cancer) in need thereof, wherein the individual is administered at least 2 (e.g., 2, 3, or 4) cycles of a chemotherapeutic agent (e.g., the chemotherapeutic agent is administered on Day 1 of each cycle), wherein the method comprises administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent in Cycle 1. In some embodiments, there is provided a method for treating or preventing a condition characterized by compromised white blood cell production (e.g., chemotherapy-induced neutropenia, or infection) in an individual (e.g., human, such as an individual having cancer) in need thereof, wherein the individual is administered at least 4 cycles (e.g., 4 cycles) of a chemotherapeutic agent (e.g., the chemotherapeutic agent is administered on Day 1 of each cycle), wherein the method comprises administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent in Cycle 1. In some embodiments, the individual is administered the chemotherapeutic agent to treat cancer (e.g., breast cancer). In some embodiments, there is provided a method for treating cancer (e.g., breast cancer) in an individual (e.g., human), the method comprising: i) administering to the individual an effective amount of a chemotherapeutic agent for at least 2 (e.g., 2, 3, or 4) cycles (e.g., the chemotherapeutic agent is administered on Day 1 of each cycle); and ii) administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent in Cycle 1; wherein the administration of the pharmaceutical composition alleviates or prevents a condition characterized by compromised white blood cell production (e.g., chemotherapy-induced neutropenia, or infection) in at least one of the 2 or more cycles (e.g., Cycle 1, or Cycles 1-2). In some embodiments, there is provided a method for treating cancer (e.g., breast cancer) in an individual (e.g., human), the method comprising: i) administering to the individual an effective amount of a chemotherapeutic agent for at least 4 cycles (e.g., 4 cycles) (e.g., the chemotherapeutic agent is administered on Day 1 of each cycle); and ii) administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent in Cycle 1; wherein the administration of the pharmaceutical composition alleviates or prevents a condition characterized by compromised white blood cell production (e.g., chemotherapy-induced neutropenia, or infection) in at least one of the 4 or more cycles (e.g., Cycle 1, or Cycles 1-4). In some embodiments, the G-CSF dimer pharmaceutical composition is administered at least 24 hours after (e.g., 24 hours after, or 48 hours after) administration of the chemotherapeutic agent in Cycle 1. In some embodiments, the method further comprises administering an effective amount of the G-CSF dimer pharmaceutical composition after 24 hours (e.g., at 24 hrs, or after any of 26, 28, 30, 32, 36, 40, 44, 48, 60 hrs, or more) of administration of the chemotherapeutic agent in each cycle starting Cycle 2, such as in each cycle of at least Cycles 2-4. In some embodiments, the G-CSF dimer pharmaceutical composition is administered after 48 hours (e.g., at 48 hrs, or after 48 hrs) of administration of the chemotherapeutic agent in each cycle starting Cycle 2, such as in each cycle of at least Cycles 2-4. In some embodiments, the chemotherapeutic agent is administered on Day 1 of each cycle (e.g., 2 or more days per cycle). In some embodiments, each cycle is about 21 days. In some embodiments, the administration time of the G-CSF dimer pharmaceutical composition is the same in each cycle starting Cycle 2, such as in each cycle of at least Cycles 2-4. In some embodiments, the administration time of the G-CSF dimer pharmaceutical composition is different in 2 or more cycles starting Cycle 2, such as different in 2 or more cycles of at least Cycles 2-4. In some embodiments, the condition characterized by compromised white blood cell production is chemotherapy-induced neutropenia. In some embodiments, the chemotherapeutic agent is a myelosuppressive chemotherapeutic agent. In some embodiments, the individual is administered two chemotherapeutic agents in each cycle, such as simultaneously (e.g., either in a same formulation, or in separate formulations). In some embodiments, the pharmaceutical composition comprises (or consists essentially of, or consists of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.2. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is administered at about 5 mg to about 25 mg (e.g., about 10 mg to about 25 mg, such as about 20 mg) of the G-CSF dimer for each administration. In some embodiments, the pharmaceutical composition is administered subcutaneously. In some embodiments, the pharmaceutical composition is contained (e.g., pre-filled) in a syringe (e.g., sterile and/or for single use). In some embodiments, the volume of the pharmaceutical composition within the syringe is about 1 mL. In some embodiments, the administration of the G-CSF dimer pharmaceutical composition achieves one or more of the following effects in one or more cycles (e.g., Cycle 1, Cycles 1-2, Cycles 2-4, or Cycles 1-4): i) reducing the duration of chemotherapy-induced neutropenia; ii) reducing or preventing the incidence of chemotherapy-induced neutropenia; iii) reducing or preventing the incidence of FN; iv) increasing ANC; v) activating neutrophil precursor cells; vi) activating myeloid stem cells; vii) activating mature neutrophils; viii) reducing the duration of an ANC of less than about 0.5×10⁹/L in the individual to less than about 36 hours; ix) preventing the ANC in the individual from reaching less than about 0.5×10⁹/L; x) increasing the ANC from the first occurrence of less than about 0.5×10⁹/L to greater than or equal to about 2.0×10⁹/L within about 10 days; and xi) increasing survival of the individual. In some embodiments, the chemotherapy-induced neutropenia is Grade 4 neutropenia.

In some embodiments, the individual is administered at least 2 (e.g., 2, 3, or 4) cycles of the chemotherapeutic agent, wherein the G-CSF dimer pharmaceutical composition is administered after (e.g., at least 24 hours after) administration of the chemotherapeutic agent in each cycle of the at least 2 cycles. In some embodiments, the individual is administered at least 4 cycles of the chemotherapeutic agent, wherein the G-CSF dimer pharmaceutical composition is administered after (e.g., at least 24 hours after) administration of the chemotherapeutic agent in each cycle of the at least 4 cycles. Hence in some embodiments, there is provided a method for treating or preventing a condition characterized by compromised white blood cell production (e.g., chemotherapy-induced neutropenia, or infection) in an individual (e.g., human, such as an individual having cancer) in need thereof, wherein the individual is administered at least 2 (e.g., 2, 3, or 4) cycles of a chemotherapeutic agent (e.g., the chemotherapeutic agent is administered on Day 1 of each cycle), and wherein the method comprises administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent in each cycle of the at least 2 cycles. In some embodiments, there is provided a method for treating or preventing a condition characterized by compromised white blood cell production (e.g., chemotherapy-induced neutropenia, or infection) in an individual (e.g., human, such as an individual having cancer) in need thereof, wherein the individual is administered at least 4 cycles (e.g., 4 cycles) of a chemotherapeutic agent (e.g., the chemotherapeutic agent is administered on Day 1 of each cycle), and wherein the method comprises administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent in each cycle of the at least 4 cycles. In some embodiments, the individual is administered the chemotherapeutic agent to treat cancer (e.g., breast cancer). In some embodiments, there is provided a method for treating cancer (e.g., breast cancer) in an individual (e.g., human, such as an individual having cancer), the method comprising: i) administering to the individual an effective amount of a chemotherapeutic agent for at least 2 (e.g., 2, 3, or 4) cycles (e.g., the chemotherapeutic agent is administered on Day 1 of each cycle); and ii) administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent in each cycle of the at least 2 cycles; wherein the administration of the pharmaceutical composition alleviates or prevents a condition characterized by compromised white blood cell production (e.g., chemotherapy-induced neutropenia, or infection) in at least one of the 2 or more cycles (e.g., Cycle 1, or Cycles 1-2). In some embodiments, there is provided a method for treating cancer (e.g., breast cancer) in an individual (e.g., human, such as an individual having cancer), the method comprising: i) administering to the individual an effective amount of a chemotherapeutic agent for at least 4 cycles (e.g., 4 cycles) (e.g., the chemotherapeutic agent is administered on Day 1 of each cycle); and ii) administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of the chemotherapeutic agent in each cycle of the at least 4 cycles; wherein the administration of the pharmaceutical composition alleviates or prevents a condition characterized by compromised white blood cell production (e.g., chemotherapy-induced neutropenia, or infection) in at least one of the 4 or more cycles (e.g., Cycle 1, or Cycles 1-4). In some embodiments, the chemotherapeutic agent is administered on Day 1 of each cycle (e.g., 2 or more days per cycle). In some embodiments, each cycle is about 21 days. In some embodiments, the administration time of the G-CSF dimer pharmaceutical composition is the same in each cycle of the at least 2 cycles. In some embodiments, the administration time of the G-CSF dimer pharmaceutical composition is different in each cycle of the at least 2 cycles. In some embodiments, the administration time of the G-CSF dimer pharmaceutical composition is the same in each cycle of the at least 4 cycles. In some embodiments, the administration time of the G-CSF dimer pharmaceutical composition is different in two or more cycles (e.g., 4) of the at least 4 cycles. In some embodiments, the G-CSF dimer pharmaceutical composition is administered at least 24 hours after (e.g., 24 hours after, or 48 hours after) administration of the chemotherapeutic agent in each cycle of the at least 2 cycles, such as in each cycle of the at least 4 cycles. In some embodiments, the condition characterized by compromised white blood cell production is chemotherapy-induced neutropenia. In some embodiments, the chemotherapeutic agent is a myelosuppressive chemotherapeutic agent. In some embodiments, the individual is administered two chemotherapeutic agents in each cycle, such as simultaneously (e.g., either in a same formulation, or in separate formulations). In some embodiments, the pharmaceutical composition comprises (or consists essentially of, or consists of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.2. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is administered at about 5 mg to about 25 mg (e.g., about 10 mg to about 25 mg, such as about 20 mg) of the G-CSF dimer for each administration. In some embodiments, the pharmaceutical composition is administered subcutaneously. In some embodiments, the pharmaceutical composition is contained (e.g., pre-filled) in a syringe (e.g., sterile and/or for single use). In some embodiments, the volume of the pharmaceutical composition within the syringe is about 1 mL. In some embodiments, the administration of the G-CSF dimer pharmaceutical composition achieves one or more of the following effects in one or more cycles (e.g., Cycle 1, Cycles 1-2, Cycles 2-4, or Cycles 1-4): i) reducing the duration of chemotherapy-induced neutropenia; ii) reducing or preventing the incidence of chemotherapy-induced neutropenia; iii) reducing or preventing the incidence of FN; iv) increasing ANC; v) activating neutrophil precursor cells; vi) activating myeloid stem cells; vii) activating mature neutrophils; viii) reducing the duration of an ANC of less than about 0.5×10⁹/L in the individual to less than about 36 hours; ix) preventing the ANC in the individual from reaching less than about 0.5×10⁹/L; x) increasing the ANC from the first occurrence of less than about 0.5×10⁹/L to greater than or equal to about 2.0×10⁹/L within about 10 days; and xi) increasing survival of the individual. In some embodiments, the chemotherapy-induced neutropenia is Grade 4 neutropenia.

In some embodiments, there is provided a method for treating or preventing a condition characterized by compromised white blood cell production (e.g., chemotherapy-induced neutropenia, or infection) in an individual (e.g., human) in need thereof, wherein the method comprises administering an effective amount of any of the pharmaceutical compositions described herein (e.g., G-CSF-Fc dimer pharmaceutical composition, such as efbemalenograstim alfa pharmaceutical composition) after administration of one or more chemotherapeutic agents in each cycle of at least 4 cycles, wherein the individual has breast cancer (e.g., metastatic or non-metastatic), and (i) wherein the individual is administered at least 4 cycles of docetaxel and cyclophosphamide, wherein docetaxel 75 mg/m² and cyclophosphamide 600 mg/m² is administered on Day 1 of each cycle, and wherein the pharmaceutical composition is administered on Day 2 of each cycle at least 24 hours after administration of docetaxel and cyclophosphamide; (ii) wherein the individual is administered at least 4 cycles of epirubicin and cyclophosphamide, wherein epirubicin 100 mg/m² and cyclophosphamide 600 mg/m² is administered on Day 1 of each cycle, and wherein the pharmaceutical composition is administered on Day 3 of each cycle at least 48 hours after administration of epirubicin and cyclophosphamide; or (iii) wherein the individual is administered at least 4 cycles of docetaxel and doxorubicin, wherein docetaxel 75 mg/m² and doxorubicin 60 mg/m² is administered on Day 1 of each cycle, and wherein the pharmaceutical composition is administered on Day 2 of each cycle at least 24 hours after administration of docetaxel and doxorubicin. In some embodiments, there is provided a method for treating breast cancer (e.g., metastatic or non-metastatic) in an individual (e.g., human) in need thereof, wherein the method comprises administering one or more chemotherapeutic agents in each cycle of at least 4 cycles, and further administering an effective amount of any of the pharmaceutical compositions described herein (e.g., G-CSF-Fc dimer pharmaceutical composition, such as efbemalenograstim alfa pharmaceutical composition) after administration of the one or more chemotherapeutic agents in each cycle of the at least 4 cycles, wherein the administration of the pharmaceutical composition alleviates or prevents a condition characterized by compromised white blood cell production (e.g., chemotherapy-induced neutropenia, or infection); and (i) wherein the individual is administered at least 4 cycles of docetaxel and cyclophosphamide, wherein docetaxel 75 mg/m² and cyclophosphamide 600 mg/m² is administered on Day 1 of each cycle, and wherein the pharmaceutical composition is administered on Day 2 of each cycle at least 24 hours after administration of docetaxel and cyclophosphamide; (ii) wherein the individual is administered at least 4 cycles of epirubicin and cyclophosphamide, wherein epirubicin 100 mg/m² and cyclophosphamide 600 mg/m² is administered on Day 1 of each cycle, and wherein the pharmaceutical composition is administered on Day 3 of each cycle at least 48 hours after administration of epirubicin and cyclophosphamide; or (iii) wherein the individual is administered at least 4 cycles of docetaxel and doxorubicin, wherein docetaxel 75 mg/m² and doxorubicin 60 mg/m² is administered on Day 1 of each cycle, and wherein the pharmaceutical composition is administered on Day 2 of each cycle at least 24 hours after administration of docetaxel and doxorubicin. In some embodiments, each cycle is about 21 days. In some embodiments, the pharmaceutical composition comprises (or consists essentially of, or consists of): (a) about 20 mg/mL of a G-CSF dimer (e.g., G-CSF-Fc dimer, such as efbemalenograstim alfa); (b) about 10 mM sodium acetate; (c) about 1 mM EDTA; (d) about 5% (w/v) sorbitol; and (e) about 0.01% (w/v) polysorbate 20; wherein the pH of the pharmaceutical composition is about 5.2. In some embodiments, the G-CSF dimer comprises two monomeric subunits each comprising (or consisting essentially of, or consisting of) an amino acid sequence of any of SEQ ID NOs: 6-10 (e.g., SEQ ID NO: 6). In some embodiments, the G-CSF dimer is efbemalenograstim alfa. In some embodiments, the pharmaceutical composition is administered at about 5 mg to about 25 mg (e.g., about 10 mg to about 25 mg, such as about 20 mg) of the G-CSF dimer for each administration. In some embodiments, the pharmaceutical composition is administered subcutaneously. In some embodiments, the pharmaceutical composition is contained (e.g., pre-filled) in a syringe (e.g., sterile and/or for single use). In some embodiments, the volume of the pharmaceutical composition within the syringe is about 1 mL.

In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, induce a rise (e.g., increasing at least about any of 5%, 10%, 20%, 30%, 40%, 50%, 60%, 70%, 80%, 90%, 95%, 1-fold, 2-fold, 5-fold, 10-fold, 50-fold, or more) in white blood cells (WBC) or reduce (e.g., reducing at least about any of 5%, 10%, 20%, 30%, 40%, 50%, 60%, 70%, 80%, 90%, 95%, or more) a loss of WBC in the individual. For example, in some embodiments, the number of neutrophils (e.g., ANC) is increased (e.g., increasing at least about any of 5%, 10%, 20%, 30%, 40%, 50%, 60%, 70%, 80%, 90%, 95%, 1-fold, 2-fold, 5-fold, 10-fold, 20-fold, or more) in the individual. In some embodiments, the decrease in the number of neutrophils is inhibited (e.g., inhibiting at least about any of 5%, 10%, 20%, 30%, 40%, 50%, 60%, 70%, 80%, 90%, 95%, or more) in the individual. In some embodiments, the nadir ANC is increased (e.g., increasing at least about any of 5%, 10%, 20%, 30%, 40%, 50%, 60%, 70%, 80%, 90%, 95%, 1-fold, 2-fold, 5-fold, 10-fold, or more) in the individual. In some embodiments, the recovery ANC is increased (e.g., increasing at least about any of 5%, 10%, 20%, 30%, 40%, 50%, 60%, 70%, 80%, 90%, 95%, 1-fold, 2-fold, 5-fold, 10-fold, 20-fold, or more) in the individual. In some embodiments, the time to ANC recovery is reduced (e.g., reducing at least about any of 5%, 10%, 20%, 30%, 40%, 50%, 60%, 70%, 80%, 90%, 95%, or more) in the individual. In some embodiments, the one or more effects described herein is achieved in Cycle 1 of the individual's chemotherapy. In some embodiments, the one or more effects described herein is achieved in one or more cycles of the individual's chemotherapy, e.g., Cycles 1-2 of 2 or more cycles, or Cycles 1-3 of 4 or more cycles. In some embodiments, the one or more effects described herein is achieved in each cycle (e.g., all 2 cycles, or all 4 cycles) of the individual's chemotherapy.

In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, reduce (e.g., reducing at least about any of 5%, 10%, 20%, 30%, 40%, 50%, 60%, 70%, 80%, 90%, 95%, or more) the duration of chemotherapy-induced neutropenia (e.g., Grade 4) in the individual. In some embodiments, the duration of chemotherapy-induced neutropenia is reduced for one or more grades of neutropenia, such as Grade I, Grade II, Grade III, or Grade IV. In some embodiments, the methods described herein reduce the duration of Grade 4 (severe) neutropenia, or reduce the duration of an ANC of less than about 0.5×10⁹/L, such as reducing to less than about 36 hours, e.g., reducing to less than about any of 34, 32, 30, 28, 26, 24, 23, 22, 21, 20, 19, 18, 17, 16, 15, 14, 13, 12, 11, 10, 9, 8, 7, 6, 5, 4, 3, 2, 1, 0.5, 0.1 hours, or less. In some embodiments, the methods described herein reduce the duration of Grade 4 (severe) neutropenia, or reduce the duration of an ANC of less than about 0.5×10⁹/L, to less than about 36 hours. In some embodiments, the duration of Grade 4 (severe) neutropenia (ANC <0.5×10⁹/L) is the number of days in which an individual has had an ANC <0.5×10⁹/L in Cycle 1 of the individual's chemotherapy. In some embodiments, the methods described herein reduce the duration of Grade 4 neutropenia, or reduce the duration of an ANC of less than about 0.5×10⁹/L, in Cycle 1 of the individual's chemotherapy. In some embodiments, the methods described herein reduce the duration of Grade 4 neutropenia, or reduce the duration of an ANC of less than about 0.5×10⁹/L, in one or more cycles of the individual's chemotherapy, e.g., Cycles 1-2 of 2 or more cycles, or Cycles 1-3 of 4 or more cycles. In some embodiments, the methods described herein reduce the duration of Grade 4 neutropenia, or reduce the duration of an ANC of less than about 0.5×10⁹/L, in each cycle (e.g., all 2 cycles, or all 4 cycles) of the individual's chemotherapy. In some embodiments, the methods described herein reduce the duration of Grade 3 neutropenia, or reduce the duration of an ANC of less than about 1.0×10⁹/L, such as reducing to less than about 36 hours, e.g., reducing to less than about any of 34, 32, 30, 28, 26, 24, 23, 22, 21, 20, 19, 18, 17, 16, 15, 14, 13, 12, 11, 10, 9, 8, 7, 6, 5, 4, 3, 2, 1, 0.5, 0.1 hours, or less. In some embodiments, the duration of Grade 3 neutropenia (ANC <1.0×10⁹/L) is the number of days in which an individual has had an ANC <1.0×10⁹/L in Cycle 1 of the individual's chemotherapy. In some embodiments, the methods described herein reduce the duration of Grade 3 neutropenia, or reduce the duration of an ANC of less than about 1.0×10⁹/L, in Cycle 1 of the individual's chemotherapy. In some embodiments, the methods described herein reduce the duration of Grade 3 neutropenia, or reduce the duration of an ANC of less than about 1.0×10⁹/L, in one or more cycles of the individual's chemotherapy, e.g., Cycles 1-2 of 2 or more cycles, or Cycles 1-3 of 4 or more cycles. In some embodiments, the methods described herein reduce the duration of Grade 3 neutropenia, or reduce the duration of an ANC of less than about 1.0×10⁹/L, in each cycle (e.g., all 2 cycles, or all 4 cycles) of the individual's chemotherapy.

In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, reduce (e.g., reducing at least about any of 5%, 10%, 20%, 30%, 40%, 50%, 60%, 70%, 80%, 90%, 95%, or more) or prevent the incidence of chemotherapy-induced neutropenia (e.g., Grade 4) in the individual. In some embodiments, the incidence of chemotherapy-induced neutropenia is reduced for one or more grades of neutropenia, such as Grade I, Grade II, Grade III, or Grade IV. In some embodiments, the incidence of chemotherapy-induced neutropenia is reduced or prevented for any grade of neutropenia. In some embodiments, the methods described herein reduce or prevent the incidence of Grade 4 (severe) neutropenia. In some embodiments, the methods described herein prevent the ANC in the individual from reaching less than about 0.5×10⁹/L. In some embodiments, the methods described herein reduce the incidence of Grade 4 neutropenia, or reduce the incidence of an ANC reaching less than about 0.5×10⁹/L, at least once, such as at least 2, 3, 4, or more times. In some embodiments, the methods described herein reduce or prevent the incidence of Grade 4 neutropenia, or reduce or prevent the incidence of an ANC from reaching less than about 0.5×10⁹/L, in Cycle 1 of the individual's chemotherapy. In some embodiments, the methods described herein reduce or prevent the incidence of Grade 4 neutropenia, or reduce or prevent the incidence of an ANC from reaching less than about 0.5×10⁹/L, in one or more cycles of the individual's chemotherapy, e.g., Cycles 1-2 of 2 or more cycles, or Cycles 1-3 of 4 or more cycles. In some embodiments, the methods described herein reduce or prevent the incidence of Grade 4 neutropenia, or reduce or prevent the incidence of an ANC from reaching less than about 0.5×10⁹/L, in each cycle (e.g., all 2 cycles, or all 4 cycles) of the individual's chemotherapy. In some embodiments, the methods described herein reduce or prevent the incidence of Grade 3 neutropenia. In some embodiments, the methods described herein prevent the ANC in the individual from reaching less than about 1.0×10⁹/L. In some embodiments, the methods described herein reduce the incidence of Grade 3 neutropenia, or reduce the incidence of an ANC reaching less than about 1.0×10⁹/L, at least once, such as at least 2, 3, 4, or more times. In some embodiments, the methods described herein reduce or prevent the incidence of Grade 3 neutropenia, or reduce or prevent the incidence of an ANC from reaching less than about 1.0×10⁹/L, in Cycle 1 of the individual's chemotherapy. In some embodiments, the methods described herein reduce or prevent the incidence of Grade 3 neutropenia, or reduce or prevent the incidence of an ANC from reaching less than about 1.0×10⁹/L, in one or more cycles of the individual's chemotherapy, e.g., Cycles 1-2 of 2 or more cycles, or Cycles 1-3 of 4 or more cycles. In some embodiments, the methods described herein reduce or prevent the incidence of Grade 3 neutropenia, or reduce or prevent the incidence of an ANC from reaching less than about 1.0×10⁹/L, in each cycle (e.g., all 2 cycles, or all 4 cycles) of the individual's chemotherapy.

In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, reduce (e.g., reducing at least about any of 5%, 10%, 20%, 30%, 40%, 50%, 60%, 70%, 80%, 90%, 95%, or more) or prevent the incidence of febrile neutropenia (FN) in the individual. FN is defined as a single oral temperature of ≥38.3° C. (101° F.) or a temperature of ≥38.0° C. (100.4° F.) sustained for >1 hour and ANC <0.5×10⁹/L on the same day. Hence in some embodiments, the methods described herein reduce or prevent the incidence of a single oral temperature of ≥38.3° C. (101° F.) or a temperature of >38.0° C. (100.4° F.) sustained for more than 1 hour and an ANC <0.5×10⁹/L on the same day (e.g., within less than about any of 24, 20, 18, 16, 14, 12, 10, 8, 6, 4, 2, 1, 0.5 hr, or less) in an individual. In some embodiments, the methods described herein reduce the incidence of FN at least once, such as at least 2, 3, 4, or more times. In some embodiments, the methods described herein reduce or prevent the incidence of FN in Cycle 1 of the individual's chemotherapy. In some embodiments, the methods described herein reduce or prevent the incidence of FN in one or more cycles of the individual's chemotherapy, e.g., Cycles 1-2 of 2 or more cycles, or Cycles 1-3 of 4 or more cycles. In some embodiments, the methods described herein reduce or prevent the incidence of FN in each cycle (e.g., all 2 cycles, or all 4 cycles) of the individual's chemotherapy.

In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, reduce (e.g., reducing at least about any of 5%, 10%, 20%, 30%, 40%, 50%, 60%, 70%, 80%, 90%, 95%, or more) the duration and/or incidence, or prevent the incidence, of an infection (e.g., infection manifested by neutropenia, such as FN) in the individual. In some embodiments, the methods described herein reduce (e.g., reducing at least about any of 5%, 10%, 20%, 30%, 40%, 50%, 60%, 70%, 80%, 90%, 95%, or more) the duration and/or incidence of using intravenous antibiotics. In some embodiments, the methods described herein reduce the incidence of an infection at least once, such as at least 2, 3, 4, or more times. In some embodiments, the methods described herein reduce the duration and/or incidence, or prevent the incidence, of an infection in Cycle 1 of the individual's chemotherapy. In some embodiments, the methods described herein reduce the duration and/or incidence, or prevent the incidence, of an infection in one or more cycles of the individual's chemotherapy, e.g., Cycles 1-2 of 2 or more cycles, or Cycles 1-3 of 4 or more cycles. In some embodiments, the methods described herein reduce the duration and/or incidence, or prevent the incidence, of an infection in each cycle (e.g., all 2 cycles, or all 4 cycles) of the individual's chemotherapy.

In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, reduce (e.g., reducing at least about any of 5%, 10%, 20%, 30%, 40%, 50%, 60%, 70%, 80%, 90%, 95%, or more) the duration and/or incidence, or prevent the incidence, of hospitalization for febrile neutropenia or an infection in the individual. In some embodiments, the methods described herein reduce (e.g., reducing at least about any of 5%, 10%, 20%, 30%, 40%, 50%, 60%, 70%, 80%, 90%, 95%, or more) the duration and/or incidence of using intravenous antibiotics. In some embodiments, the methods described herein reduce the incidence of hospitalization for FN or an infection at least once, such as at least 2, 3, 4, or more times. In some embodiments, the methods described herein reduce the duration and/or incidence, or prevent the incidence, of hospitalization for FN or an infection in Cycle 1 of the individual's chemotherapy. In some embodiments, the methods described herein reduce the duration and/or incidence, or prevent the incidence, of hospitalization for FN or an infection in one or more cycles of the individual's chemotherapy, e.g., Cycles 1-2 of 2 or more cycles, or Cycles 1-3 of 4 or more cycles. In some embodiments, the methods described herein reduce the duration and/or incidence, or prevent the incidence, of hospitalization for FN or an infection in each cycle (e.g., all 2 cycles, or all 4 cycles) of the individual's chemotherapy.

In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, reduce (e.g., reducing at least about any of 5%, 10%, 20%, 30%, 40%, 50%, 60%, 70%, 80%, 90%, 95%, or more) the depth of the ANC nadir, or increase (e.g., increasing at least about any of 5%, 10%, 20%, 30%, 40%, 50%, 60%, 70%, 80%, 90%, 95%, 1-fold, 2-fold, 5-fold, 10-fold, or more) the ANC nadir value, in the individual. In some embodiments, the methods described herein reduce the depth of the ANC nadir, or increase the ANC nadir value, in Cycle 1 of the individual's chemotherapy. In some embodiments, the methods described herein reduce the depth of the ANC nadir, or increase the ANC nadir value, in one or more cycles of the individual's chemotherapy, e.g., Cycles 1-2 of 2 or more cycles, or Cycles 1-3 of 4 or more cycles. In some embodiments, the methods described herein reduce the depth of the ANC nadir, or increase the ANC nadir value, in each cycle (e.g., all 2 cycles, or all 4 cycles) of the individual's chemotherapy.

In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, reduce (e.g., reducing at least about any of 5%, 10%, 20%, 30%, 40%, 50%, 60%, 70%, 80%, 90%, 95%, or more) the time to ANC recovery in the individual. In some embodiments, the time to ANC recovery is reduced at least about any of 5 minutes, 30 minutes, 1 hour, 2 hours, 0.1, 0.2, 0.3, 0.4, 0.5, 0.6, 0.7, 0.8, 0.9, 1, 2, 3, 4, 5, 6, 7, 8, 9, 10 days, or more. In some embodiments, ANC recovery is reaching an ANC ≥2.0×10⁹/L after an ANC nadir (e.g., expected ANC nadir). Hence in some embodiments, the methods described herein reduce the time to reach an ANC ≥2.0×10⁹/L after an ANC nadir, such as the first occurrence of an ANC of less than about 0.5×10⁹/L. In some embodiments, the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, increases the ANC from the first occurrence of less than about 0.5×10⁹/L to greater than or equal to about 2.0×10⁹/L within about 10 days, such as within about any of 9, 8, 7, 6, 5, 4, 3, 2, 1, 0.5, 0.2, 0.1 day, or less. In some embodiments, the methods described herein increase the ANC from the first occurrence of less than about 0.5×10⁹/L to greater than or equal to about 1.5×10⁹/L within about 10 days, such as within about any of 9, 8, 7, 6, 5, 4, 3, 2, 1, 0.5, 0.2, 0.1 day, or less. In some embodiments, the methods described herein reduce the time to ANC recovery in Cycle 1 of the individual's chemotherapy. In some embodiments, the methods described herein reduce the time to ANC recovery in one or more cycles of the individual's chemotherapy, e.g., Cycles 1-2 of 2 or more cycles, or Cycles 1-3 of 4 or more cycles. In some embodiments, the methods described herein reduce the time to ANC recovery in each cycle (e.g., all 2 cycles, or all 4 cycles) of the individual's chemotherapy.

In some embodiments, the one or more effects (e.g., therapeutic effects) achieved by the methods described herein, or the administration of the G-CSF dimer pharmaceutical composition (e.g., G-CSF-Fc dimer pharmaceutical composition, such as efbemalenograstim alfa pharmaceutical composition) after (e.g., at least 24 hours after) administration of a chemotherapeutic agent to an individual, is compared to that of an individual not administered with a G-CSF dimer pharmaceutical composition (such as any of the G-CSF dimer pharmaceutical compositions described herein). In some embodiments, the one or more effects achieved by the methods described herein is compared to that of an individual administered with pegfilgrastim (e.g., Neulasta®) or biosimilars, eflapegrastim (e.g., Rolontis®, HM10460A; or ROLVEDON™) or biosimilars, or filgrastim (GRAN®) or biosimilars, with the same treatment schedule as the G-CSF dimer pharmaceutical composition, i.e., after (e.g., 24 hours after, or 48 hours after) administration of a same chemotherapeutic agent. In some embodiments, the one or more effects achieved by the methods described herein is compared to that of an individual administered with pegfilgrastim (e.g., Neulasta®) or biosimilars, eflapegrastim (e.g., Rolontis®, HM10460A; or ROLVEDON™) or biosimilars, or filgrastim (GRAN®) or biosimilars, with equivalent G-CSF doses as the G-CSF dimer pharmaceutical composition (e.g., dosing schedule can be different). In some embodiments, the methods using the G-CSF dimer pharmaceutical compositions described herein: i) achieve comparable (e.g., within about 5% difference) or better therapeutic effects, and/or ii) have comparable (e.g., within about 5% difference) or less adverse events, compared to that of an individual administered with pegfilgrastim (e.g., Neulasta®) or biosimilars, eflapegrastim (e.g., Rolontis®, HM10460A; or ROLVEDON™) or biosimilars, or filgrastim (GRAN®) or biosimilars with the same treatment schedule and/or equivalent G-CSF doses as the G-CSF dimer pharmaceutical composition.

In some embodiments, the individual is a human. In some embodiments, the G-CSF dimer pharmaceutical composition (e.g., G-CSF-Fc dimer pharmaceutical composition, such as efbemalenograstim alfa pharmaceutical composition) is administered at about 5 mg to about 25 mg for each administration, such as any of about 10 mg to about 25 mg, about 5 mg to about 15 mg, about 5 mg to about 20 mg, about 10 mg to about 15 mg, about 10 mg to about 20 mg, about 15 mg to about 25 mg, about 15 mg to about 20 mg, or about 18 mg to about 22 mg, for each administration. In some embodiments, the G-CSF dimer pharmaceutical composition is administered at about any of 5, 6, 7, 8, 9, 10, 11, 12, 13, 14, 15, 16, 17, 18, 19, 20, 21, 22, 23, 24, or 25 mg, or any values in between, for each administration. In some embodiments, the G-CSF dimer pharmaceutical composition is administered at about 20 mg for each administration. In some embodiments, the individual is a non-human animal. In some embodiments, the G-CSF dimer pharmaceutical composition is administered to the non-human animal at an equivalent human dose. In some embodiments, when the individual is administered with 2 or more cycles of chemotherapeutic agent, the G-CSF dimer pharmaceutical composition is administered at the same amount (e.g., 20 mg) for each cycle. In some embodiments, when the individual is administered with 2 or more cycles of chemotherapeutic agent, the G-CSF dimer pharmaceutical composition is administered at different amount for at least 2 of the cycles.

In some embodiments, the individual to be treated, or the individual administered with the G-CSF dimer pharmaceutical composition (e.g., G-CSF-Fc dimer pharmaceutical composition, such as efbemalenograstim alfa pharmaceutical composition), meets one or more of the following criteria: i) is at least 18 years old for a human; ii) is a female for an individual having breast cancer (e.g., Stage I-III breast cancer); iii) is scheduled to undergo 2 or more (e.g., 2, 3, 4, or more) cycles of chemotherapy (e.g., neoadjuvant or adjuvant); iv) has Eastern Cooperative Oncology Group (ECOG) Performance status of ≤2; v) has ANC ≥2.0×10⁹/L, hemoglobin ≥11.0 g/dL, and a platelet count ≥100×10⁹/L before the administration of the chemotherapeutic agent; vi) has adequate renal, hepatic, and cardiac function (e.g., alanine aminotransferase (ALT), aspartate aminotransferase (AST), and/or alkaline phosphatase are less than 2.5× the upper limits of normal (ULN); total bilirubin and/or serum creatinine is less than 1.5×ULN); vii) under contraception at least one month prior to and during the administration of the chemotherapeutic agent. In some embodiments, the individual does not have latex allergy. In some embodiments, the individual does not have serious allergic reactions to efbemalenograstim alfa. In some embodiments, the methods described herein further comprise selecting an individual meeting the one or more of the above criteria.

In some embodiments, the pharmaceutical composition is administered at a dosage of at least about 0.01 mg/kg, such as at least about any of 0.02 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, or 1 mg/kg. In some embodiments, the pharmaceutical composition is administered at a dosage of from about 0.01 mg/kg to about 1 mg/kg, such as any of from about 0.02 mg/kg to about 0.9 mg/kg, from about 0.05 mg/kg to about 0.8 mg/kg, or from about 0.1 mg/kg to about 0.7 mg/kg. In some embodiments, the pharmaceutical composition is administered at a dosage of about 0.01 mg/kg, about 0.02 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, or about 1 mg/kg.

The pharmaceutical composition may be administered at any desired regimen and frequency. In some embodiments according to any of the methods described herein, the pharmaceutical composition is administered once every day, once every 2 days, once every 3 days, once every 4 days, once every 5 days, once every 6 days, once every week, once every two weeks, once every three weeks, once every four weeks, once every 6 weeks, or once every 8 weeks. In some embodiments, the pharmaceutical composition is administered once every four weeks. In some embodiments, the pharmaceutical composition is administered once every 3 weeks. In some embodiments, the pharmaceutical composition is administered at about 20 mg per administration, such as about 20 mg once per chemotherapy cycle. In some embodiments, the method comprises administering the pharmaceutical composition as a single dose.

In some embodiments, the pharmaceutical composition is administered using a syringe (e.g., a pre-filled syringe), such as any of the syringes described herein. In some embodiments, the pharmaceutical composition is administered subcutaneously. In some embodiments, the pharmaceutical composition is contained (e.g., pre-filled) in a syringe. In some embodiments, the volume of the pharmaceutical composition within the syringe is about 1 mL. In some embodiments, the syringe contains about 20 mg of a G-CSF dimer, such as any of the G-CSF dimers described herein (e.g., G-CSF-Fc dimer, such as efbemalenograstim alfa). In some embodiments, the syringe is sterile. In some embodiments, the syringe is for single use.

The pharmaceutical composition can be administered to an individual (e.g., human, such as an individual having cancer) via various routes, including, for example, intravenous, intra-arterial, intraperitoneal, intravascular, intramuscular, subcutaneous, transmucosal, and transdermal. In some embodiments, sustained continuous release formulation of the pharmaceutical composition may be used. In some embodiments, the implantation of a slow-release device, e.g., a mini-osmotic pump, can be used. Other modes of delivery include, but are not limited to, the use of liposomal formulations, or intravenous infusion. In some embodiments, the pharmaceutical composition is administered intravenously. In some embodiments, the pharmaceutical composition is administered intraperitoneally. In some embodiments, the pharmaceutical composition is administered intramuscularly. In some embodiments, the pharmaceutical composition is administered subcutaneously. When administered subcutaneously, such pharmaceutical compositions are typically administered in a volume of less than about 2.0 mL, such as about 1.5 mL, about 1 mL, or about 0.5 mL per injection site. In some embodiments, the injection volume is 1 mL.

### Neutropenia

Neutropenia is characterized by a neutrophil count in the peripheral blood of lower than 1.8×10⁹/L for an adult and 1.5×10⁹/L for a child. Neutropenia is often a precursor of infection: the lower the neutrophil count is, the higher the risk of infection is.

The guideline used to classify neutropenia is shown in Table A. The frequency and severity of infection caused by neutropenia are also influenced by other factors, such as: the integrity of the mucosa and skin, immunoglobulin, lymphocytes, monocytes, the function and level of the complement system, etc.

TABLE A
Neutropenia classification

| | | Risk of |
|---|---|---|
| Neutropenia | Neutrophil Count | Infection |
| Mild | 1.0 × 10⁹/L to 1.8 × 10⁹/L | Minimal |
| Moderate | 0.5 × 10⁹/L to 1.0 × 10⁹/L | Increasing |
| Severe | <0.5 × 10⁹/L | Severe |

According to the cause of neutropenia, the common clinical neutropenia can be divided into the following categories: disorder of hematopoietic system generation that are caused by secondary factors such as drugs, radiation, chemical reagents and infection; changes of in vivo distribution and circulation, increased utilization and turnover. The severity of chemotherapy-induced neutropenia in tumor patients generally depends on the dosage of chemotherapy, and the repeated use of chemotherapy may have a cumulative effect on neutropenia. A main clinical consequence of neutropenia is infected complication. Most of the infections in those patients are mainly caused by aerobic bacteria, including Gram-negative bacteria (Escherichia coli, Klebsiella pheumoniae and Pseudomonas aeruginosa), Gram-positive bacteria (Staphylococci, α-hemolytic Streptococci, and Straphylococcus aureus) and fungi.

Cytotoxic chemotherapy is still one of the major treatments of cancer. The biggest disadvantage of chemotherapy treatment is that this treatment would indiscriminately kill healthy cells with rapid proliferation and differentiation together with tumor cells. The toxicity caused by chemotherapy is mainly reflected in the hematopoietic system, which is clinically known as chemotherapy-induced neutropenia.

Neutropenia may delay the next treatment cycle, which directly impacts on the therapeutic effects of chemotherapy. A severe neutropenia, i.e., the ANC is lower than 0.5×10⁹/L, can cause infection in patient, organ failure and even threaten the life of the patient.

In some embodiments, the neutropenia is a Grade 1 neutropenia. In some embodiments, the neutropenia is a Grade 2 neutropenia. In some embodiments, the neutropenia is a Grade 3 neutropenia. In some embodiments, the neutropenia is a Grade 4 neutropenia or severe neutropenia. In some embodiments, the neutropenia is a febrile neutropenia (FN).

Hence in some embodiments, there is provided a method for treating or preventing neutropenia in an individual (e.g., human, such as a human having cancer) in need thereof, the method comprising administering to the individual an effective amount of any of the pharmaceutical compositions described herein (e.g., G-CSF-Fc dimer pharmaceutical composition, such as efbemalenograstim alfa pharmaceutical composition) after (e.g., at least 24 hours after) administration of an agent that induces neutropenia, such as a chemotherapeutic agent that can induce neutropenia.

### Diseases to be Treated by Chemotherapeutic Agents

In some embodiments, the individual is administered the chemotherapeutic agent to treat cancer.

The cancer that can be treated include but is not limited to: colorectal cancer, breast cancer, gastric cancer, prostate cancer, ovarian cancer, cervical cancer, melanoma, liver cancer, head and neck cancer, glioma, gallbladder cancer, pancreatic cancer, prostate adenocarcinoma, ampullary cancer, esophageal cancer, renal cancer, thyroid cancer, squamous cell carcinoma, lung cancer, B cell lymphomas, acute lymphoblastic leukemia (ALL), chronic lymphocytic leukemia (CLL), Non-Hodgkin's Lymphoma (NHL), Burkitt lymphoma, Wilms' tumor, etc. In some embodiments, the cancer is a solid cancer. In some embodiments, the cancer is a liquid cancer.

In some embodiments, the cancer is a non-myeloid cancer, e.g., non-myeloid malignant tumor. In some embodiments, the cancer is selected from the group consisting of breast cancer, non-small cell lung cancer, small cell lung cancer, ovarian cancer, sarcoma, urothelial cancer, germ cell tumor, and non-Hodgkin's lymphoma. In some embodiments, the cancer is breast cancer. In some embodiments, the cancer is Stage I-III, or Stage II-IV breast cancer. In some embodiments, the breast cancer is metastatic breast cancer. In some embodiments, the breast cancer is non-metastatic breast cancer.

In some embodiments, the methods of administering G-CSF dimer pharmaceutical compositions described herein ensure that the individual does not delay the treatment schedule of the chemotherapeutic agent, or ensure that the delay in the treatment schedule of the chemotherapeutic agent is no more than about 5 days, such as no more than about any of 4.5, 4, 3.5, 3, 2.5, 2, 1.8, 1.6, 1.4, 1.2, 1, 0.8, 0.6, 0.4, 0.2, 0.1 days, or less. In some embodiments, when the chemotherapeutic agent is to be administered for 2 or more cycles, the methods of administering G-CSF dimer pharmaceutical compositions described herein ensure that the individual does not delay the treatment schedule of the chemotherapeutic agent within each cycle, or the delay is only in Cycle 2 (e.g., no more than about 5 days), or the delay is not in all cycles (e.g., only Cycles 2 and 3 of 4 cycles).

### Chemotherapeutic Agents

In some embodiments, the chemotherapeutic agent is a myelosuppressive chemotherapeutic agent. In some embodiments, the myelosuppressive chemotherapeutic agent is selected from the group consisting of epirubicin, docetaxel, cyclophosphamide, doxorubicin, etoposide, cisplatin, paclitaxel, topotecan, vincristine, methylprednisolone, cytarabine, and a combination thereof.

In some embodiments, the cancer to be treated by the chemotherapeutic agent is breast cancer. In some embodiments, the individual is administered two or more chemotherapeutic agents comprising: i) epirubicin and cyclophosphamide (e.g., epirubicin 100 mg/m² and cyclophosphamide 600 mg/m²); ii) docetaxel and cyclophosphamide (e.g., docetaxel 75 mg/m² and cyclophosphamide 600 mg/m²); iii) doxorubicin and cyclophosphamide; iv) docetaxel and doxorubicin (e.g., 75 mg/m² docetaxel and 60 mg/m² doxorubicin); or v) docetaxel, doxorubicin, and cyclophosphamide. In some embodiments, the two or more chemotherapeutic agents are administered simultaneously, either in a same formulation, or in separate formulations. In some embodiments, the two or more chemotherapeutic agents are administered sequentially.

In some embodiments, the chemotherapeutic agent induces neutropenia (e.g., any grade of neutropenia), such as Grade 4 neutropenia. In some embodiments, the chemotherapeutic agent induces FN. In some embodiments, the chemotherapeutic agent reduces ANC to less than about 0.5×10⁹/L. In some embodiments, the chemotherapeutic agent reduces ANC, e.g., reducing at least about any of 5%, 10%, 20%, 30%, 40%, 50%, 60%, 70%, 80%, 90%, 95%, or more ANC. In some embodiments, the chemotherapeutic agent induces a duration of neutropenia (DN) of at least about 2 days, such as at least about any of 3, 4, 5, 6, 7, 8, 9, 10 days, or more.

### VII. Systems, Kits, and Methods of Preparation

Also provided are systems, kits, and methods of preparation of the pharmaceutical compositions described herein (e.g., efbemalenograstim alfa pharmaceutical composition).

In some embodiments, there is provided a lyophilized pharmaceutical composition. Lyophilized pharmaceutical compositions adapted for subcutaneous administration are described in WO97/04801. Such lyophilized pharmaceutical compositions may be reconstituted with a suitable diluent to a high protein concentration and the reconstituted formulation may be administered subcutaneously to the individual to be imaged, diagnosed, or treated herein.

The pharmaceutical compositions to be used for in vivo administration must be sterile. This is readily accomplished by, e.g., filtration through sterile filtration membranes. In some embodiments, there is provided a syringe containing any one of the pharmaceutical compositions described herein (e.g., efbemalenograstim alfa pharmaceutical composition). In some embodiments, the syringe is sterile. In some embodiments, the syringe is for single use. In some embodiments, the syringe is pre-filled with about 1 mL of the pharmaceutical composition described herein.

Also provided are kits comprising any one of the pharmaceutical compositions described herein (e.g., efbemalenograstim alfa pharmaceutical composition). The kits may be useful for any of the methods of treatment or prevention of a disease or condition described herein.

In some embodiments, there is provided a kit comprising a G-CSF dimer (e.g., G-CSF-Fc dimer, such as efbemalenograstim alfa). In some embodiments, the kit further comprises other components necessary for making a G-CSF dimer pharmaceutical composition, such as any of the pharmaceutical compositions described herein.

In some embodiments, the kit further comprises a device capable of delivering the pharmaceutical composition into an individual. One type of device, for applications such as parenteral delivery, is a syringe that is used to inject the pharmaceutical composition into the body of a subject.

In some embodiments, the kit further comprises one or more therapeutic agents for treating a disease or condition, e.g., cancer. In some embodiments, the therapeutic agent is a chemotherapeutic agent.

The kits of the present application can be in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle). The label or package insert indicates that the composition is used for imaging, diagnosing, or treating a particular condition in an individual. The label or package insert will further comprise instructions for administering the composition to the individual and/or for imaging the individual. The label may indicate directions for reconstitution and/or use. The container holding the composition may be a multi-use vial, which allows for repeat administrations (e.g., from 2-6 administrations) of the reconstituted formulation. Package insert refers to instructions customarily included in commercial packages of diagnostic or therapeutic products that contain information about the indications, usage, dosage, administration, contraindications and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the compositions and instructions for use, packaged in quantities sufficient for storage and use in pharmacies, for example, hospital pharmacies and compounding pharmacies.

In some embodiments, there is provided a method of manufacturing any of the pharmaceutical compositions described herein, comprising one or more (e.g., all) of the following steps: (i) culturing a host cell under a condition suitable for expressing a G-CSF dimer (e.g., any of the G-CSF dimers described herein, such as efbemalenograstim alfa); (ii) isolating the expressed G-CSF dimer from the cell culture; (iii) purifying the expressed G-CSF dimer; and (iv) formulating the purified G-CSF dimer with the buffering agent, the stabilizing agent, the tonicity agent, and the surfactant. In some embodiments, the purification step comprises one or more of: affinity chromatography, viral inactivation, ion exchange chromatography, mixed-mode chromatography, and filtration. In some embodiments, there is provided a method of manufacturing any of the pharmaceutical compositions described herein, comprising formulating a purified G-CSF dimer with a buffering agent, a stabilizing agent, a tonicity agent, and a surfactant, such as those described herein. In some embodiments, the method further comprises adjusting the pH of the pharmaceutical composition. In some embodiments, the method further comprises filling the pharmaceutical composition into a syringe. Also see Example 7 and Table 13 for exemplary methods.

The present application also provides isolated nucleic acids encoding any of the G-CSF dimers described herein, vectors and host cells comprising such isolated nucleic acids, and recombinant methods for the production of the G-CSF dimer. In some embodiments, the nucleic acid encoding the G-CSF dimer comprises the sequence of SEQ ID NO: 11.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### VIII. Exemplary Embodiments

In some embodiments, there is provided a pharmaceutical composition comprising (or consisting essentially of, or consisting of): a) about 1 mg/mL to about 100 mg/mL of a G-CSF dimer, wherein the G-CSF dimer comprises two monomeric subunits each comprising the amino acid sequence of SEQ ID NO: 1; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, there is provided a pharmaceutical composition comprising (or consisting essentially of, or consisting of): a) about 1 mg/mL to about 100 mg/mL of a G-CSF dimer, wherein the G-CSF dimer comprises two monomeric subunits, and wherein each monomeric subunit comprises a G-CSF monomer (e.g., SEQ ID NO: 1), an Fc fragment (e.g., any of SEQ ID NOs: 2-5), and an optional linker (e.g., SEQ ID NO: 12) connecting the G-CSF monomer and the Fc fragment; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, there is provided a pharmaceutical composition comprising (or consisting essentially of, or consisting of): a) about 1 mg/mL to about 100 mg/mL of a G-CSF dimer, wherein the G-CSF dimer comprises two monomeric subunits each comprising (or consisting essentially of, or consisting of) the amino acid sequence of SEQ ID NO: 6; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, there is provided a pharmaceutical composition comprising (or consisting essentially of, or consisting of): a) about 1 mg/mL to about 100 mg/mL of a G-CSF dimer, wherein the G-CSF dimer is efbemalenograstim alfa; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, there is provided a pharmaceutical composition comprising (or consisting essentially of, or consisting of): a) about 1 mg/mL to about 100 mg/mL of a G-CSF dimer, wherein the G-CSF dimer comprises two monomeric subunits each comprising (or consisting essentially of, or consisting of) the amino acid sequence of SEQ ID NO: 7; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, there is provided a pharmaceutical composition comprising (or consisting essentially of, or consisting of): a) about 1 mg/mL to about 100 mg/mL of a G-CSF dimer, wherein the G-CSF dimer comprises two monomeric subunits each comprising (or consisting essentially of, or consisting of) the amino acid sequence of SEQ ID NO: 8; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, there is provided a pharmaceutical composition comprising (or consisting essentially of, or consisting of): a) about 1 mg/mL to about 100 mg/mL of a G-CSF dimer, wherein the G-CSF dimer comprises two monomeric subunits each comprising (or consisting essentially of, or consisting of) the amino acid sequence of SEQ ID NO: 9; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, there is provided a pharmaceutical composition comprising (or consisting essentially of, or consisting of): a) about 1 mg/mL to about 100 mg/mL of a G-CSF dimer, wherein the G-CSF dimer comprises two monomeric subunits each comprising (or consisting essentially of, or consisting of) the amino acid sequence of SEQ ID NO: 10; (b) about 1 mM to about 50 mM buffering agent; (c) about 0.1 mM to about 20 mM stabilizing agent; (d) about 1% (w/v) to about 15% (w/v) (e.g., about 1% (w/v) to about 10% (w/v)) tonicity agent; and (e) about 0.001% (w/v) to about 0.1% (w/v) surfactant. In some embodiments, the concentration of the G-CSF dimer is about 5 mg/mL to about 50 mg/mL, such as any of about 10 mg/mL, about 15 mg/mL, about 20 mg/mL, about 25 mg/mL, or about 30 mg/mL. In some embodiments, the concentration of the G-CSF dimer is about 18 mg/mL to about 22 mg/mL, e.g., about 20 mg/mL. In some embodiments, the buffering agent is sodium acetate. In some embodiments, the concentration of the sodium acetate is about 1 mM to about 30 mM, e.g., about 10 mM. In some embodiments, the stabilizing agent is EDTA. In some embodiments, the concentration of the EDTA is about 0.1 mM to about 10 mM, such as any of about 0.2 mM, about 0.5 mM, about 1 mM, about 1.5 mM, about 2 mM, or about 5 mM. In some embodiments, the concentration of the EDTA is about 1 mM. In some embodiments, the concentration of the EDTA is about 0.5 mM. In some embodiments, the tonicity agent is sorbitol. In some embodiments, the pharmaceutical composition comprises about 1% (w/v) to about 8% (w/v) sorbitol, such as any of about 1% (w/v), about 2% (w/v), about 3% (w/v), about 4% (w/v), about 5% (w/v), about 6% (w/v), about 7% (w/v), or about 8% (w/v) sorbitol. In some embodiments, the pharmaceutical composition comprises about 5% (w/v) sorbitol. In some embodiments, the tonicity agent is sucrose. In some embodiments, the pharmaceutical composition comprises about 5% (w/v) to about 15% (w/v) sucrose, e.g., about 9% (w/v) sucrose. In some embodiments, the surfactant is selected from group consisting of a polysorbate, a poloxamer, a polyoxyethelene alkyl ether, an alkyl phenyl polyoxyethylene ether, and a combination thereof. In some embodiments, the surfactant is a non-ionic surfactant. In some embodiments, the surfactant is polysorbate 20 (PS20) or polysorbate 80 (PS80). In some embodiments, the pharmaceutical composition comprises about 0.005% (w/v) to about 0.05% (w/v) PS20, such as any of about 0.006% (w/v), about 0.007% (w/v), about 0.008% (w/v), about 0.009% (w/v), about 0.01% (w/v), about 0.015% (w/v), about 0.02% (w/v), or about 0.025% (w/v) PS20. In some embodiments, the pharmaceutical composition comprises less than about 0.03% (w/v) PS20. In some embodiments, the pharmaceutical composition comprises about 0.01% (w/v) PS20. In some embodiments, the pharmaceutical composition has a pH of about 4.2 to about 6.2, such as about 4.8 to about 5.8, about 5.0 to about 5.4, or about 5.2. In some embodiments, the pharmaceutical composition has a pH of about 5.2. In some embodiments, the pharmaceutical composition is stable at 25±2° C. for at least about 1 month (e.g., 1, 2, or 3 months, or at least about 2 months). In some embodiments, the pharmaceutical composition is stable at 25±2° C. up to about 3 months. In some embodiments, the pharmaceutical composition has a purity of at least about 90% (e.g., at least about any of 91%, 92%, 93%, 94%, 95%, 99%, or higher) as measured by SE-HPLC. In some embodiments, the pharmaceutical composition comprises no more than about 2% aggregation. In some embodiments, the pharmaceutical composition has an osmolality of about 310 mOsm/kg. In some embodiments, the pharmaceutical composition comprises at least about 96% of the G-CSF dimer as measured by SE-HPLC following storage at about 2° C. to about 8° C. for at least about 1 month (e.g., at least about 2 months, such as about any of 6 months, 12 months, 24 months, 36 months, or longer). In some embodiments, the pharmaceutical composition comprises at least about 96% of the G-CSF dimer as measured by SE-HPLC following storage at about 2° C. to about 8° C. up to about 36 months. In some embodiments, the pharmaceutical composition comprises at least about 95.5% of the G-CSF dimer as measured by rCE-SDS following storage at about 2° C. to about 8° C. for at least about 1 month (e.g., at least about 2 months, such as about any of 6 months, 12 months, 24 months, 36 months, or longer). In some embodiments, the pharmaceutical composition comprises at least about 95.5% of the G-CSF dimer as measured by rCE-SDS following storage at about 2° C. to about 8° C. up to about 36 months. In some embodiments, the pharmaceutical composition comprises at least about 94% of the G-CSF dimer as measured by nrCE-SDS following storage at about 2° C. to about 8° C. for at least about 1 month (e.g., at least about 2 months, such as about any of 6 months, 12 months, 24 months, 36 months, or longer). In some embodiments, the pharmaceutical composition comprises at least about 94% of the G-CSF dimer as measured by nrCE-SDS following storage at about 2° C. to about 8° C. up to about 36 months. In some embodiments, the pharmaceutical composition comprises no more than about 1.5% of HMWS as measured by rCE-SDS following storage at about 2° C. to about 8° C. for at least about 1 month (e.g., at least about 2 months, such as about any of 6 months, 12 months, 24 months, 36 months, or longer). In some embodiments, the pharmaceutical composition comprises no more than about 1.5% of HMWS as measured by rCE-SDS following storage at about 2° C. to about 8° C. up to about 36 months. In some embodiments, the pharmaceutical composition comprises no more than about 1.5% of HMWS as measured by nrCE-SDS following storage at about 2° C. to about 8° C. for at least about 1 month (e.g., at least about 2 months, such as about any of 6 months, 12 months, 24 months, 36 months, or longer). In some embodiments, the pharmaceutical composition comprises no more than about 1.5% of HMWS as measured by nrCE-SDS following storage at about 2° C. to about 8° C. up to about 36 months. In some embodiments, the pharmaceutical composition comprises about 27.2% to about 39.0% acidic region, about 36.5% to about 60.1% main peak, and about 7.2% to about 30% basic region as measured by icIEF following storage at about 2° C. to about 8° C. for at least about 1 month (e.g., at least about 2 months, such as about any of 6 months, 12 months, 24 months, 36 months, or longer). In some embodiments, the pharmaceutical composition comprises about 27.2% to about 39.0% acidic region, about 36.5% to about 60.1% main peak, and about 7.2% to about 30% basic region as measured by icIEF following storage at about 2° C. to about 8° C. up to about 36 months. In some embodiments, the pharmaceutical composition retains at least about 70% (e.g., about 70% to about 130%) of its potency following storage at about 2° C. to about 8° C. for at least about 1 month (e.g., at least about 2 months, such as about any of 6 months, 12 months, 24 months, 36 months, or longer). In some embodiments, the pharmaceutical composition retains at least about 70% (e.g., about 70% to about 130%) of its potency following storage at about 2° C. to about 8° C. up to about 36 months. In some embodiments, the concentration of the pharmaceutical composition varies by less than about 2 mg/mL (e.g., less than about any of 1.5 mg/mL, 1 mg/mL, or 0.5 mg/mL) following storage at about 2° C. to about 8° C. for at least about 1 month (e.g., at least about 2 months, such as about any of 6 months, 12 months, 24 months, 36 months, or longer). In some embodiments, the concentration of the pharmaceutical composition varies by less than about 2 mg/mL (e.g., less than about any of 1.5 mg/mL, 1 mg/mL, or 0.5 mg/mL) following storage at about 2° C. to about 8° C. up to about 36 months.

In some embodiments, there is provided a pharmaceutical composition comprising (or consisting essentially of, or consisting of): a) about 18 mg/mL to about 22 mg/mL (e.g., about 20 mg/mL) of a G-CSF dimer, wherein the G-CSF dimer comprises two monomeric subunits each comprising the amino acid sequence of SEQ ID NO: 1; (b) about 10 mM sodium acetate; (c) about 1 mM EDTA; (d) about 5% (w/v) sorbitol; and (e) about 0.01% (w/v) PS20; wherein the pharmaceutical composition has a pH of about 5.2. In some embodiments, there is provided a pharmaceutical composition comprising (or consisting essentially of, or consisting of): a) about 18 mg/mL to about 22 mg/mL (e.g., about 20 mg/mL) of a G-CSF dimer, wherein the G-CSF dimer comprises two monomeric subunits, and wherein each monomeric subunit comprises a G-CSF monomer (e.g., SEQ ID NO: 1), an Fc fragment (e.g., any of SEQ ID NOs: 2-5), and an optional linker (e.g., SEQ ID NO: 12) connecting the G-CSF monomer and the Fc fragment; (b) about 10 mM sodium acetate; (c) about 1 mM EDTA; (d) about 5% (w/v) sorbitol; and (e) about 0.01% (w/v) PS20; wherein the pharmaceutical composition has a pH of about 5.2. In some embodiments, there is provided a pharmaceutical composition comprising (or consisting essentially of, or consisting of): a) about 18 mg/mL to about 22 mg/mL (e.g., about 20 mg/mL) of a G-CSF dimer, wherein the G-CSF dimer comprises two monomeric subunits each comprising (or consisting essentially of, or consisting of) the amino acid sequence of SEQ ID NO: 6; (b) about 10 mM sodium acetate; (c) about 1 mM EDTA; (d) about 5% (w/v) sorbitol; and (e) about 0.01% (w/v) PS20; wherein the pharmaceutical composition has a pH of about 5.2. In some embodiments, there is provided a pharmaceutical composition comprising (or consisting essentially of, or consisting of): a) about 18 mg/mL to about 22 mg/mL (e.g., about 20 mg/mL) of a G-CSF dimer, wherein the G-CSF dimer is efbemalenograstim alfa; (b) about 10 mM sodium acetate; (c) about 1 mM EDTA; (d) about 5% (w/v) sorbitol; and (e) about 0.01% (w/v) PS20; wherein the pharmaceutical composition has a pH of about 5.2. In some embodiments, there is provided a pharmaceutical composition comprising (or consisting essentially of, or consisting of): a) about 18 mg/mL to about 22 mg/mL (e.g., about 20 mg/mL) of a G-CSF dimer, wherein the G-CSF dimer comprises two monomeric subunits each comprising (or consisting essentially of, or consisting of) the amino acid sequence of SEQ ID NO: 7; (b) about 10 mM sodium acetate; (c) about 1 mM EDTA; (d) about 5% (w/v) sorbitol; and (e) about 0.01% (w/v) PS20; wherein the pharmaceutical composition has a pH of about 5.2. In some embodiments, there is provided a pharmaceutical composition comprising (or consisting essentially of, or consisting of): a) about 18 mg/mL to about 22 mg/mL (e.g., about 20 mg/mL) of a G-CSF dimer, wherein the G-CSF dimer comprises two monomeric subunits each comprising (or consisting essentially of, or consisting of) the amino acid sequence of SEQ ID NO: 8; (b) about 10 mM sodium acetate; (c) about 1 mM EDTA; (d) about 5% (w/v) sorbitol; and (e) about 0.01% (w/v) PS20; wherein the pharmaceutical composition has a pH of about 5.2. In some embodiments, there is provided a pharmaceutical composition comprising (or consisting essentially of, or consisting of): a) about 18 mg/mL to about 22 mg/mL (e.g., about 20 mg/mL) of a G-CSF dimer, wherein the G-CSF dimer comprises two monomeric subunits each comprising (or consisting essentially of, or consisting of) the amino acid sequence of SEQ ID NO: 9; (b) about 10 mM sodium acetate; (c) about 1 mM EDTA; (d) about 5% (w/v) sorbitol; and (e) about 0.01% (w/v) PS20; wherein the pharmaceutical composition has a pH of about 5.2. In some embodiments, there is provided a pharmaceutical composition comprising (or consisting essentially of, or consisting of): a) about 18 mg/mL to about 22 mg/mL (e.g., about 20 mg/mL) of a G-CSF dimer, wherein the G-CSF dimer comprises two monomeric subunits each comprising (or consisting essentially of, or consisting of) the amino acid sequence of SEQ ID NO: 10; (b) about 10 mM sodium acetate; (c) about 1 mM EDTA; (d) about 5% (w/v) sorbitol; and (e) about 0.01% (w/v) PS20; wherein the pharmaceutical composition has a pH of about 5.2. In some embodiments, the concentration of the G-CSF dimer is about 20 mg/mL. In some embodiments, the pharmaceutical composition has a pH of about 5.2. In some embodiments, the pharmaceutical composition is stable at 25±2° C. for at least about 1 month (e.g., 1, 2, or 3 months, or at least about 2 months). In some embodiments, the pharmaceutical composition is stable at 25±2° C. up to about 3 months. In some embodiments, the pharmaceutical composition has a purity of at least about 90% (e.g., at least about any of 91%, 92%, 93%, 94%, 95%, 99%, or higher) as measured by SE-HPLC. In some embodiments, the pharmaceutical composition comprises no more than about 2% aggregation. In some embodiments, the pharmaceutical composition has an osmolality of about 310 mOsm/kg. In some embodiments, the pharmaceutical composition comprises at least about 96% of the G-CSF dimer as measured by SE-HPLC following storage at about 2° C. to about 8° C. for at least about 1 month (e.g., at least about 2 months, such as about any of 6 months, 12 months, 24 months, 36 months, or longer). In some embodiments, the pharmaceutical composition comprises at least about 96% of the G-CSF dimer as measured by SE-HPLC following storage at about 2° C. to about 8° C. up to about 36 months. In some embodiments, the pharmaceutical composition comprises at least about 95.5% of the G-CSF dimer as measured by rCE-SDS following storage at about 2° C. to about 8° C. for at least about 1 month (e.g., at least about 2 months, such as about any of 6 months, 12 months, 24 months, 36 months, or longer). In some embodiments, the pharmaceutical composition comprises at least about 95.5% of the G-CSF dimer as measured by rCE-SDS following storage at about 2° C. to about 8° C. up to about 36 months. In some embodiments, the pharmaceutical composition comprises at least about 94% of the G-CSF dimer as measured by nrCE-SDS following storage at about 2° C. to about 8° C. for at least about 1 month (e.g., at least about 2 months, such as about any of 6 months, 12 months, 24 months, 36 months, or longer). In some embodiments, the pharmaceutical composition comprises at least about 94% of the G-CSF dimer as measured by nrCE-SDS following storage at about 2° C. to about 8° C. up to about 36 months. In some embodiments, the pharmaceutical composition comprises no more than about 1.5% of HMWS as measured by rCE-SDS following storage at about 2° C. to about 8° C. for at least about 1 month (e.g., at least about 2 months, such as about any of 6 months, 12 months, 24 months, 36 months, or longer). In some embodiments, the pharmaceutical composition comprises no more than about 1.5% of HMWS as measured by rCE-SDS following storage at about 2° C. to about 8° C. up to about 36 months. In some embodiments, the pharmaceutical composition comprises no more than about 1.5% of HMWS as measured by nrCE-SDS following storage at about 2° C. to about 8° C. for at least about 1 month (e.g., at least about 2 months, such as about any of 6 months, 12 months, 24 months, 36 months, or longer). In some embodiments, the pharmaceutical composition comprises no more than about 1.5% of HMWS as measured by nrCE-SDS following storage at about 2° C. to about 8° C. up to about 36 months. In some embodiments, the pharmaceutical composition comprises about 27.2% to about 39.0% acidic region, about 36.5% to about 60.1% main peak, and about 7.2% to about 30% basic region as measured by icIEF following storage at about 2° C. to about 8° C. for at least about 1 month (e.g., at least about 2 months, such as about any of 6 months, 12 months, 24 months, 36 months, or longer). In some embodiments, the pharmaceutical composition comprises about 27.2% to about 39.0% acidic region, about 36.5% to about 60.1% main peak, and about 7.2% to about 30% basic region as measured by icIEF following storage at about 2° C. to about 8° C. up to about 36 months. In some embodiments, the pharmaceutical composition retains at least about 70% (e.g., about 70% to about 130%) of its potency following storage at about 2° C. to about 8° C. for at least about 1 month (e.g., at least about 2 months, such as about any of 6 months, 12 months, 24 months, 36 months, or longer). In some embodiments, the pharmaceutical composition retains at least about 70% (e.g., about 70% to about 130%) of its potency following storage at about 2° C. to about 8° C. up to about 36 months. In some embodiments, the concentration of the pharmaceutical composition varies by less than about 2 mg/mL (e.g., less than about any of 1.5 mg/mL, 1 mg/mL, or 0.5 mg/mL) following storage at about 2° C. to about 8° C. for at least about 1 month (e.g., at least about 2 months, such as about any of 6 months, 12 months, 24 months, 36 months, or longer). In some embodiments, the concentration of the pharmaceutical composition varies by less than about 2 mg/mL (e.g., less than about any of 1.5 mg/mL, 1 mg/mL, or 0.5 mg/mL) following storage at about 2° C. to about 8° C. up to about 36 months.

### IX. Further Exemplary Embodiments

Embodiment 1. A pharmaceutical composition comprising: (a) about 1 mg/mL to about 100 mg/mL granulocyte colony-stimulating factor (G-CSF) dimer; (b) about 1 mM to about 50 mM buffering agent; (c) about 0.1 mM to about 20 mM stabilizing agent; (d) about 1% (w/v) to about 15% (w/v) tonicity agent; and about 0.001% (w/v) to about 0.1% (w/v) surfactant.

Embodiment 2. The pharmaceutical composition of Embodiment 1, wherein the G-CSF dimer comprises two monomeric subunits, and wherein each monomeric subunit comprises a G-CSF monomer and a dimerization domain.

Embodiment 3. The pharmaceutical composition of Embodiment 2, wherein the G-CSF monomer comprises the sequence of SEQ ID NO: 1.

Embodiment 4. The pharmaceutical composition of Embodiment 2 or 3, wherein the G-CSF monomer is connected to the dimerization domain via an optional linker.

Embodiment 5. The pharmaceutical composition of Embodiment 4, wherein the linker is about 6 to about 30 amino acids in length.

Embodiment 6. The pharmaceutical composition of Embodiment 4 or 5, wherein the linker comprises the sequence of SEQ ID NO: 12.

Embodiment 7. The pharmaceutical composition of any one of Embodiments 2-6, wherein the dimerization domain comprises at least two cysteines capable of forming intermolecular disulfide bonds.

Embodiment 8. The pharmaceutical composition of any one of Embodiments 2-7, wherein the dimerization domain comprises at least a portion of an Fc fragment.

Embodiment 9. The pharmaceutical composition of Embodiment 8, wherein the Fc fragment comprises CH2 and CH3 domains.

Embodiment 10. The pharmaceutical composition of Embodiment 8 or 9, wherein the Fc fragment is derived from IgG1 Fc, IgG2 Fc, IgG4 Fc, and fragments or variants thereof.

Embodiment 11. The pharmaceutical composition of any one of Embodiments 8-10, wherein the Fc fragment comprises an amino acid sequence selected from the group consisting of SEQ ID NOs: 2-5.

Embodiment 12. The pharmaceutical composition of any one of Embodiments 2-11, wherein the G-CSF monomer is N-terminal to the dimerization domain within each monomeric subunit.

Embodiment 13. The pharmaceutical composition of any one of Embodiments 2-12, wherein each monomeric subunit comprises an amino acid sequence having at least about 95% sequence identity to an amino acid sequence selected from the group consisting of SEQ ID NOs: 6-10.

Embodiment 14. The pharmaceutical composition of any one of Embodiments 2-13, wherein each monomeric subunit comprises an amino acid sequence selected from the group consisting of SEQ ID NOs: 6-10.

Embodiment 15. The pharmaceutical composition of any one of Embodiments 1-14, wherein the concentration of the G-CSF dimer is about 5 mg/mL to about 50 mg/mL.

Embodiment 16. The pharmaceutical composition of any one of Embodiments 1-15, wherein the concentration of the G-CSF dimer is about 20 mg/mL.

Embodiment 17. The pharmaceutical composition of any one of Embodiments 1-16, wherein the buffering agent is sodium acetate.

Embodiment 18. The pharmaceutical composition of Embodiment 17, wherein the concentration of the sodium acetate is about 1 mM to about 30 mM.

Embodiment 19. The pharmaceutical composition of Embodiment 17 or 18, wherein the concentration of the sodium acetate is about 10 mM.

Embodiment 20. The pharmaceutical composition of any one of Embodiments 1-19, wherein the stabilizing agent is EDTA.

Embodiment 21. The pharmaceutical composition of Embodiment 20, wherein the concentration of the EDTA is about 0.1 mM to about 10 mM.

Embodiment 22. The pharmaceutical composition of Embodiment 20 or 21, wherein the concentration of the EDTA is about 1 mM.

Embodiment 23. The pharmaceutical composition of any one of Embodiments 1-22, wherein the tonicity agent is sorbitol.

Embodiment 24. The pharmaceutical composition of Embodiment 23, wherein said pharmaceutical composition comprises about 1% (w/v) to about 8% (w/v) sorbitol.

Embodiment 25. The pharmaceutical composition of Embodiment 23 or 24, wherein said pharmaceutical composition comprises about 5% (w/v) sorbitol.

Embodiment 26. The pharmaceutical composition of any one of Embodiments 1-22, wherein the tonicity agent is sucrose.

Embodiment 27. The pharmaceutical composition of Embodiment 26, wherein said pharmaceutical composition comprises about 5% (w/v) to about 15% (w/v) sucrose.

Embodiment 28. The pharmaceutical composition of Embodiment 26 or 27, wherein said pharmaceutical composition comprises about 9% (w/v) sucrose.

Embodiment 29. The pharmaceutical composition of any one of Embodiments 1-28, wherein the surfactant is selected from group consisting of a polysorbate, a poloxamer, a polyoxyethelene alkyl ether, an alkyl phenyl polyoxyethylene ether, and a combination thereof.

Embodiment 30. The pharmaceutical composition of any one of Embodiments 1-29, wherein the surfactant is polysorbate 20 (PS20) or polysorbate 80 (PS80).

Embodiment 31. The pharmaceutical composition of Embodiment 30, wherein said pharmaceutical composition comprises about 0.005% (w/v) to about 0.05% (w/v) PS20.

Embodiment 32. The pharmaceutical composition of Embodiment 30 or 31, wherein said pharmaceutical composition comprises about 0.01% (w/v) PS20.

Embodiment 33. The pharmaceutical composition of any one of Embodiments 1-32, wherein the pH of the pharmaceutical composition is about 4.2 to about 6.2.

Embodiment 34. The pharmaceutical composition of any one of Embodiments 1-33, wherein the pH of the pharmaceutical composition is about 4.8 to about 5.8.

Embodiment 35. The pharmaceutical composition of any one of Embodiments 1-34, wherein the pH of the pharmaceutical composition is about 5.0 to about 5.4.

Embodiment 36. The pharmaceutical composition of any one of Embodiments 1-35, wherein the pH of the pharmaceutical composition is about 5.2.

Embodiment 37. The pharmaceutical composition of any one of Embodiments 1-25 and 29-36, comprising: (a) about 20 mg/mL of the G-CSF dimer; (b) about 10 mM sodium acetate; (c) about 1 mM IA; (d) about 5% (w/v) sorbitol; and (e) about 0.01% (w/v) PS20, wherein the pharmaceutical composition has a pH of about 5.2.

Embodiment 38. The pharmaceutical composition of any one of Embodiments 1-37, wherein the G-CSF dimer comprises two monomeric subunits comprising an amino acid sequence of SEQ ID NO: 6.

Embodiment 39. The pharmaceutical composition of any one of Embodiments 1-38, wherein the pharmaceutical composition is stable at 25±2° C. for at least about 1 month.

Embodiment 40. The pharmaceutical composition of any one of Embodiments 1-39, wherein the pharmaceutical composition is stable at 25±2° C. for at least about 2 months.

Embodiment 41. The pharmaceutical composition of any one of Embodiments 1-40, wherein the pharmaceutical composition has a purity of G-CSF dimer of at least about 90% as assessed by SE-HPLC.

Embodiment 42. The pharmaceutical composition of any one of Embodiments 1-41, wherein the pharmaceutical composition has an osmolality of about 268-360 mOsm/kg.

Embodiment 43. The pharmaceutical composition of any one of Embodiments 1-42, wherein the pharmaceutical composition comprises main peak at least about 85% in relation to the total area of all peaks when measured by SE-HPLC after at least about 1 month of storage under about 25±2° C.

Embodiment 44. The pharmaceutical composition of any one of Embodiments 1-43, wherein the pharmaceutical composition comprises main peak at least about 85% in relation to the total area of all peaks when measured by SE-HPLC after at least about 2 months of storage under about 25±2° C.

Embodiment 45. The pharmaceutical composition of any one of Embodiments 1-44, wherein the pharmaceutical composition comprises dimer less than about 2.5% in relation to the total area of all peaks when measured by SE-HPLC after at least about 1 month of storage under about 25±2° C.

Embodiment 46. The pharmaceutical composition of any one of Embodiments 1-45, wherein the pharmaceutical composition comprises dimer less than about 2.5% in relation to the total area of all peaks when measured by SE-HPLC after at least about 2 months of storage under about 25±2° C.

Embodiment 47. The pharmaceutical composition of any one of Embodiments 1-46, wherein the pharmaceutical composition comprises an aggregate in no more than about 2% in relation to the total area of all peaks when measured by SE-HPLC after at least about 1 month of storage under about 25±2° C.

Embodiment 48. The pharmaceutical composition of any one of Embodiments 1-47, wherein the pharmaceutical composition comprises an aggregate in no more than about 2% in relation to the total area of all peaks when measured by SE-HPLC after at least about 2 months of storage under about 25±2° C.

Embodiment 49. The pharmaceutical composition of any one of Embodiments 1-48, wherein the pharmaceutical composition comprises main peak at least about 85% in relation to the total area of all peaks when measured by SE-HPLC after 36 months of storage under about 2° C. to about 8° C.

Embodiment 50. The pharmaceutical composition of any one of Embodiments 1-49, wherein the pharmaceutical composition comprises at least 96% of the G-CSF dimer by rCE-SDS after 1 month of storage under 25±2° C.

Embodiment 51. The pharmaceutical composition of any one of Embodiments 1-50, wherein the pharmaceutical composition comprises at least 96% of the G-CSF dimer by rCE-SDS after 2 months of storage under 25±2° C.

Embodiment 52. The pharmaceutical composition of any one of Embodiments 1-51, wherein the pharmaceutical composition comprises at least 95.5% of the G-CSF dimer by rCE-SDS after 36 months of storage under 2-8° C.

Embodiment 53. The pharmaceutical composition of any one of Embodiments 1-52, wherein the pharmaceutical composition comprises no more than 1.5% high molecular weight species by rCE-SDS after 1 month of storage under 25±2° C.

Embodiment 54. The pharmaceutical composition of any one of Embodiments 1-53, wherein the pharmaceutical composition comprises no more than 1.5% high molecular weight species by rCE-SDS after 2 months of storage under 25±2° C.

Embodiment 55. The pharmaceutical composition of any one of Embodiments 1-54, wherein the pharmaceutical composition comprises no more than 1.5% high molecular weight species by rCE-SDS after 36 months of storage under 2-8° C.

Embodiment 56. The pharmaceutical composition of any one of Embodiments 1-55, wherein the pharmaceutical composition comprises at least 94% of the G-CSF dimer by nrCE-SDS after 1 month of storage under 25±2° C.

Embodiment 57. The pharmaceutical composition of any one of Embodiments 1-56, wherein the pharmaceutical composition comprises at least 94% of the G-CSF dimer by nrCE-SDS after 2 months of storage under 25±2° C.

Embodiment 58. The pharmaceutical composition of any one of Embodiments 1-57, wherein the pharmaceutical composition comprises at least 94% of G-CSF dimer by nrCE-SDS after 36 months of storage under 2-8° C.

Embodiment 59. The pharmaceutical composition of any one of Embodiments 1-58, wherein the pharmaceutical composition comprises no more than 1.5% high molecular weight species by nrCE-SDS after 1 month of storage under 25±2° C.

Embodiment 60. The pharmaceutical composition of any one of Embodiments 1-59, wherein the pharmaceutical composition comprises no more than 1.5% high molecular weight species by nrCE-SDS after 2 months of storage under 25±2° C.

Embodiment 61. The pharmaceutical composition of any one of Embodiments 1-60, wherein the pharmaceutical composition comprises no more than 1.5% high molecular weight species by nrCE-SDS after 36 months of storage under 2-8° C.

Embodiment 62. The pharmaceutical composition of any one of Embodiments 1-61, wherein the pharmaceutical composition comprises about 27.2% to about 39.0% acidic region, about 36.5% to about 60.1% main peak, and about 7.2% to about 30% basic region as measured by icIEF after 1 month of storage under 25±2° C.

Embodiment 63. The pharmaceutical composition of any one of Embodiments 1-62, wherein the pharmaceutical composition comprises about 27.2% to about 39.0% acidic region, about 36.5% to about 60.1% main peak, and about 7.2% to about 30% basic region as measured by icIEF after 2 months of storage under 25±2° C.

Embodiment 64. The pharmaceutical composition of any one of Embodiments 1-63, wherein the pharmaceutical composition comprises about 27.2% to about 39.0% acidic region, about 36.5% to about 60.1% main peak, and about 7.2% to about 30% basic region as measured by icIEF after 36 months of storage under 2-8° C.

Embodiment 65. The pharmaceutical composition of any one of Embodiments 1-64, wherein the pharmaceutical composition retains at least 70% potency after 1 month of storage under 25±2° C.

Embodiment 66. The pharmaceutical composition of any one of Embodiments 1-65, wherein the pharmaceutical composition retains at least 70% potency after 2 months of storage under 25±2° C.

Embodiment 67. The pharmaceutical composition of any one of Embodiments 1-66, wherein the pharmaceutical composition retains at least 70% potency after 36 months of storage under 2-8° C.

Embodiment 68. The pharmaceutical composition of any one of Embodiments 1-67, wherein the concentration of the pharmaceutical composition varies by less than 0.5 mg/mL after 1 month of storage under 25±2° C.

Embodiment 69. The pharmaceutical composition of any one of Embodiments 1-68, wherein the concentration of the pharmaceutical composition varies by less than 0.5 mg/mL after 2 months of storage under 25±2° C.

Embodiment 70. The pharmaceutical composition of any one of Embodiments 1-69, wherein the concentration of pharmaceutical composition varies by less than 0.5 mg/mL after 36 months of storage under 2-8° C.

Embodiment 71. A syringe comprising the pharmaceutical composition of any one of Embodiments 1-70.

Embodiment 72. The syringe of Embodiment 71, wherein the syringe is for single use.

Embodiment 73. The syringe of Embodiment 71 or 72, wherein the volume of the liquid pharmaceutical composition within the syringe is about 1 mL.

Embodiment 74. The syringe of any one of Embodiments 71-73, wherein the syringe is sterile.

Embodiment 75. A method of treating a disease or condition in an individual, comprising administering to the individual an effective amount of the liquid pharmaceutical composition of any one of Embodiments 1-70.

Embodiment 76. The method of Embodiment 75, wherein the pharmaceutical composition is administered at a dosage of from about 0.01 mg/kg to about 1 mg/kg.

Embodiment 77. The method of Embodiment 75 or 76, wherein the pharmaceutical composition is administered once every four weeks.

Embodiment 78. The method of any one of Embodiments 75-77, wherein the pharmaceutical composition is administered subcutaneously.

Embodiment 79. The method of any one of Embodiments 75-78, wherein the disease or condition is selected from the group consisting of neutropenia, stroke, spinal injury, neurological disorders accompanied with blood brain barrier injury, Parkinson's disease, Alzheimer's disease, Huntington disease, amyotrophic lateral sclerosis, spinal muscular atrophy, primary lateral sclerosis, spinal cerebellar ataxias, and mobilization of hematopoietic stem cells into peripheral blood in allogeneic blood stem cell transplantation.

Embodiment 80. The method of any one of Embodiments 75-79, wherein the disease or condition is neutropenia.

Embodiment 81. The method of Embodiment 80, wherein the neutropenia is induced by chemotherapy and/or radiotherapy.

Embodiment 82. The method of Embodiment 81, wherein the pharmaceutical composition is administered at about 20 mg once per chemotherapy cycle.

Embodiment 83. A method of manufacturing the pharmaceutical composition of any one of Embodiments 1-70, comprising: (i) culturing a host cell under a condition suitable for expressing the G-CSF dimer; (ii) isolating the expressed G-CSF dimer from the cell culture; (iii) purifying the expressed G-CSF dimer; and (iv) formulating the purified G-CSF dimer with the buffering agent, the stabilizing agent, the tonicity agent, and the surfactant.

Embodiment 84. The method of Embodiment 83, wherein the purification step comprises one or more of: affinity chromatography, viral inactivation, ion exchange chromatography, mixed-mode chromatography, and filtration.

### EXAMPLES

The following example is included for illustrative purposes only and is not intended to limit the scope of the invention.

### Example 1. Preparation of Exemplary G-CSF-Fc Dimer

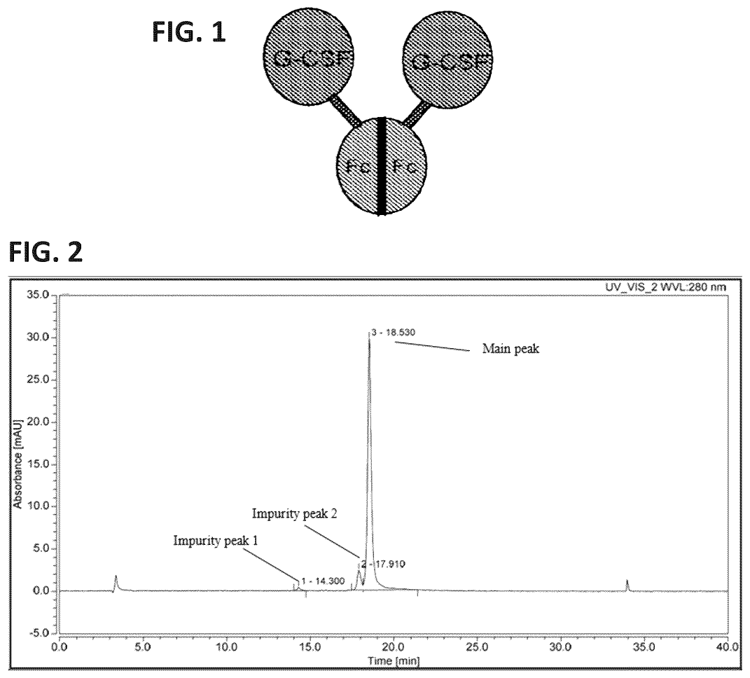

An exemplary G-CSF dimer is a homodimer of two G-CSF-Fc monomeric subunits, each comprising from the N-terminus to the C-terminus: a human G-CSF monomer (SEQ ID NO: 1), a GS linker (SEQ ID NO: 12), and a human IgG2-derived Fc fragment (SEQ ID NO: 2), wherein each monomeric subunit comprises the amino acid sequence of SEQ ID NO: 6. The two monomeric subunits are connected by two pairs of disulfide bonds. This exemplary G-CSF dimer is herein also referred to as G-CSF-Fc dimer F-627 or efbemalenograstim alfa, exemplified in FIG. 1. The hIgG2-derived Fc fragment has a P297S substitution when numbered from the N-terminus of the entire polypeptide chain of the G-CSF-Fc monomeric subunit, which corresponds to a P331S substitution located in the CH2 domain according to EU numbering. Each G-CSF-Fc monomeric subunit has one N-linked glycosylation site at N263 when numbered from the N-terminus of the entire polypeptide chain of the G-CSF-Fc monomeric subunit, which corresponds to N297 located in the CH2 domain according to EU numbering. Each G-CSF-Fc monomeric subunit also has one 0-linked glycosylation site at T133 in the hG-CSF monomer. Efbemalenograstim alfa has a predicted molecular weight of about 89,497 Da (calculated based on the entire amino acid sequence, without carbohydrates).

Efbemalenograstim alfa was constructed and produced in Chinese hamster ovary cells by the methods essentially as described in U.S. Pat. No. 8,557,546B2, the content of which is incorporated herein by reference in its entirety. The intact molecule of efbemalenograstim alfa has a molecular weight of about 93.4 KD.

The following examples were carried out with the purified G-CSF-Fc dimer efbemalenograstim alfa.

### Example 2. Liquid Formulation in Prefilled Syringe Screen 1

Formulation development studies were conducted for the purpose of developing a high concentration, liquid formulation of G-CSF-Fc dimer that could be used in prefilled syringe (PFS) for subcutaneous injection. Purified G-CSF-Fc dimer efbemalenograstim alfa in 10 mM phosphate buffer pH 7.2 in bulk was used as starting material. The bulk G-CSF-Fc dimer was concentrated, and dialyzed against formulation buffers. After dialysis, the protein concentration was determined by A280. Polysorbate 20 (PS20) was spiked into each formulated sample to the target concentration.

In order to evaluate the effect of pH on the stability of G-CSF-Fc dimer, a formulation study was set up at pH 6.0-8.0 in 10 mM sodium phosphate as well as at pH 5.0 in 10 mM sodium acetate. The G-CSF-Fc dimer was formulated in four formulation buffers (Table 2). The G-CSF-Fc dimer bulk was dialyzed and diluted to a final concentration of 10 mg/mL. Then the formulation samples were sterilized by using 0.2 μm syringe filter and vialed into pre-filled syringes in a particle-free and sterile hood. The samples were stored at 25±2° C. for one week and analyzed using size exclusion high-performance liquid chromatography (SE-HPLC).

TABLE 2
Buffers in the formulation

| Sample | | | Tonicity | |
|---|---|---|---|---|
| No. | pH | Buffering agent | agent | Surfactant |
| 1 | 5.0 | 10 mM sodium acetate | 5% sorbitol | 0.004% PS20 |
| 2 | 6.0 | 10 mM sodium phosphate | 5% sorbitol | 0.004% PS20 |
| 3 | 7.0 | 10 mM sodium phosphate | 5% sorbitol | 0.004% PS20 |
| 4 | 8.0 | 10 mM sodium phosphate | 5% sorbitol | 0.004% PS20 |

### Size Exclusion High-Performance Liquid Chromatography (SE-HPLC)

SE-HPLC was established to determine the degree of aggregation of the protein. SE-HPLC was performed as follows. The mobile phase contained 0.05 M acetate salt and 0.3 M sodium sulfate at pH 5.0. A protection column of TSK Gel G3000 SWxl 6.0×40 mm and a chromatographic column TSK Gel G3000 SWxl 7.80×300 mm were used. Samples were diluted with the mobile phase to 4 mg/mL. The preparation buffer was diluted in the same way as above and used as blank solution. 25 μL of the blank solution and the test solution were injected into the liquid chromatograph to start the detection. The flow rate was 0.6 mL/min, the acquisition time was 27 minutes, and the column temperature was 25±2° C. The detection was performed at 280 nm. The data was analyzed using Chem Station software.

TABLE 3
Stability of G-CSF-Fc dimer in different formulation buffers

| | |
|---|---|
| | SE-HPLC |
| Sample | % Mean Peak |

| No. | 0 w | 1 w |
|---|---|---|
| 1 | 98.1 | 98.9 |
| 2 | 97.3 | 97.6 |
| 3 | 96.8 | 95.4 |
| 4 | 96.6 | 93.5 |

*0 w: 0 week; 1 w: 1 week

The results are shown in Table 3. After storage at 25±2° C. for one week, there was a decreasing trend in percent main peak of G-CSF-Fc dimer as measured by SE-HPLC, as the pH of the formulation buffers increased. The results of SE-HPLC show that G-CSF-Fc dimer exhibits the best stability profile when formulated in 10 mM sodium acetate, 5% (w/v) sorbitol, 0.004% (w/v) PS20, pH 5.0.

### Example 3. Liquid Formulation in Prefilled Syringe Screen 2

To evaluate the effect of different pH on the stability of G-CSF-Fc dimer, a study was performed to assess the G-CSF-Fc dimer efbemalenograstim alfa formulated in formulation buffers containing 10 mM acetate, 1 mM EDTA, 0.01% (w/v) PS20 and two different tonicity agents (Table 4). G-CSF-Fc dimer bulk was dialyzed and diluted to a final concentration of 15 mg/mL using the four formulation buffers. Then the formulation samples were sterilized by using 0.2 μm syringe filter and vialed into pre-filled syringes in a particle-free and sterile hood. The formulated samples were stored at 25±2° C. for up to 12 weeks and analyzed using reduced sodium dodecyl sulfate-polyacrylamide gel electrophoresis (rSDS-PAGE), non-reduced SDS-PAGE (nrSDS-PAGE), SE-HPLC and reverse phase high-performance liquid chromatography (RP-HPLC).

TABLE 4
Buffers in the formulation

| Sample | | Buffering | Tonicity | Stabilizing | |
|---|---|---|---|---|---|
| No. | pH | agent | agent | agent | Surfactant |
| 1 | 4.8 | 10 mM acetate | 5% sorbitol | 1 mM EDTA | 0.01% PS20 |
| 2 | 5.2 | 10 mM acetate | 5% sorbitol | 1 mM EDTA | 0.01% PS20 |
| 3 | 4.8 | 10 mM acetate | 9% sucrose | 1 mM EDTA | 0.01% PS20 |
| 4 | 5.2 | 10 mM acetate | 9% sucrose | 1 mM EDTA | 0.01% PS20 |

Reducing and non-reducing SDS-PAGE were performed for the detection of degradation products (fragments) and aggregates of the protein.

Reduced Sodium Dodecyl Sulfate-Polyacrylamide Gel Electrophoresis (rSDS-PAGE)

The rSDS-PAGE was performed as follows. The samples were diluted to about 2 mg/mL, and mixed with loading buffer (Invitrogen, Cat #NP0007), sample reducing agent (Invitrogen, Cat #NP0004), 100 mM N-Ethylmaleimide (NEM) reagent. Then the mixed samples were electrophoresed on 4-12% Bis-Tris gels (Invitrogen, Cat #NP0321BOX) at 200 V for 35 min. Equal amount of protein (10 g) was loaded for each sample. The gels were then stained with staining solution (Bio-Rad, Cat #161-0436) for 1 h and distained in distaining solution (Bio-Rad, Cat #161-0438). The gels were scanned using Canoscan Lide 200. The images were analyzed using Quantity One Software.

Non-Reduced SDS-PAGE (nrSDS-PAGE)

The nrSDS-PAGE was performed as follows. The samples were diluted to about 2 mg/mL, and mixed with loading buffer (Invitrogen, Cat #NP0007), 100 mM N-Ethylmaleimide (NEM) reagent. Then the mixed samples were electrophoresed on 4-12% Bis-Tris gels (Invitrogen, Cat #NP0321BOX) at 200 V for 35 min. Equal amount of protein (10 g) was loaded for each sample. The gels were then stained with staining solution (Bio-Rad, Cat #161-0436) for 1 h and distained in distaining solution (Bio-Rad, Cat #161-0438). The gels were scanned using Canoscan Lide 200. The images were analyzed using Quantity One Software.

### Reverse Phase High-Performance Liquid Chromatography (RP-HPLC)

RP-HPLC was established to determine the change of polarity of the protein. RP-HPLC was performed on Agilent 1200 HPLC using a reverse phase column (Waters, Cat #186000289), with mobile phase A being H₂O+0.1% trifluoroacetate (DikmaPure, Cat #50134) and mobile phase B being acetonitrile+0.1% trifluoroacetate (TEDIA, Cat #AS1122-001). Mobile phases A and B were mixed and de-gassed using sonification. The parameters for the RP-HPLC are listed in Table 5.

TABLE 5
RP-HPLC parameters

| | | |
|---|---|---|
| Flow rate | 1.0 | mL/min |
| Run time | 40 | min |
| Sample injection temperature | 4° | C. |
| Column temperature | 30° | C. |
| Detection wavelength | 280 | nm |
| Injection amount | 20 | μg |
| Injection volume | 20 | μL |
| Maximum column pressure | 250 | bar |

Samples were diluted to 1.0 mg/mL with respective sample buffer, and the sample buffer was used as a control. The column was equilibrated with mobile phase A at an initial flow rate of 0.5 mL/min. The flow rate was adjusted to 1.0 mL/min after 30 minutes. After the baseline stabilized (less than 0.5 mAu/60 min), the sample analysis was performed.

The results of rSDS-PAGE, nrSDS-PAGE, SE-HPLC and RP-HPLC analysis for the samples after stored at 25±2° C. for up to 12 weeks are shown in Table 6. An exemplary chromatogram of G-CSF-Fc dimer (Sample No. 2) at week 0 by RP-HPLC is shown in FIG. 2.

TABLE 6
Stability of G-CSF-Fc dimer in different formulation buffers

| | nr-SDS-PAGE | | | | r-SDS-PAGE | | | | SE-HPLC | | | | RP-HPLC | | | |
|---|---|---|---|---|---|---|---|---|---|---|---|---|---|---|---|---|
| Sample | % Main Band | | | | % Main Band | | | | % Main Peak | | | | % Main Peak | | | |
| No. | 0 w | 4 w | 8 W | 12 w | 0 w | 4 w | 8 w | 12 w | 0 w | 4 w | 8 w | 12 w | 0 w | 4 w | 8 w | 12 w |
| 1 | 88.7 | 78.5 | 75.0 | 70.3 | 95.3 | 89.7 | 85.0 | 85.2 | 99.4 | 99.6 | 99.8 | 99.3 | 96.6 | 94.2 | 91.4 | 89.0 |
| 2 | 90.1 | 82.8 | 79.9 | 77.9 | 95.7 | 93.3 | 88.4 | 87.4 | 99.5 | 99.1 | 99.4 | 98.9 | 97.0 | 95.3 | 94.1 | 92.3 |
| 3 | 90.7 | 80.3 | 74.4 | 69.6 | 95.4 | 91.5 | 85.9 | 82.6 | 99.5 | 99.6 | 99.6 | 99.3 | 96.7 | 94.0 | 91.5 | 88.7 |
| 4 | 89.1 | 83.6 | 81.0 | 75.3 | 96.5 | 93.1 | 88.7 | 87.9 | 99.4 | 99.3 | 99.2 | 99.0 | 97.0 | 95.3 | 93.8 | 92.3 |

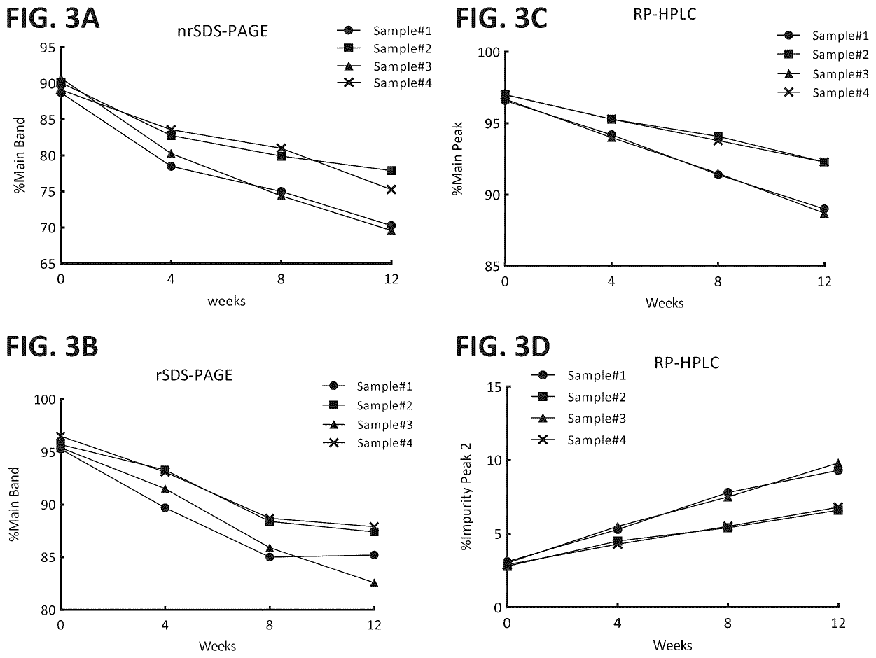

As shown in FIGS. 3A-3B, the decrease in percent main band of G-CSF-Fc dimer by SDS-PAGE in Sample No. 1 (10 mM sodium acetate, 5% (w/v) sorbitol, 0.01% (w/v) PS 20, 1 mM EDTA, pH 4.8) was higher in comparison to that of Sample No. 2 (pH 5.2, formulation composition and concentration was the same as Sample No. 1). As shown in FIG. 3C, the percent main peak of G-CSF-Fc dimer by RP-HPLC in Sample No. 1 decreased faster in comparison to that of Sample No. 2. Similarly, the decrease in percent main band of G-CSF-Fc dimer by SDS-PAGE in Sample No. 3 (10 mM sodium acetate, 9% (w/v) sucrose, 0.01% (w/v) PS 20, 1 mM EDTA, pH 4.8) was higher in comparison to that of Sample No. 4 (pH 5.2, formulation composition and concentration was the same as Sample No. 3). The percent main peak of G-CSF-Fc dimer by RP-HPLC in Sample No. 3 decreased faster in comparison to that of Sample No. 4 (FIG. 3C). There was no significant change observed by SE-HPLC. The percent impurity peak 2 of G-CSF-Fc dimer by RP-HPLC in Sample No. 3 increased faster in comparison to that of Sample No. 4 (FIG. 3D). These results unexpectedly suggest that the G-CSF-Fc dimer is unstable at low pH (e.g., pH 4.8), but is more stable around pH 5.2.

### Example 4. Liquid Formulation in Prefilled Syringe Screen 3

Based on the results of screen study 1 and 2, a further screen was initiated with a target of pH 5.2 in buffers to establish the acceptable pH range for G-CSF-Fc dimer protein. The G-CSF-Fc dimer efbemalenograstim alfa was formulated in six formulation buffers (Table 7). G-CSF-Fc dimer bulk was dialyzed and diluted to a final concentration of 20 mg/mL using the six formulation buffers. Then the formulation samples were sterilized by using 0.2 μm syringe filter, and vialed into pre-filled syringes in a particle-free and sterile hood. The formulated samples were stored at 25±2° C. for up to 12 weeks and analyzed using rSDS-PAGE, nrSDS-PAGE, SE-HPLC and RP-HPLC.

TABLE 7
Buffers of the formulation

| Sample | | Buffering | Tonicity | Stabilizing | |
|---|---|---|---|---|---|
| No. | pH | agent | agent | agent | Surfactant |
| 1 | 5.0 | 10 mM Acetate | 5% Sorbitol | 1 mM EDTA | 0.01% PS20 |
| 2 | 5.2 | 10 mM Acetate | 5% Sorbitol | 1 mM EDTA | 0.01% PS20 |
| 3 | 5.4 | 10 mM Acetate | 5% Sorbitol | 1 mM EDTA | 0.01% PS20 |
| 4 | 5.0 | 10 mM Acetate | 9% Sucrose | 1 mM EDTA | 0.01% PS20 |
| 5 | 5.2 | 10 mM Acetate | 9% Sucrose | 1 mM EDTA | 0.01% PS20 |
| 6 | 5.4 | 10 mM Acetate | 9% Sucrose | 1 mM EDTA | 0.01% PS20 |

The results of rCE-SDS-PAGE, nrCE-SDS-PAGE, SE-HPLC and RP-HPLC analysis for the samples after storage at 25±2° C. for up to 12 weeks are shown in Table 8.

TABLE 8
Stability of G-CSF-Fc dimer in different formulation buffers

| | nr-SDS-PAGE | | | | r-SDS-PAGE | | | | SE-HPLC | | | | RP-HPLC | | | |
|---|---|---|---|---|---|---|---|---|---|---|---|---|---|---|---|---|
| Sample | % Main Band | | | | % Main Band | | | | % Main Peak | | | | % Main Peak | | | |
| No. | 0 w | 4 w | 8 w | 12 w | 0 w | 4 w | 8 w | 12 w | 0 w | 4 w | 8 w | 12 w | 0 w | 4 w | 8 w | 12 w |
| 1 | 88.9 | 84.5 | 72.4 | 70.2 | 95.8 | 89.6 | 84.7 | 85.0 | 96.0 | 99 | 99.2 | 99.3 | 97.8 | 94.6 | 91.1 | 89.0 |
| 2 | 89.7 | 87.4 | 76.5 | 72.5 | 96 | 90.9 | 85.8 | 87.0 | 95.6 | 98.8 | 99.1 | 99.2 | 97.6 | 95.1 | 92.0 | 90.4 |
| 3 | 88.9 | 87.8 | 78.9 | 75.0 | 95.7 | 90.2 | 88.0 | 89.0 | 95.8 | 98.5 | 98.6 | 98.8 | 97.5 | 95.6 | 93.0 | 91.8 |
| 4 | 90.2 | 86.6 | 75.8 | 70.5 | 95.5 | 88.7 | 85.4 | 85.9 | 95.9 | 99.2 | 99.3 | 99.3 | 97.6 | 94.7 | 91.2 | 88.5 |
| 5 | 90.2 | 87.8 | 80.2 | 74.4 | 95.2 | 89.7 | 86.5 | 87.0 | 95.6 | 99 | 99.0 | 99.2 | 97.6 | 95.1 | 92.4 | 90.6 |
| 6 | 90.3 | 90.1 | 83.3 | 76.2 | 96.4 | 88.6 | 88.2 | 88.3 | 95.9 | 98.6 | 98.6 | 99.0 | 97.6 | 95.7 | 93.3 | 91.9 |

After storage at 25±2° C. for 12 weeks, the results of SDS-PAGE and RP-HPLC showed there was a slight decrease trend in the reduction of % main band when pH increased from 5.0 to 5.4 for both types of formulations. There was no significant change observed by SE-HPLC. Since SDS-PAGE data have larger experimental variability than RP-HPLC and SEC-HPLC, the observed differences are not considering as significant. The results indicate that G-CSF dimer is stable when formulated at 20 mg/mL in a pharmaceutical formulation containing 10 mM acetate, 1 mM EDTA, 0.01% (w/v) PS20, either tonicity agents (i.e., 5% sorbitol or 9% sucrose) at pH range of 5.0 to 5.4. This would support the manufacturing specification of pH in the range of 5.0 to 5.4.

### Example 5. Liquid Formulation in Prefilled Syringe Screen 4

In order to evaluate the effect of combination of surfactant and stabilizing agent on the stability of G-CSF-Fc dimer, a study was performed for the G-CSF-Fc dimer efbemalenograstim alfa at 20 mg/mL in six formulation buffers at pH 5.2 with different concentrations of PS20 and EDTA (Table 9). G-CSF-Fc dimer bulk was dialyzed and diluted to a final concentration of 20 mg/mL using the six formulation buffers. Then the formulation samples were sterilized by using 0.2 μm syringe filter, and vialed into pre-filled syringes in a particle-free and sterile hood. The formulation samples were stored at 25±2° C. for up to 12 weeks, and analyzed using rSDS-PAGE, nrSDS-PAGE, SE-HPLC and RP-HPLC.

TABLE 9
Composition of formulation

| Sam- | | | | | |
|---|---|---|---|---|---|
| ple | | Buffering | Tonicity | Stabilizing | |
| No. | pH | agent | agent | agent | Surfactant |
| 1 | 5.2 | 10 mM Acetate | 5% Sorbitol | 0.5 mM EDTA | 0.01% PS20 |
| 2 | 5.2 | 10 mM Acetate | 5% Sorbitol | 1 mM EDTA | 0.01% PS20 |
| 3 | 5.2 | 10 mM Acetate | 5% Sorbitol | 0.5 mM EDTA | 0.03% PS20 |
| 4 | 5.2 | 10 mM Acetate | 9% Sucrose | 0.5 mM EDTA | 0.01% PS20 |
| 5 | 5.2 | 10 mM Acetate | 9% Sucrose | 1 mM EDTA | 0.01% PS20 |
| 6 | 5.2 | 10 mM Acetate | 9% Sucrose | 0.5 mM EDTA | 0.03% PS20 |

The results of rCE-SDS-PAGE, nrCE-SDS-PAGE, SE-HPLC and RP-HPLC analysis for the formulation samples are shown in Table 10.

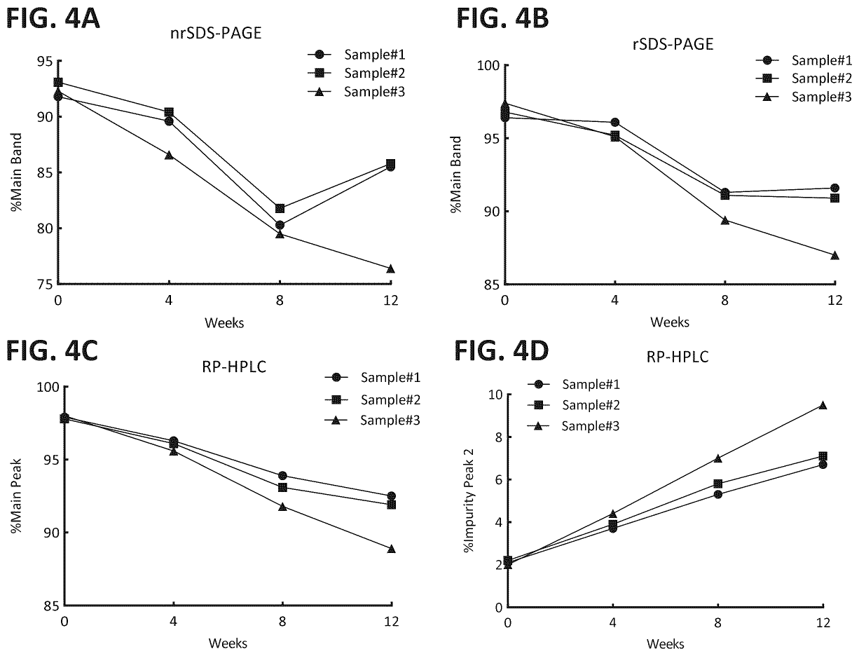

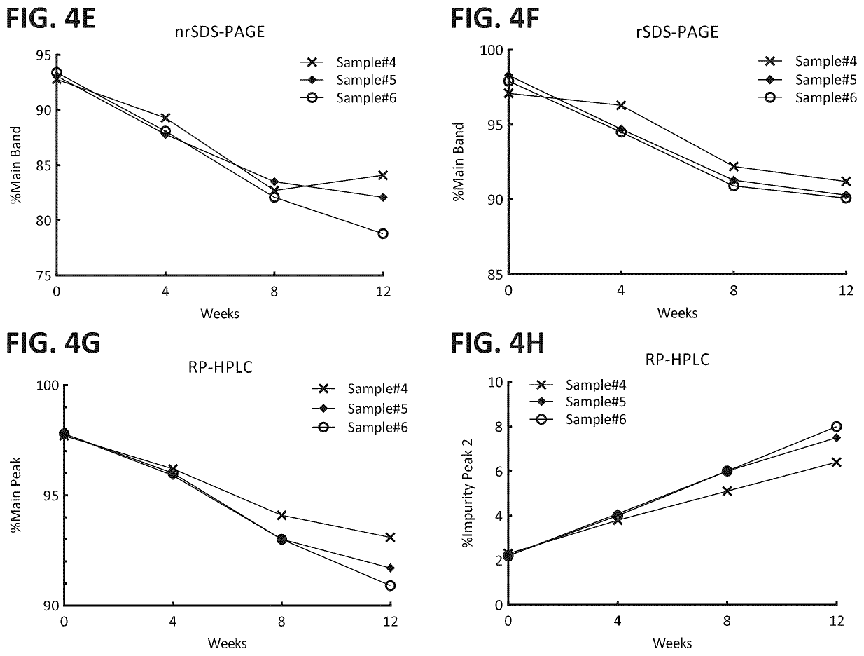

A decrease of 15.9 in percent main band of G-CSF-Fc dimer was observed by nrSDS-PAGE in Sample No. 3, which was higher in comparison to that of Sample No. 1 (6.3, FIG. 4A). rSDS-PAGE demonstrated a decrease of 10.4 in percent main band of G-CSF-Fc dimer in Sample No. 3, which was higher in comparison to that of Sample No. 1 (4.8, FIG. 4B). The percent main peak of G-CSF-Fc dimer by RP-HPLC decreased 9.1 in Sample No. 3, which was faster in comparison to Sample No. 1 (4.8, FIG. 4C). The main peak decreased with primarily increase of impurity peak2 observed by RP-HPLC (FIG. 4D). There was no significant difference between Sample No. 1 with 0.5 mM EDTA and Sample No. 2 with 1 mM EDTA, in presence of 10 mM acetate, 5% (w/v) sorbitol, 0.01% (w/v) PS 20, pH 5.2. These results indicate Sample Nos. 1 and 2 (which comprising 0.01% PS20) have better stability in comparison to Sample No. 3 (which comprising 0.03% PS20). The results suggest a formulation containing 10 mM sodium acetate, 5% (w/v) sorbitol, 0.01% (w/v) PS 20, either 0.5 mM or 1 mM EDTA, pH 5.2 is suitable for storage of G-CSF-Fc dimer.

The decrease in percent main band of G-CSF-Fc dimer by SDS-PAGE in Sample No. 6 (containing 0.03% PS20) was higher in comparison to that of Sample No. 4 (containing 0.01% PS20; FIGS. 4E-4F). The percent main peak of G-CSF-Fc dimer by RP-HPLC in Sample No. 6 decreased faster in comparison to that of Sample No. 4 (FIG. 4G). The main peak decreased with primarily increase of impurity peak2 observed by RP-HPLC (FIG. 4H). The results suggest a formulation containing 10 mM sodium acetate, 9% (w/v) sucrose, 0.01% (w/v) PS 20, 0.5 mM EDTA, pH 5.2 is suitable for storage of G-CSF-Fc dimers.

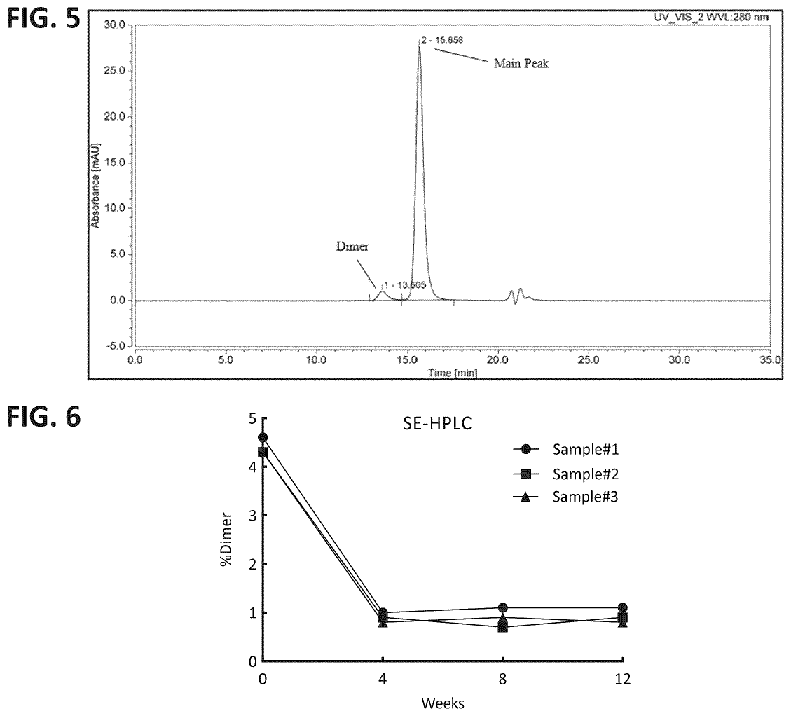

The results of SE-HPLC showed the overall change of the 6 samples over 12 weeks were less than 1%. An exemplary chromatogram of G-CSF-Fc dimer (Sample No. 2) at week 0 by SE-HPLC is shown in FIG. 5. As shown in FIG. 6, the data indicated that at elevated temperature (25±2° C.), there was a conversion from non-covalent dimer (formed by non-covalent bonding of two G-CSF-Fc dimers) to monomer as revealed by the increase of % main peak over time and concomitant reduction of % dimer contents in all samples.

In summary, the results show that the G-CSF-Fc dimer is most significantly degraded in Sample Nos. 3 and 6 (which both containing 0.03% PS20). Unexpectedly, high concentrations of PS20 (such as 0.03%) causes accelerated degradation of G-CSF-Fc dimer, while G-CSF-Fc dimer is more stable at 0.01% (w/v) PS20.

TABLE 10
Stability of G-CSF-Fc dimer in different formulation buffers

| | nr-SDS-PAGE | | | | r-SDS-PAGE | | | | SE-HPLC | | | | RP-HPLC | | | |
|---|---|---|---|---|---|---|---|---|---|---|---|---|---|---|---|---|
| Sample | % Main Band | | | | % Main Band | | | | % Main Peak | | | | % Main Peak | | | |
| No. | 0 w | 4 w | 8 w | 12 w | 0 w | 4 w | 8 w | 12 w | 0 w | 4 w | 8 w | 12 w | 0 w | 4 w | 8 w | 12 w |
| 1 | 91.8 | 89.6 | 80.3 | 85.5 | 96.4 | 96.1 | 91.3 | 91.6 | 95.4 | 99.0 | 98.9 | 98.9 | 97.9 | 96.3 | 93.9 | 92.5 |
| 2 | 93.1 | 90.4 | 81.8 | 85.8 | 96.8 | 95.2 | 91.1 | 90.9 | 95.7 | 99.1 | 99.3 | 99.1 | 97.8 | 96.1 | 93.1 | 91.9 |
| 3 | 92.3 | 86.6 | 79.5 | 76.4 | 97.4 | 95.1 | 89.4 | 87.0 | 95.7 | 99.2 | 99.1 | 99.2 | 98.0 | 95.6 | 91.8 | 88.9 |
| 4 | 92.8 | 89.3 | 82.7 | 84.1 | 97.1 | 96.3 | 92.2 | 91.2 | 95.5 | 98.8 | 98.9 | 98.9 | 97.7 | 96.2 | 94.1 | 93.1 |
| 5 | 93.1 | 87.8 | 83.5 | 82.1 | 98.3 | 94.7 | 91.3 | 90.3 | 95.7 | 99.1 | 99.1 | 99.1 | 97.8 | 95.9 | 93.0 | 91.7 |
| 6 | 93.4 | 88.1 | 82.1 | 78.8 | 97.9 | 94.5 | 90.9 | 90.1 | 95.7 | 98.9 | 98.9 | 99.1 | 97.8 | 96.0 | 93.0 | 90.9 |

### Example 6. Freeze-Thaw Stability Study

A freeze-thaw stability study was performed on two exemplary G-SCF-Fc dimer formulations. G-CSF-Fc dimer efbemalenograstim alfa with a concentration of 20 mg/mL was formulated in 10 mM sodium acetate, 0.01% (w/v) PS20, 1 mM EDTA, pH 5.2, containing 5% (w/v) sorbitol (Sample No. 1) or 9% (w/v) sucrose (Sample No. 2). The two formulation samples were filtered by using 0.2 μm syringe filter, and filled into staked needle pre-filled syringes. The syringes were frozen and thawed for a total of six times to room temperature. The samples at week 0 and after six freeze-thaw cycles were analyzed by nr-SDS-PAGE, r-SDS-PAGE, SE-HPLC and RP-HPLC.

TABLE 11
Freeze-thaw stability testing of G-CSF dimer pharmaceutical compositions.

| | nr-SDS-PAGE | | r-SDS-PAGE | | SE-HPLC | | RP-HPLC | |
|---|---|---|---|---|---|---|---|---|
| | % Main Band | | % Main Band | | % Main Peak | | % Main Peak | |
| Sample No. | 0 w | FT | 0 w | FT | 0 w | FT | 0 w | FT |
| 1 | 93.1 | 94.0 | 96.8 | 98.6 | 95.7 | 95.4 | 97.8 | 97.7 |
| 2 | 93.1 | 92.7 | 98.3 | 97.6 | 95.7 | 95.6 | 97.8 | 97.7 |

Sample#1: 20 mg/mL G-CSF-Fc dimer, 10 mM sodium acetate, 5% (w/v) sorbitol, 0.01% (w/v) PS 20, 1 mM EDTA, pH 5.2
Sample#2: 20 mg/mL G-CSF-Fc dimer, 10 mM sodium acetate, 9% (w/v) sucrose, 0.01% (w/v) PS 20, 1 mM EDTA, pH 5.2

As shown in Table 11, there were no significant changes observed for G-CSF-Fc dimer, after six cycles of freezing at −20° C. and thawing at room temperature. Thus, the G-CSF-Fc dimer pharmaceutical composition containing 20 mg/mL G-CSF-Fc dimer in 10 mM sodium acetate, either 5% (w/v) sorbitol or 9% (w/v) sucrose, 0.01% (w/v) PS20, 1 mM EDTA, pH 5.2 is sufficiently tolerant to repeated freeze-thaw cycles during the manufacturing process.

Based on the results of these studies, the formulation for the G-CSF-Fc dimer pharmaceutical composition was set as follows: 20 mg/mL G-CSF-Fc dimer in 10 mM sodium acetate, 1 mM EDTA, 5% (w/v) sorbitol, 0.01% (w/v) PS20, pH 5.2. Thus, the single-use pharmaceutical formulation designed as a solution for subcutaneous injection (S.C.) of G-CSF-Fc dimer was developed. Table 12 contains a list of excipients used in the pharmaceutical composition with corresponding concentrations and functions.

TABLE 12
Function of components of G-CSF-Fc
dimer pharmaceutical composition

| Ingredients | Target Concentration | | Function |
|---|---|---|---|
| G-CSF-Fc dimer | 20 | mg/mL | Active ingredient |
| Sodium acetate | 10 | mM | Buffering agent |
| PS20 | 0.01% | (w/v) | Surfactant |
| Sorbitol | 5% | (w/v) | Tonicity agent |
| EDTA | 1 | mM | Stabilizing agent |

| | | |
|---|---|---|
| Water for Injection | N/A | Solvent |

### Example 7. Batch Production of G-CDF-Fc Dimer in Pre-Filled Syringe

Liquid formulation of G-CSF-Fc dimer is produced as a sterile, single use, preservative free solution for convenient subcutaneous injection to deliver a target 1 mL (20 mg efbemalenograstim alfa) per pre-filled syringe. In additional of 20 mg/mL G-CSF-Fc dimer efbemalenograstim alfa, the solution also contains 10 mM sodium acetate, 1 mM EDTA, 5% (w/v) sorbitol, 0.01% (w/v) PS20 with a pH of 5.2. Each syringe contains 20 mg efbemalenograstim alfa in a sterile, clear, colorless, preservative-free solution (pH 5.2) containing acetate (0.6 mg), EDTA (0.29 mg), polysorbate 20 (0.1 mg), sodium (0.23 mg), and sorbitol (50 mg) in water for injection.

The storage temperature for the G-CSF-Fc dimer solution is −70° C.±10° C. The G-CSF-Fc dimer pharmaceutical composition has the same formulation and concentration as the G-CSF-Fc dimer solution, no formulation or dilution step is performed during the pharmaceutical manufacturing process. The batch size range is 5 kg to 15 kg, resulting in approximately 5,000 to 15,000 PFS of G-CSF-Fc dimer prefilled syringes. The batch formulation information for the G-CSF-Fc dimer composition is provided in Table 13.

TABLE 13
Batch formula for the G-CSF-Fc dimer pharmaceutical
composition in pre-filled syringes

| | Nominal amount per batch (g) | | | Nominal |
|---|---|---|---|---|
| | 5,000 | 10,000 | 15,000 | amount per |
| Ingredients | syringes | syringes | syringes | syringe (mg) |
| G-CSF-Fc | 100.0 | 200.0 | 300.0 | 20 |
| dimer | | | | |
| Sodium acetate | 3.25 | 6.50 | 9.75 | 0.65 |
| Glacial | 0.65 | 1.30 | 1.95 | 0.13 |
| acetic acid | | | | |
| PS20 | 0.50 | 1.00 | 1.50 | 0.1 |
| Sorbitol | 250.00 | 500.00 | 750.00 | 50 |
| EDTA-2Na | 1.69 | 3.38 | 5.07 | 0.338 |
| H₂O | q.s. to 5 kg | q.s. to 10 kg | q.s. to 15 kg | q.s. to 1 mL |

q.s.: quantum satis (as much as may suffice)

G-CSF-Fc dimer solution at a concentration of 20 mg/mL was thawed and mixed, and was filtered using two sets of 0.45/0.2 μm filters (Sartopore Midicap) connected in series in an ISO 5 (grade A) environment. The dimer solution was sterile filtered into sterile bags (Flexboy®). After filtration, an automatic syringe filler (INOVA H3-5) was used to fill sterile liquid into a 1 mL glass needle syringe (BD MEDICAL PHARMACEUTICAL SYSTEMS, CAT #47460019) to approximately 1.05 mL. Syringe stoppers were installed, and the syringes were stored at 2-8° C.

### Example 8. Long Term Stability of the G-CSF-Fc Dimer Pharmaceutical Composition

The G-CSF-Fc dimer pharmaceutical composition is a liquid formulation supplied in 1 mL glass syringe (for example, BD MEDICAL PHARMACEUTICAL SYSTEMS, CAT #47460019) containing approximately 20 mg/mL G-CSF-Fc dimer efbemalenograstim alfa formulated in 10 mM sodium acetate, 1 mM EDTA, 5% (w/v) sorbitol, 0.01% (w/v) PS20, pH 5.2.

The pharmaceutical composition was evaluated for long term storage at 5±3° C. for up to 36 months. Additionally, accelerated stability study was performed at 25±2° C. (60±5% relative humidity) for up to 6 months. SE-HPLC, rCE-SDS, nrCE-SDS and icIEF were used to analyze the degradation and charge heterogeneity of the G-CSF-Fc dimer. The G-CSF-Fc dimer pharmaceutical composition has an osmolality of about 311 mOsm/kg. The osmolality was determined using the method descried in the United States Pharmacopeia <785>.

### Procedures and Acceptance Criteria

All samples were analyzed using the test procedures listed in Table 14.

TABLE 14
Test procedures and acceptance Criteria

| Procedure | Acceptance Criteria |
|---|---|
| Appearance | Clear, colorless to slightly |
| | yellowish solution, essentially |
| | free of visible particulates. |
| pH | 5.0-5.4 |
| Particulate Matter | ≥10 μm: ≤6000 per vial |
| (particles/container) | ≥25 μm: ≤600 per vial |
| Purity by SE-HPLC (%) | Main Peak ^{a}: ≥ 96 |
| | Dimer ^{b}: ≤2.5 |
| | Aggregate: ≤2.0 |
| Purity by rCE-SDS (%) | Main peak: ≥95.5 |
| | LMWS: ≤4.0 |
| | HMWS: ≤1.5 |
| Purity by nrCE-SDS (%) | Main Peak: ≥94.0 |
| | LMWS: ≤6.0 |
| | HMWS: ≤1.5 |
| Purity by icIEF (%) | Acidic peaks: 27.2-39.0 |
| | Main peak: 36.5-60.1 |
| | Basic peaks: 7.2-30 |
| Potency by Proliferation | 70-130 |
| Assay (% relative potency) | |
| Protein Concentration by | 20.0 ± 2.0 (mg/mL) |
| UV Spec Scan | |

^{a} G-CSF-Fc dimer
^{b} Dimer is formed by non-covalent bonding of two G-CSF-Fc dimers.

### Appearance

Samples were placed into colorimetric cuvettes and examined by eye against a black and a white background, respectively, for visible particles. Samples were compared to colorimetric standards against a white background to evaluate their colors.

pH

The pH values of the samples were determined using a pH meter (ESCO Class II) calibrated with standards at pH 6.86 and pH 4.01.

### Particle Size

Particles in the samples were examined using particle sizing systems (ACCUSIZER 780 SIS).

rCE-SDS (Reduced Capillary Electrophoresis Sodium Dodecyl Sulfate)

The rCE-SDS was performed as follows. 1 M β-mercaptoethanol and 1% SDS sample buffer (Beckman Coulter, Cat #A10663) were mixed to make a reducing buffer. Samples were diluted to about 4 mg/mL and mixed with the reducing buffer at a volume ratio of 1:3 to a final concentration of about 1 mg/mL followed by incubation at 65±3° C. for 5±1 minutes and a subsequent ice bath for 15±2 minutes. Water was used as blank control.

Uncoated capillary with an inner diameter of 50 μm and a length of 30.2 cm (effective length 20.2 cm). Before electrophoresis, the capillary column was filled with hydrogel and washed with 0.1 mol/L sodium hydroxide, 0.1 mol/L hydrochloric acid, and ultrapure water at 70 psi. Samples were injected −5 kV for 20 seconds and ran at a separation voltage of −15 kV for 35 minutes. The capillary column temperature was 20° C. and the detection wavelength was 220 nm.

nrCE-SDS (Non-Reduced SDS Electrophoresis)

The nrCE-SDS was performed as follows. A non-reducing premix solution was prepared by mixing 100 mM Tris-HCl solution (pH 9.0) and 1% SDS sample buffer (Beckman Coulter, Cat #A10663). Samples were diluted to approximately 4 mg/mL and mixed with a non-reducing premix solution at a ratio of 1:3 to a final concentration of approximately 1 mg/mL followed by incubation at 65±3° C. for 5±1 minutes and a subsequent ice bath for 15±2 minutes. Water was used as blank control.

Uncoated capillary with an inner diameter of 50 μm and a length of 30.2 cm (effective length 20.2 cm). Before electrophoresis, the capillary column was filled with hydrogel and washed with 0.1 mol/L sodium hydroxide, 0.1 mol/L hydrochloric acid, and ultrapure water at 70 psi, respectively.

Samples were injected −5 kV for 20 seconds and ran at a separation voltage of −15 kV for 35 minutes. The capillary column temperature was 20° C. and the detection wavelength was 220 nm.

### Imaged Capillary Isoelectric Focusing

Imaged capillary Isoelectric Focusing electrophoresis (icIEF) was performed as follows. The charge heterogeneity analysis of the G-CSF-Fc dimer was performed at 280 nm using an iCE3 imaging capillary isoelectric focusing electrophoresis instrument (ProteinSimple) with a 100 μm×5 cm fluorocarbon-coated capillary (ProteinSimple). The G-CSF dimer samples were diluted with ultrapure water to a protein concentration of 5.0 mg/mL. 25 μL of the diluted sample, 2.5 μL sialidase (Prozyme), 10 μL 5× reaction buffer (Prozyme), and 12.5 μL of ultrapure water were mixed well, and incubated at 37° C. for 5 hours for enzymatic digestion. Pretreatment for the next steps was then performed after enzymatic digestion by mixing 70.0 μL of 1% methylcellulose, 2.0 μL Pharmalyte3-10, 8.0 μL Pharmalyte5-8, 0.5 μL pI marker (4.65), 0.5 μL pI marker (7.65), 79.0 μL ultrapure water, and 40.0 μL sample into EP tube. The mixture was centrifuged at 12,000 rpm for 3 minutes. 160 μL of the supernatant was transferred into a sample bottle equipped with an inner cannula. The control was processed simultaneously.

The sample was focused by introducing a potential of 1500 V for 1 min, followed by a potential of 3000 V for 6 min. The anolyte was 80 mM phosphoric acid, and the catholyte was 100 mM sodium hydroxide, both in 0.1% methylcellulose. Images of the focused charge variants were obtained by passing 280 nm ultraviolet (UV) light through a capillary and into the lens of a charge-coupled device digital camera. The data were analyzed using Waters Empower3 software.

### Proliferation Assay

G-CSF-Fc dimer can activate the STAT3 signaling pathway in M-NFS-60 cells and stimulate cell proliferation. Therefore, Cell Titer Blue reagent (Promega Corporation, Cat #G8080) staining method was used to detect the effect of G-CSF-Fc dimer on proliferation of M-NFS-60 cells (ATCC. CRL-1838). Proliferation assay of M-NFS-60 cells by G-CSF-Fc dimer: M-NFS-60 cells were washed using RPMI 1640 medium, and resuspended in RPMI 1640 medium containing 10% FBS, 2 mM L-glutamine, 1% penicillin/streptomycin, and incubated in shake flasks at 37° C., 5% CO₂ for 1-2 hours. The cell suspension was diluted to 7×10⁵ cells/mL, seeded in 96-well plates containing serially diluted standards and G-CSF-Fc dimer samples, and incubated at 37° C., 5% CO₂ for about 48 hours. 20 μL of Cell Titer Blue reagent (Promega Corporation, Cat #G8080) was then added to the 96-well plate and incubated for 4 hours at 37° C., 5% CO₂. The 96-well plates were detected at 570/600 nm using a microplate reader (Molecular device, Versa Max). The optimal density (OD) units were plotted against the G-CSF-Fc dimer concentrations. The curves were fitted using a 4-parameter logistic model and the EC50 values were obtained. The bioassay results were expressed as a percentage of the EC50 of the reference standard sample divided by the EC50 of the stability time point sampled sample.

### Protein Concentration (Strength)

Protein concentration or strength was measured using a visible spectrophotometer (GE, model: Gene quant-100) by absorption. The G-CSF dimer samples were diluted with buffer to a concentration of about 0.5 mg/mL, and ultrapure water was used as a blank control. 400 μL of the sample were transferred to a cuvette, and measured at 320 nm and 280 nm for their absorbance values. The protein concentration was calculated using an extinction coefficient (F) of 1.084 (mg/mL)⁻¹ cm⁻¹. Protein concentration=(A280−A320)/molar extinction coefficient×dilution factor.

Results of the experiments above are shown in Table 15 and Table 16.

TABLE 15
Stability of G-CSF-Fc dimer pharmaceutical composition in PFS stored at 5 ± 3° C.

| | | Time Interval (months) | | | | | | | | |
|---|---|---|---|---|---|---|---|---|---|---|
| Items | | 0 | 3 | 6 | 9 | 12 | 18 | 24 | 30 | 36 |
| Appearance | | pass | pass | pass | pass | pass | pass | pass | pass | pass |
| pH | | 5.1 | 5.2 | 5.1 | 5.1 | 5.1 | 5.2 | 5.3 | 5.2 | 5.2 |
| Particulate Matter | ≥10 μm: | 296 | NP | 329 | NP | 129 | 533 | 346 | 271 | 524 |
| (particles/container) | ≥25 μm | 11 | NP | 5 | NP | 5 | 16 | 7 | 10 | 17 |
| SE-HPLC (%) | Main Peak | 98.2 | 98.1 | 98.2 | 98.1 | 97.9 | 97.9 | 98.2 | 98.0 | 97.7 |
| | Dimer | 1.2 | 1.3 | 1.3 | 1.4 | 1.4 | 1.4 | 1.5 | 1.6 | 1.6 |
| | Aggregate | 0.6 | 0.6 | 0.5 | 0.4 | 0.6 | 0.5 | 0.2 | 0.2 | 0.4 |
| rCE-SDS (%) | Main peak | 98.5 | 96.9 | 97.7 | 97.3 | 97.6 | 97.9 | 97.1 | 97.3 | 97.1 |
| | LMWS | 1.1 | 2.6 | 1.8 | 2.3 | 2.0 | 1.5 | 2.6 | 2.4 | 2.7 |
| | HMWS | 0.4 | 0.5 | 0.5 | 0.4 | 0.3 | 0.6 | 0.3 | 0.4 | 0.2 |
| nrCE-SDS (%) | Main Peak | 97.5 | 96.6 | 96.7 | 96.3 | 96.6 | 96.1 | 95.6 | 95.5 | 95.2 |
| | LMWS | 2.4 | 3.2 | 3.1 | 3.5 | 3.3 | 3.6 | 4.1 | 4.2 | 4.6 |
| | HMWS | 0.1 | 0.2 | 0.2 | 0.2 | 0.2 | 0.2 | 0.3 | 0.3 | 0.3 |
| icIEF (%) | Acidic peaks | 34.4 | 34.7 | 32.3 | 33.5 | 33.5 | 33.0 | 32.3 | 37.0 | 35.0 |
| | Main peak | 51.6 | 51.1 | 51.7 | 52.0 | 52.8 | 51.1 | 54.8 | 47.7 | 50.3 |
| | Basic peaks | 13.9 | 14.2 | 15.9 | 14.5 | 13.7 | 15.9 | 12.9 | 15.4 | 14.7 |

| | | | | | | | | | |
|---|---|---|---|---|---|---|---|---|---|
| Potency (%) | 88 | 116 | 105 | 110 | 110 | 100 | 114 | 90 | 102 |
| Concentration (mg/mL) | 20.0 | 19.9 | 20.0 | 20.0 | 20.1 | 19.8 | 19.9 | 19.8 | 20.0 |

TABLE 16
Stability of the G-CSF-Fc dimer pharmaceutical composition
in PFS stored at 25 ± 2° C.

| | | Time Interval (months) | | | | |
|---|---|---|---|---|---|---|
| Items | | 0 | 1 | 2 | 3 | 6 |
| Appearance | | pass | pass | pass | pass | pass |
| pH | | 5.1 | 5.2 | 5.1 | 5.3 | 5.1 |
| Particulate Matter | ≥10 μm | 296 | NP | NP | NP | 307 |
| (particles/container) | ≥25 μm | 11 | NP | NP | NP | 17 |
| SE-HPLC | Main Peak | 99.4 | 99.6 | 99.5 | 98.4 | 99.0 |
| (%) | Dimer | 1.2 | 1.4 | 1.5 | 1.7 | 1.6 |
| | Aggregate | 0.6 | 0.4 | 0.4 | 1.4 | 0.6 |
| rCE-SDS | Main peak | 98.5 | 97.7 | 96.3 | 96.4 | 94.1 |
| (%) | LMWS | 1.1 | 1.9 | 2.9 | 3.0 | 5.2 |
| | HMWS | 0.4 | 0.4 | 0.8 | 0.6 | 0.7 |
| nrCE-SDS | Main peak | 97.5 | 96.1 | 95.4 | 93.5 | 90.5 |
| (%) | LMWS | 2.4 | 3.3 | 4.5 | 5.9 | 8.5 |
| | HMWS | 0.1 | 0.6 | 0.1 | 0.6 | 1.0 |
| icIEF | Acidic peaks | 34.4 | 34.1 | 32.7 | 35.3 | 34.7 |
| (%) | Main peak | 51.6 | 51.2 | 52.4 | 50.5 | 50.2 |
| | Basic peaks | 13.9 | 14.7 | 14.9 | 14.2 | 15.1 |

| | | | | | |
|---|---|---|---|---|---|
| Potency (%) | 88 | 93 | 93 | 116 | 100 |
| Concentration (mg/mL) | 20.0 | 20.0 | 19.5 | 19.9 | 20.0 |

NP: Not Performed

The results of all testing items for the G-CSF-Fc dimer pharmaceutical composition were within the acceptance criteria after stored at 5±3° C. for up to 36 months. The stability data supported a shelf life of 36 months at 5±3° C. for the G-CSF-Fc dimer pharmaceutical composition. No significant changes were observed in the G-CSF-Fc dimer pharmaceutical composition for up to 3 months of storage at 25±2° C. When stored at 25±2° C. for up to 6 months, a 4.4% decrease in the main peak was observed by rCE-SDS with primarily a 4.1% increase in lower molecular weight species (LMWS). A 7.0% decrease in the main peak was observed by nrCE-SDS, with primarily a 6.1% increase in low molecular weight species (LMWS).

Based on the results of stability studies, the G-CSF-Fc dimer pharmaceutical composition is stable over 36 months at 5° C.±3° C. The initial shelf life for the G-CSF-Fc dimer pharmaceutical composition is currently set at 36 months if stored at the recommended storage temperature of 2° C. to 8° C., protected from light. The shelf life may be extended based on available stability data.

The above are examples of methods and compositions of the invention. It is understood that various other embodiments may be practiced, given the general description provided above, and the examples are not intended to limit the scope of the claims.

SEQUENCE LISTING

| SEQ | | |
|---|---|---|
| ID | | |
| NO: | Description | Sequence |
| 1. | G-CSF | TPLGPASSLPQSFLLKCLEQVRKIQGDGAALQEKLCATYKLCHPEELVLLGHSLGIPWA |
| | monomer | PLSSCPSQALQLAGCLSQLHSGLFLYQGLLQALEGISPELGPTLDTLQLDVADFATTIW |
| | | QQMEELGMAPALQPTQGAMPAFASAFQRRAGGVLVASHLQSFLEVSYRVLRHLAQP |
| 2. | IgG2 Fc (P331S) | 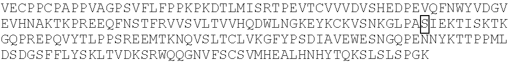 |
| 3. | Long IgG2 Fc (P331S) | 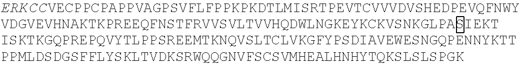 |
| 4. | IgG1 Fc (L234A, L235A, P331S) | 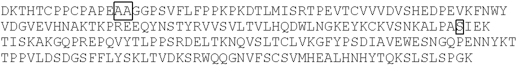 |
| 5. | IgG4 Fc (S228P, L234A, L235A) | 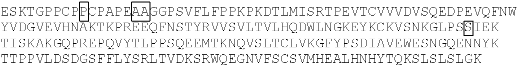 |
| 6. | G-CSF- linker- IgG2 Fc (P331S) (F-627; 413aa) | 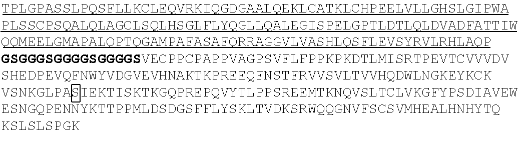 |
| 7. | G-CSF- linker-long IgG2 Fc (P331S) (418aa) | 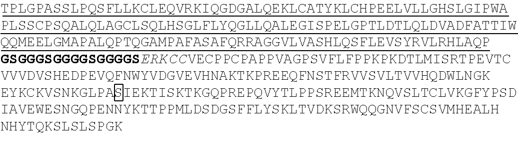 |
| 8. | G-CSF- linker- IgG1 fc (L234A, L235A, P331S) | 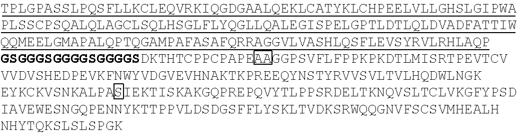 |
| 9. | G-CSF- linker IgG4 Fc (S228P, L234A, L235A) | 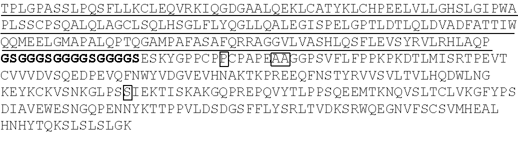 |
| 10. | G-CSF- linker- IgG2 Fc (P331S) (412aa) | 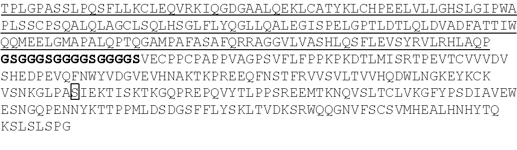 |
| 11. | DNA | ACCCCCCTGGGCCCTGCCAGCTCCCTGCCCCAGAGCTTCCTGCTCAAGTGCTTAGA |
| | sequence | GCAAGTGAGGAAGATCCAGGGCGATGGCGCAGCGCTCCAGGAGAAGCTGTGTGCC |
| | encoding | ACCTACAAGCTGTGCCACCCCGAGGAGCTGGTGCTGCTCGGACACTCTCTGGGCAT |
| | G-CSF | CCCCTGGGCTCCCCTGAGCAGCTGCCCCAGCCAGGCCCTGCAGCTGGCAGGCTGCT |
| | monomer | TGAGCCAACTCCATAGCGGCCTTTTCCTCTACCAGGGGCTCCTGCAGGCCCTGGAA |
| | with Fc | GGGATCTCCCCCGAGTTGGGTCCCACCTTGGACACACTGCAGCTGGACGTCGCCGA |
| | fragment | CTTTGCCACCACCATCTGGCAGCAGATGGAAGAACTGGGAATGGCCCCTGCCCTGC |
| | | AGCCCACCCAGGGTGCCATGCCGGCCTTCGCCTCTGCTTTCCAGCGCCGGGCAGGA |
| | | GGGGTCCTGGTTGCCTCCCATCTGCAGAGCTTCCTGGAGGTGTCGTACCGCGTTCTA |
| | | CGCCACCTTGCCCAGCCCGGATCCGGTGGCGGTTCCGGTGGAGGCGGAAGCGGCG |
| | | GTGGAGGATCAGTCGAGTGCCCACCGTGCCCAGCACCACCTGTGGCAGGACCGTCA |
| | | GTCTTCCTCTTCCCCCCAAAACCCAAGGACACCCTCATGATCTCCCGGACCCCTGAG |
| | | GTCACGTGCGTGGTGGTGGACGTGAGCCACGAAGACCCCGAGGTCCAGTTCAACT |
| | | GGTACGTGGACGGCGTGGAGGTGCATAATGCCAAGACAAAGCCACGGGAGGAGCA |
| | | GTTCAACAGCACGTTCCGTGTGGTCAGCGTCCTCACCGTTGTGCACCAGGACTGGC |
| | | TGAACGGCAAGGAGTACAAGTGCAAGGTCTCCAACAAAGGCCTCCCAGCCTCCATC |
| | | GAGAAAACCATCTCCAAAACCAAAGGGCAGCCCCGAGAACCACAGGTGTACACCC |
| | | TGCCCCCATCCCGGGAGGAGATGACCAAGAACCAGGTCAGCCTGACCTGCCTGGTC |
| | | AAAGGCTTCTACCCCAGCGACATCGCCGTGGAGTGGGAGAGCAATGGGCAGCCGG |
| | | AGAACAACTACAAGACCACACCTCCCATGCTGGACTCCGACGGCTCCTTCTTCCTCT |
| | | ACAGCAAGCTCACCGTGGACAAGAGCAGGTGGCAGCAGGGGAACGTCTTCTCATG |
| | | CTCCGTGATGCATGAGGCTCTGCACAACCACTACACGCAGAAGAGCCTCTCCCTGT |
| | | CTCCGGGTAAA |
| 12. | Linker | GSGGGSGGGGSGGGGS |
| 13. | Linker | ASTKGP |
| 14. | Linker | GGSGGS |
| 15. | Linker | SGGGGS |
| 16. | Linker | GRAGGGGAGGGG |
| 17. | Linker | GRAGGG |
| 18. | Linker | (G)ₙ, n is an integer of at least 1 |
| 19. | Linker | (GS)ₙ, n is an integer of at least 1 |
| 20. | Linker | (GSGGS)ₙ, n is an integer of at least 1 |
| 21. | Linker | GPGPGP |
| 22. | Linker | (GGGGS)ₙ, n is an integer of at least 1 |
| 23. | Linker | GG |
| 24. | Linker | GGSG |
| 25. | Linker | GGSGG |
| 26. | Linker | GSGSG |
| 27. | Linker | GSGGG |
| 28. | Linker | GGGSG |
| 29. | Linker | GSSSG |
| 30. | Linker | GGGGSGGGGSGGGGS |
| 31. | Linker | GGGGS |
| 32. | hinger | ERKCC |

1. A pharmaceutical composition comprising:
(a) about 1 mg/mL to about 100 mg/mL granulocyte colony-stimulating factor (G-CSF) dimer;
(b) about 1 mM to about 50 mM buffering agent;
(c) about 0.1 mM to about 20 mM EDTA;
(d) about 1% (w/v) to about 15% (w/v) sorbitol; and
(e) about 0.001% (w/v) to about 0.1% (w/v) surfactant, wherein the pH of the pharmaceutical composition is about 4.2 to about 6.2.

2. (canceled)

3. The pharmaceutical composition of claim 1, wherein the G-CSF dimer comprises two monomeric subunits, each comprising a G-CSF monomer and a dimerization domain, wherein the G-CSF monomer comprises the sequence of SEQ ID NO: 1.

4-6. (canceled)

7. The pharmaceutical composition of claim 3, wherein the dimerization domain comprises at least a portion of an Fc fragment.

8. (canceled)

9. The pharmaceutical composition of claim 7, wherein the Fc fragment comprises an amino acid sequence selected from the group consisting of SEQ ID NOs: 2-5.

10. (canceled)

11. The pharmaceutical composition of claim 3, wherein each monomeric subunit comprises an amino acid sequence of any of SEQ ID NOs: 6-10, or a variant thereof having at least about 90% sequence identity to the amino acid sequence of any of SEQ ID NOs: 6-10.

12. The pharmaceutical composition of claim 11, wherein each monomeric subunit comprises the amino acid sequence of SEQ ID NO: 6.

13. The pharmaceutical composition of claim 1, wherein the G-CSF dimer is efbemalenograstim alfa.

14. The pharmaceutical composition of claim 1, wherein the concentration of the G-CSF dimer is about 5 mg/mL to about 50 mg/mL.

15. The pharmaceutical composition of claim 1, wherein the concentration of the G-CSF dimer is about 20 mg/mL.

16. The pharmaceutical composition of claim 1, wherein the buffering agent is sodium acetate.

17. The pharmaceutical composition of claim 16, wherein the concentration of the sodium acetate is about 1 mM to about 30 mM.

18. The pharmaceutical composition of claim 16, wherein the concentration of the sodium acetate is about 10 mM.

19. (canceled)

20. The pharmaceutical composition of claim 1, wherein the concentration of the EDTA is about 0.1 mM to about 10 mM.

21. The pharmaceutical composition of claim 20, wherein the concentration of the EDTA is about 1 mM.

22. (canceled)

23. The pharmaceutical composition of claim 1, wherein said pharmaceutical composition comprises about 1% (w/v) to about 8% (w/v) sorbitol.

24. The pharmaceutical composition of claim 1, wherein said pharmaceutical composition comprises about 5% (w/v) sorbitol.

25-28. (canceled)

29. The pharmaceutical composition of claim 1, wherein the surfactant is polysorbate 20 (PS20).

30. The pharmaceutical composition of claim 29, wherein said pharmaceutical composition comprises about 0.005% (w/v) to about 0.05% (w/v) PS20.

31. The pharmaceutical composition of claim 29, wherein said pharmaceutical composition comprises about 0.01% (w/v) PS20.

32-34. (canceled)

35. The pharmaceutical composition of claim 1, wherein the pH of the pharmaceutical composition is about 5.2.

36. The pharmaceutical composition of claim 13, wherein the pharmaceutical composition comprises:
(a) about 20 mg/mL of the G-CSF dimer;
(b) about 10 mM sodium acetate;
(c) about 1 mM EDTA;
(d) about 5% (w/v) sorbitol; and
(e) about 0.01% (w/v) PS20;
wherein the pharmaceutical composition has a pH of about 5.2.

37-40. (canceled)

41. A method of treating or preventing a disease or condition in an individual, comprising administering to the individual an effective amount of the pharmaceutical composition of claim 1.

42-51. (canceled)

52. A method of manufacturing the pharmaceutical composition of claim 1, comprising:
(i) culturing a host cell under a condition suitable for expressing the G-CSF dimer;
(ii) isolating the expressed G-CSF dimer from the cell culture;
(iii) purifying the expressed G-CSF dimer; and
(iv) formulating the purified G-CSF dimer with the buffering agent, the stabilizing agent, the tonicity agent, and the surfactant.

53. (canceled)
